(19) United States
(12) Patent Application Publication
(10) Pub. No.: US 2026/0215797 A1
(43) Pub. Date: Jul. 30, 2026

(54) HANDLES FOR MEDICAL DEVICES, MEDICAL DEVICES, METHODS FOR OPERATING MEDICAL DEVICES

(71) Applicants: HANGZHOU AGS MEDTECH CO., LTD., Hangzhou (CN); HAINAN JUNLEMING PHARMACEUTICAL TECHNOLOGY DEVELOPMENT CO., LTD., Sanya (CN)

(72) Inventors: Feng YAO, Hangzhou (CN); Jiawei WU, Hangzhou (CN); Jiaqi LI, Hangzhou (CN); Guoqing WANG, Hangzhou (CN)

(73) Assignees: HANGZHOU AGS MEDTECH CO., LTD., Hangzhou (CN); HAINAN JUNLEMING PHARMACEUTICAL TECHNOLOGY DEVELOPMENT CO., LTD., Sanya (CN)

(21) Appl. No.: 19/574,254

(22) Filed: Mar. 20, 2026

Related U.S. Application Data
(63) Continuation-in-part of application No. PCT/CN2024/120234, filed on Sep. 21, 2024.

(30) Foreign Application Priority Data
Sep. 22, 2023 (CN) 202311236687.4

(51) Int. Cl. A61B 17/29; A61B 17/00

(52) U.S. Cl. CPC A61B 17/2909; A61B 2017/00367; A61B 2017/00477; A61B 2017/2903; A61B 2017/2912

(57) ABSTRACT

A handle for a medical device is provided. The handle includes a handheld portion, a driving portion, and a plurality of transmission portions configured to be operatively coupled to a plurality of moving components at a distal end of the medical device. The handheld portion is slidably engaged with the driving portion, and the driving portion is switchably connected to at least one of the plurality of transmission portions.

### CROSS-REFERENCE TO RELATED APPLICATIONS

This application is a Continuation-in-part of International Application No. PCT/CN2024/120234, filed on Sep. 21, 2024, which claims priority to Chinese Patent Application No. 202311236687.4, filed on Sep. 22, 2023, the entire contents of each of which are hereby incorporated by reference.

### TECHNICAL FIELD

The present disclosure generally relates to the field of medical devices, and in particular to a handle for a medical device, a medical device, and a method for operating a medical device.

### BACKGROUND

Medical devices for endoscopic surgery mostly include an independent moving component, and the independent moving component can only be controlled by a corresponding independent operation component. That is, when a handle for a medical device includes an operation component, the operation component can control only one moving component at the distal end. To control a plurality of moving components at the distal end, an operation component needs to be added to one handle, or a plurality of handles are directly used.

However, when a plurality of operation components are used to operate the plurality of moving components at the distal end, an operator needs to perform an operation with a plurality of hands, which brings inconvenience to clinical application. The plurality of operation components not only increase complexity and difficulty of the operation, but also cause coordination difficulties and operation errors, affecting the accuracy and safety of the operation.

Therefore, it is desirable to provide a handle that facilitates control of a plurality of moving components and a medical device including the handle.

### SUMMARY

One or more embodiments of the present disclosure provide a handle for a medical device. The handle includes a handheld portion, a driving portion, and a plurality of transmission portions configured to be operatively coupled to a plurality of moving components at a distal end of the medical device. The handheld portion is slidably engaged with the driving portion, and the driving portion is switchably connected to at least one of the plurality of transmission portions, and the driving portion is switchable between being connected to only one of the plurality of transmission portions and being simultaneously connected to at least two of the plurality of transmission portions.

One or more embodiments of the present disclosure provide a medical device. The medical device includes a handle for a medical device according to any embodiment of the present disclosure, a delivery assembly, and a plurality of moving components disposed at a distal end of the delivery assembly. A proximal end of the delivery assembly is connected to the handle. Each of the plurality of moving components is drivingly connected to at least one of the plurality of transmission portions.

One or more embodiments of the present disclosure provide a method for operating a medical device. The method is applied to the medical device according to any embodiment of the present application. The method includes controlling a driving portion and a plurality of transmission portions of a handle to be located at a preset position; driving the plurality of transmission portions to rotate about a first direction until a target transmission portion of the plurality of transmission portions engages with the driving portion; and controlling the driving portion to drive the target transmission portion to slide along the first direction to control at least one of the plurality of moving components connected to the target transmission portion to perform an operation.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and where:

FIG. 1 is a schematic diagram illustrating a structure of a handle according to some embodiments of the present disclosure;

FIG. 2 is an exploded view of a handle according to some embodiments of the present disclosure;

FIG. 3 is a schematic diagram of a cross-section taken along line A-A' in FIG. 1;

FIG. 4A is a cross-sectional view of a connection between a driving portion and a transmission portion according to some embodiments of the present disclosure;

FIG. 4B is an enlarged view of a portion B in FIG. 4A;

FIG. 5 is a schematic diagram illustrating a structure of a handle according to some other embodiments of the present disclosure;

FIG. 6 is an exploded view of a handle according to some embodiments of the present disclosure;

FIG. 7 is a schematic diagram illustrating a structure of a handheld portion according to some embodiments of the present disclosure;

FIG. 8A is a schematic diagram illustrating a structure of a rotary cap according to some embodiments of the present disclosure;

FIG. 8B is a cross-sectional view of FIG. 8A;

FIG. 9 is schematic diagram of a cross-section taken along line D-D' in FIG. 5;

FIG. 10 is an enlarged view of a portion E shown in FIG. 9;

FIG. 11 is an enlarged view of a portion E' shown in FIG. 9;

FIG. 12 is a schematic diagram illustrating a structure of a handle according to still some other embodiments of the present disclosure;

FIG. 13 is an exploded view of a handle according to some embodiments of the present disclosure;

FIG. 14 is a schematic diagram of a connection between an inner ring sleeve with a first positioning assembly and a second positioning assembly according to some embodiments of the present disclosure;

FIG. 15 is a schematic diagram illustrating a structure of a handheld portion according to still some other embodiments of the present disclosure;

FIG. 16 is a schematic diagram of a cross-section taken along line G-G' in FIG. 15;

FIG. 17A is an enlarged view of a portion G1 in FIG. 16;

FIG. 17B is a schematic diagram of a first positioning assembly and a second positioning assembly according to still some other embodiments of the present disclosure;

FIG. 18 is a schematic diagram illustrating a structure of a handheld portion and a driving portion according to still some other embodiments of the present disclosure;

FIG. 19 is a schematic diagram of a cross-section taken along line B-B of a handheld portion and a driving portion when the driving portion is located at a preset position, according to some embodiments of the present disclosure;

FIG. 20 is an enlarged view of a portion C in FIG. 19;

FIG. 21 is a schematic diagram of a cross-section taken along line B-B of a handheld portion and a driving portion when the driving portion drives the transmission portion into a release space, according to some embodiments of the present disclosure;

FIG. 22 is an enlarged view of a portion D in FIG. 21;

FIG. 23 is a schematic diagram illustrating a structure of a handle according to still some other embodiments of the present disclosure;

FIG. 24 is a schematic diagram of a cross-section taken along line A-A of the handle shown in FIG. 23;

FIG. 25 is a schematic diagram of a connection between a rotary cap and a handheld portion according to some embodiments of the present disclosure;

FIG. 26 is an enlarged view of a portion H in FIG. 25;

FIG. 27 is a schematic diagram illustrating a structure of a locking groove according to some embodiments of the present disclosure;

FIG. 28 is a schematic diagram illustrating a structure of a locking block according to some embodiments of the present disclosure;

FIG. 29 is an exploded view of a handle according to some embodiments of the present disclosure;

FIG. 30 is a cross-sectional view of a support rod provided with a blocking portion according to some embodiments of the present disclosure;

FIG. 31 is a schematic diagram illustrating a structure of a support rod according to some embodiments of the present disclosure;

FIG. 32 is a schematic diagram illustrating a structure of a limiting portion abutting against a blocking portion according to some embodiments of the present disclosure;

FIG. 33 is a schematic diagram illustrating a structure of a blocking portion located at a release position according to some embodiments of the present disclosure;

FIG. 34 is a schematic diagram illustrating a structure of a handheld portion according to some embodiments of the present disclosure;

FIGS. 35A-35D are schematic diagrams each illustrating a structure of a handle in a first gear according to some embodiments of the present disclosure;

FIGS. 36A-36D are schematic diagrams each illustrating a structure of a handle in a second gear according to some embodiments of the present disclosure;

FIGS. 37A-37D are schematic diagrams each illustrating a structure of a handle in a third gear according to some embodiments of the present disclosure;

FIG. 38 is an exploded view of a handle according to some embodiments of the present disclosure;

FIG. 39 is a schematic diagram of a locking member engaged with a handheld portion according to some embodiments of the present disclosure;

FIG. 40 is a cross-sectional view of a locking member engaged with a handheld portion according to some embodiments of the present disclosure;

FIG. 41 is a schematic diagram of a locking member separated from a handheld portion according to some embodiments of the present disclosure;

FIG. 42A is a schematic diagram of a locking member in a locked state according to some embodiments of the present disclosure;

FIG. 42B is a schematic diagram of a locking member in an unlocked state according to some embodiments of the present disclosure;

FIG. 43 is a schematic diagram illustrating a structure of a medical device according to some embodiments of the present disclosure;

FIG. 44 is a schematic diagram illustrating a structure of a delivery assembly according to some embodiments of the present disclosure;

FIG. 45 is a schematic diagram illustrating a structure of a movement change of the delivery assembly in FIG. 44;

FIG. 46 is a flowchart illustrating an exemplary process of a method for operating a medical device according to some embodiments of the present disclosure;

FIG. 47 is a schematic diagram illustrating a structure of a medical device according to some embodiments of the present disclosure;

FIG. 48 is a schematic diagram illustrating a structure of a medical device during an operation process according to some embodiments of the present disclosure;

FIG. 49 is a schematic diagram of a cross-section taken along line X1-X1' of the medical device shown in FIG. 48;

FIG. 50 is a partial enlarged view of a portion X11 in FIG. 49;

FIG. 51 is a schematic diagram illustrating a structure of a medical device during another operation process according to some embodiments of the present disclosure;

FIG. 52 is a schematic diagram of a cross-section taken along line X2-X2' of the medical device shown in FIG. 51;

FIG. 53 is a partial enlarged view of a portion X22 in FIG. 52;

FIG. 54 is a schematic diagram illustrating a structure of a medical device during another operation process according to some embodiments of the present disclosure;

FIG. 55 is a schematic diagram of a cross-section taken along line X3-X3' of the medical device shown in FIG. 54;

FIG. 56 is a partial enlarged view of a portion X33 in FIG. 55;

FIG. 57 is a schematic diagram illustrating a structure of a medical device during another operation process according to some embodiments of the present disclosure;

FIG. 58 is a schematic diagram of a cross-section taken along line X4-X4' of the medical device shown in FIG. 57;

FIG. 59 is a partial enlarged view of a portion X44 in FIG. 58;

FIG. 60 is schematic diagram illustrating a structure of a medical device according to some other embodiments of the present disclosure;

FIG. 61 is a schematic diagram illustrating a structure of a handle according to some further embodiments of the present disclosure;

FIG. 62 is a schematic diagram illustrating a structure of a delivery assembly according to some other embodiments of the present disclosure;

FIG. 63 is a schematic diagram illustrating a structure of a moving component being a clamp portion according to some embodiments of the present disclosure;

FIG. 64 is a schematic diagram illustrating a structure of a clamp jaw of the clamp portion of FIG. 63 according to some embodiments of the present disclosure;

FIGS. 65A and 65B are schematic diagrams each illustrating a structure of an intermediate piece of the clamp portion of FIG. 63 according to some embodiments of the present disclosure;

FIG. 66 is a schematic diagram illustrating a structure of a tightening tube of the clamp portion of FIG. 63 according to some embodiments of the present disclosure;

FIG. 67 is schematic diagram illustrating a structure of a clamp jaw and an intermediate piece in a closed state according to some embodiments of the present disclosure;

FIGS. 68A and 68B are schematic diagrams each illustrating a structure of a clamp portion during use according to some embodiments of the present disclosure;

FIG. 69 is a schematic diagram illustrating a structure of a clamp portion after use according to some embodiments of the present disclosure;

FIGS. 70A, 70B, and 70C are schematic diagrams each illustrating a structure of a guide groove according to some embodiments of the present disclosure;

FIG. 71 is a schematic diagram illustrating a structure of a first groove portion according to some embodiments of the present disclosure;

FIG. 72 is a schematic diagram illustrating a structure of a second groove portion according to some embodiments of the present disclosure;

FIG. 73 is a schematic diagram illustrating a structure of a support rod according to some other embodiments of the present disclosure;

FIG. 74 is a schematic diagram illustrating a structure of a rotary cap according to some other embodiments of the present disclosure;

FIG. 75 is a schematic diagram illustrating a structure of a handle according to some further embodiments of the present disclosure;

FIG. 76 is a schematic diagram illustrating a structure of a handle in a third gear according to some embodiments of the present disclosure;

FIG. 77 is a schematic diagram illustrating a structure of a handle in a third gear according to some other embodiments of the present disclosure;

FIG. 78 is a schematic diagram illustrating a structure of a handle according to some further embodiments of the present disclosure;

FIG. 79 is a schematic diagram illustrating a structure of a connector according to some other embodiments of the present disclosure;

FIG. 80 is a schematic diagram illustrating a structure of a handle according to some further embodiments of the present disclosure;

FIG. 81 is a schematic diagram illustrating a structure of a handheld portion according to some further embodiments of the present disclosure;

FIG. 82 is a schematic diagram illustrating a structure of a handle according to some further embodiments of the present disclosure;

FIG. 83 is a schematic diagram illustrating a structure of a handle in a second gear according to some embodiments of the present disclosure;

FIG. 84 is a schematic diagram illustrating a structure of a handle in a first gear according to some embodiments of the present disclosure;

FIG. 85 is a schematic diagram illustrating a structure of a handle in a third gear according to some other embodiments of the present disclosure;

FIG. 86 is a schematic diagram illustrating a structure of a handle in a second gear according to some other embodiments of the present disclosure;

FIG. 87 is a schematic diagram illustrating a structure of a handle in a first gear according to some other embodiments of the present disclosure.

### DETAILED DESCRIPTION

To more clearly illustrate the technical solutions in the embodiments of the present disclosure, the drawings used in the description of the embodiments will be briefly introduced below. Obviously, drawings described below are only some examples or embodiments of the present disclosure. Those skilled in the art, without further creative efforts, may apply the present disclosure to other similar scenarios according to these drawings. Unless obviously obtained from the context or the context illustrates otherwise, the same numeral in the drawings refers to the same structure or operation.

As shown in the present disclosure and the claims, unless the context clearly indicates an exception, the terms "a", "an", "one", and/or "the" do not specifically refer to the singular form and may also include the plural form. In general, the terms “comprise,” "comprises,” and/or “comprising,” “include,” “includes,” and/or “including,” merely prompt to include steps and elements that have been clearly identified, and these steps and elements do not constitute an exclusive listing. The methods or devices may also include other steps or elements. The term "based on" is "based at least in part on". The term "one embodiment" means "at least one embodiment"; the term "another embodiment" means "at least one other embodiment".

In the present disclosure, unless otherwise explicitly specified and limited, terms such as "install", "connect", "couple", and "fix" should be understood broadly. For example, a connection may be a fixed connection, a detachable connection, or an integral connection. A connection may be a mechanical connection, an electrical connection, or a direct connection. A connection may be an indirect connection through an intermediate medium, an internal communication between two elements, or an interaction relationship between two elements, unless otherwise explicitly limited. For those of ordinary skill in the art, the specific meanings of the above terms in the present disclosure may be understood based on specific situations.

It will be understood that the terms “system”, “engine”, “unit”, “module”, and/or “block” used herein are one method to distinguish different components, elements, parts, sections, or assemblies of different levels in ascending order. However, the terms may be displaced by other expressions if they may achieve the same purpose.

The terms "first", "second", and similar words used in the specification and claims of the present application do not denote any order, quantity, or importance, but are merely used to distinguish different components. Similarly, words such as "a" or "an" do not denote a quantity limitation, but denote the existence of at least one. In the description of the present disclosure, the meaning of "a plurality of" is at least two, such as two, three, etc., unless otherwise explicitly and specifically limited.

Unless otherwise indicated, terms such as "front", "rear", "lower", and/or "upper" are merely for convenience of description and are not limited to one position or one spatial orientation. In general, the terms "include" and "comprise" merely indicate the inclusion of explicitly identified steps and elements. These steps and elements do not constitute an exclusive list. A method or device may also include other steps or elements.

The flowcharts used in the present disclosure illustrate operations that systems implement according to some embodiments of the present disclosure. It is to be expressly understood, the operations of the flowcharts may be implemented not in order. Conversely, the operations may be implemented in an inverted order, or simultaneously. Moreover, one or more other operations may be added to the flowcharts. One or more operations may be removed from the flowcharts.

Embodiments of the present disclosure provide a handle for a medical device. The handle includes a handheld portion, a driving portion, and a plurality of transmission portions configured to be operatively coupled to a plurality of moving components at a distal end of the medical device. In some embodiments, the handheld portion is slidably engaged with the driving portion. An operator may actuate the driving portion to slide the driving portion relative to the handheld portion. In some embodiments, the driving portion is switchably connected to at least one of the plurality of transmission portions. In some embodiments, the driving portion is switchable between being connected to only one of the plurality of transmission portions and being simultaneously connected to at least two of the plurality of transmission portions.

In some embodiments, the driving portion is slid to a specific position to switch a connection of the driving portion from one of the plurality of transmission portions to another one of the plurality of transmission portions. In some embodiments, the plurality of transmission portions are connected one-to-one with the plurality of moving components at the distal end of the medical device. When the driving portion is switched to connect with one of the plurality of transmission portions, the driving portion may drive the plurality of moving components connected to the plurality of transmission portions to move.

In the embodiments of the present disclosure, the driving portion is configured to be switchably connected to at least one of the plurality of transmission portions in a corresponding manner, thereby enabling the driving portion to switchably drive at least one of the plurality of moving components to move. The configuration allows an operator to control the plurality of moving components by operating the handle, which increases safety when controlling the plurality of moving components and reduces operation errors.

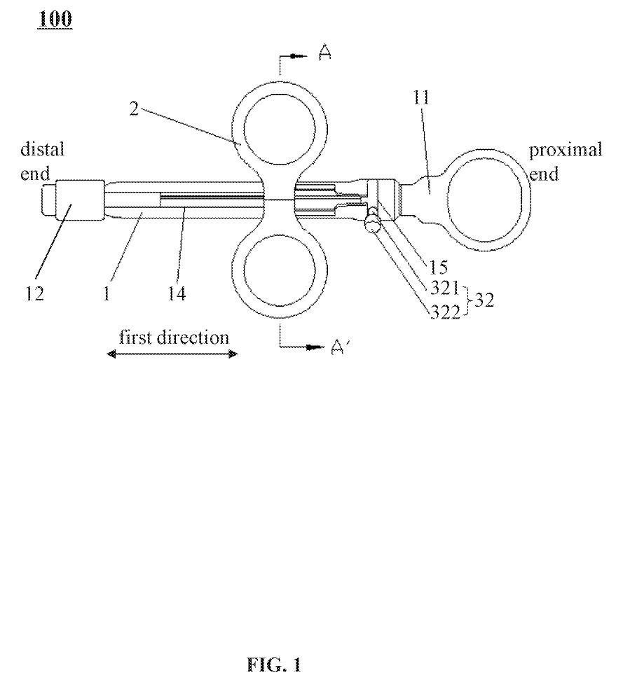

FIG. 1 is a schematic diagram illustrating a structure of a handle according to some embodiments of the present disclosure. As shown in FIG. 1, a handle 100 includes a handheld portion 1, a driving portion 2, and transmission portions.

A medical device is configured to perform diagnosis and treatment procedures. By operating the handle 100, an operator may control the medical device to implement diagnosis and treatment operations (e.g., grasping, electrocoagulation, cutting, sampling, etc.).

The handheld portion 1 refers to a portion of the handle 100 for an operator to hold. In some embodiments, the driving portion 2 and the transmission portions are movably disposed on the handheld portion 1. In some embodiments, the handheld portion 1 may be a structure of any shape that facilitates an operator to hold. In some embodiments, to facilitate an operator to hold the handle 100, the handheld portion 1 includes a proximal finger ring 11. The proximal finger ring 11 is disposed at a proximal end of the handheld portion 1. The proximal finger ring 11 may be used for an operator to hold with a single hand. In some embodiments, to facilitate an operator to operate the handle 100, the proximal finger ring 11 is rotatably mounted at the proximal end of the handheld portion 1. In other embodiments, the proximal finger ring 11 may also be fixed at the proximal end of the handheld portion 1. In some embodiments, the proximal finger ring 11 is detachably connected to the handheld portion 1. In some embodiments, to facilitate connection of the handle 100 to other structures of the medical device (e.g., a sheath tube), the handheld portion 1 further includes a connection member 12. In some embodiments, the connection member 12 is detachably mounted at a distal end of the handheld portion 1. It should be noted that the "proximal end" described in the present disclosure refers to an end of the handle 100 close to an operator. Correspondingly, the "distal end" refers to an end of the handle 100 away from the operator.

In some embodiments, the proximal finger ring 11 being rotatably connected at the proximal end of the handheld portion 1 may be achieved by a bearing structure, a damping hinge, or the like. The proximal finger ring 11 being fixing at the proximal end of the handheld portion 1 may be achieved by integral molding, welding, interference fit, or the like. Detachable connection between the proximal finger ring 11 and the handheld portion 1 or detachable mounting of the connection member 12 at the distal end of the handheld portion 1 may be achieved by threaded connection, snap-fit connection, magnetic attraction connection, or the like. Embodiments of the present disclosure impose no limitation thereon.

The driving portion 2 refers to a portion for an operator to drive a plurality of moving components at the distal end of the medical device. In some embodiments, the driving portion 2 is slidably engaged with the handheld portion 1. The driving portion 2 may slide relative to the handheld portion 1 along a first direction. The first direction is a sliding direction of a slidable engagement of the handheld portion 1 and the driving portion 2. The term “slidable engagement” refers to the driving portion 2 and the handheld portion 1 being assembled together, allowing relative movement (sliding) while maintaining a precise guiding relationship (sliding along the first direction) without random shaking. In some embodiments, the slidable engagement between the driving portion 2 and the handheld portion 1 may be achieved by a hole-shaft fit, a guide rail and a slider structure, or the like.

In some embodiments, the driving portion 2 may rotate relative to the handheld portion 1 around the first direction. In some embodiments, the driving portion 2 may first slide along the first direction and then rotate relative to the handheld portion 1 around the first direction. In some embodiments, the first direction may be an axial direction of the handle 100 or a direction approximately the axial direction. The axial direction of the handle 100 refers to an extension direction between the proximal end and the distal end of the handle 100. For ease of understanding, the following descriptions will use the first direction being the axial direction as an example. In some embodiments, the driving portion 2 first slides along the axial direction of the handle 100 and then rotates along a circumferential direction of the handle 100. The circumferential direction of the handle 100 refers to a direction surrounding the axial direction of the handle 100. In some embodiments, when the driving portion 2 slides to a preset position along the axial direction, the driving portion 2 is rotatable relative to the handheld portion 1 around the axial direction. The preset position refers to a position where the driving portion 2 and the transmission portions are switchable and connectable in the first direction.

In other embodiments, rotation of the driving portion 2 relative to the handheld portion 1 around the first direction is restricted. When the driving portion 2 slides to the preset position along the axial direction, the transmission portions are movable to connect with the driving portion 2. More descriptions regarding the driving portion 2 not being rotatable relative to the handheld portion 1 around the first direction may be found in the related descriptions below.

A moving component of the plurality of moving components refers to an end effector component that is driven and is configured to generate an action at the distal end of the medical device. For example, the moving component includes scissors, forceps, a cutter, a clamp portion, or the like. In some embodiments, the clamp portion is a three-prong hemostatic clamp. In some embodiments, the clamp portion may perform one or more medical operations such as clamping, electrocoagulation, cutting, sampling, or the like. In some embodiments, the plurality of moving components include a plurality of clamp jaws of the clamp portion. In some embodiments of the present disclosure, the moving component is described by taking the three-prong hemostatic clamp as an example.

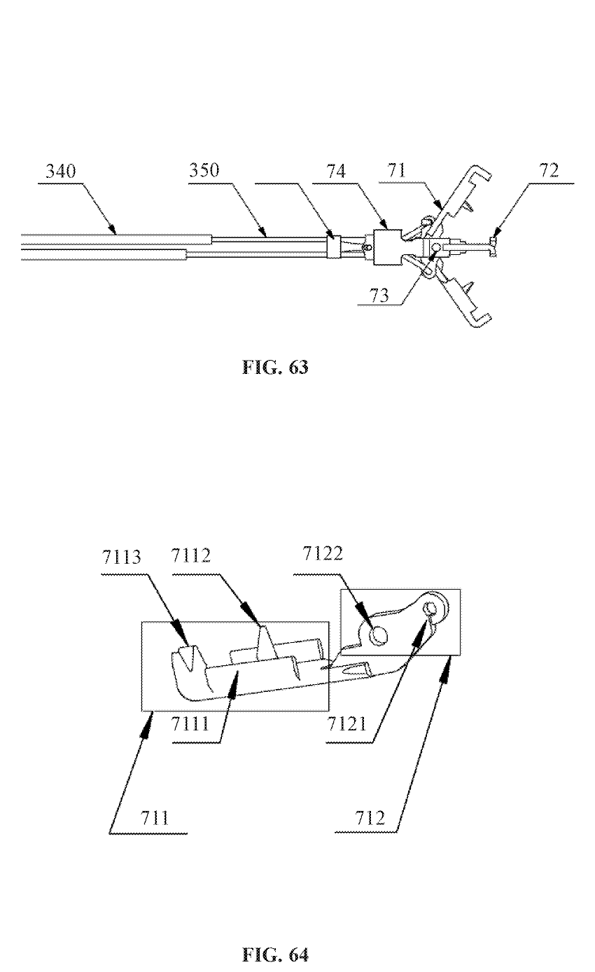

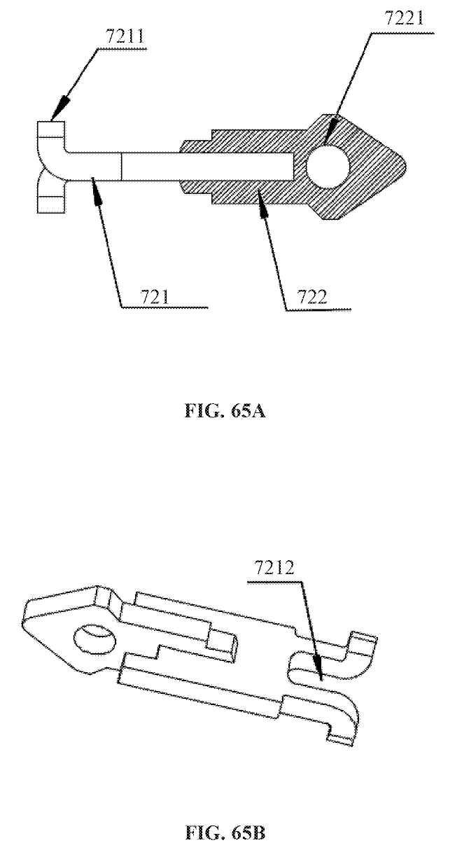

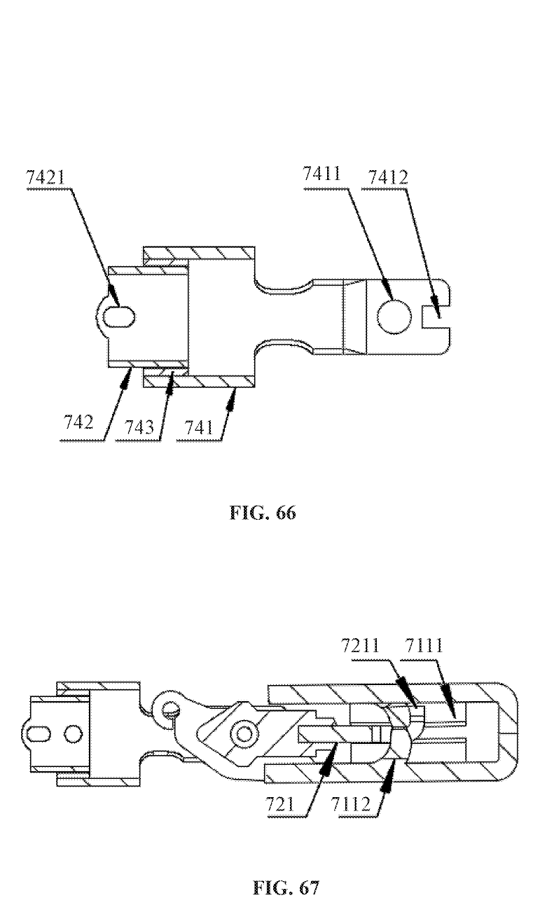

FIG. 63 is a schematic diagram illustrating a structure of a moving component being a clamp portion according to some embodiments of the present disclosure. FIG. 64 is a schematic diagram illustrating a structure of a clamp jaw of the clamp portion of FIG. 63 according to some embodiments of the present disclosure. FIGS. 65A and 65B are schematic diagrams each illustrating a structure of an intermediate piece of the clamp portion of FIG. 63 according to some embodiments of the present disclosure. FIG. 66 is a schematic diagram illustrating a structure of a tightening tube of the clamp portion of FIG. 63 according to some embodiments of the present disclosure.

In some embodiments, as shown in FIG. 63, a clamp portion includes a clamp jaw 71, an intermediate piece 72, a pin tube 73, and a tightening tube 74.

The clamp jaw 71 refers to a movable clamp jaw. In some embodiments, as shown in FIG. 63, the clamp portion includes two clamp jaws 71 and one intermediate piece 72. The two clamp jaws 71 are disposed on opposite sides of the intermediate piece 72 along a direction perpendicular to a first direction, respectively. The two clamp jaws 71 are each movably connected to the intermediate piece 72, and the two clamp jaws 71 are movable toward or away from each other relative to the intermediate piece 72 to clamp or release an object.

In some embodiments, as shown in FIG. 64, the clamp jaw 71 includes a clamp arm 711 and a clamp arm connecting piece 712.

The clamp arm 711 refers to a cantilever structure on the clamp jaw 71 for directly applying a clamping force. The clamp arm connecting piece 712 refers to a base or an elastic hinge structure for connecting the two clamp arms 711. In some embodiments, the clamp arm 711 and the clamp arm connecting piece 712 may be connected in various ways, such as welding, or the like.

The intermediate piece 72 refers to a fixed clamp jaw.

In some embodiments, as shown in FIG. 65A, the intermediate piece 72 includes an intermediate limiting piece 721 and a limiting gasket 722.

The intermediate limiting piece 721 refers to a component for separating the two clamp jaws 71. The intermediate limiting piece 721 provides a fixed support base to prevent misalignment or tilting of the two clamp jaws 71 during movement. When the clamp jaw 71 moves relative to or collides with the intermediate piece 72, the limiting gasket 722, as a soft isolation layer, absorbs impact, reduces noise, and provides lubricating feel. In some embodiments, the intermediate limiting piece 721 and the limiting gasket 722 may be connected in various ways, such as welding, or the like.

The pin tube 73 refers to a tubular part for providing a rotation fulcrum or a pin connection. The pin tube 73 passes through holes on the clamp jaw 71 and the intermediate piece 72 and serves as a rotation axis for opening and closing the clamp jaw 71. The clamp jaw 71 may rotate around the pin tube 73.

The tightening tube 74 refers to a driving component for applying a clamping force or a locking force to the clamp jaw.

In some embodiments, as shown in FIG. 66, the tightening tube 74 includes a base 741, a tube tail 742, and a sleeve tube 743.

The base 741 refers to a portion for directly contacting or connecting to the clamp jaw 71. The tube tail 742 refers to a tail end portion of the tightening tube 74 (an end away from the clamp jaw 71). The sleeve tube 743 refers to a tubular outer layer for cooperating with an internal channel of the handheld portion or the intermediate piece. The sleeve tube 743 provides a smooth guiding surface for axial movement of the tightening tube and protects an internal structure. In some embodiments, the base 741, the tube tail 742, and the sleeve tube 743 may be fixed in various ways, such as welding or the like, to form an integral whole. The pin tube 73 is coaxially fitted with the clamp jaw 71 and the intermediate piece 72. A transmission portion is hooked on a tail of the clamp arm connecting piece 712. When a first connection member drives the transmission portion to move, the transmission portion may drive the two clamp jaws 71 to repeatedly open and close.

As shown in FIG. 64, the clamp arm 711 includes a side wing wrapping structure 7111, a protrusion structure 7112, and a tooth tip 7113. The clamp arm connecting piece 712 includes a tail hook 7121 and a first hole 7122. As shown in FIG. 65A, the intermediate limiting piece 721 includes two distal teeth 7211. The limiting gasket 722 includes a second hole 7221. As shown in FIG. 65B, a gap 7212 is provided between the two distal teeth 7211, and the protrusion structure 7112 may pass through the gap 7212 between the two distal teeth 7211. As shown in FIG. 66, the base 741 includes a third hole 7411 and a groove 7412. The tube tail 742 includes a fourth hole 7421. After the clamp arm 711 and the clamp arm connecting piece 712 are connected (e.g., by welding or bonding), an integral clamp jaw 71 is formed. After the intermediate limiting piece 721 and the limiting gasket 722 are connected (e.g., by welding or bonding), an integral intermediate piece 72 is formed. After the base 741, the tube tail 742, and the sleeve tube 743 are welded, an integral tightening tube 74 is formed. The clamp jaw 71, the intermediate piece 72, and the tightening tube 74 are assembled by the pin tube 73 passing through the first hole 7122, the second hole 7221, and the third hole 7411 and then flaring. Simultaneously, the intermediate limiting piece 721 is engaged in the groove 7412 for limiting. The transmission portion hooks the tail hook 7121 to drive the clamp jaw 71 to rotate around the pin tube 73, thereby achieving repeated opening and closing of the clamp portion.

The transmission portions are configured to connect the driving portion 2 with the plurality of moving components at the distal end of the medical device. In some embodiments, the handle 100 includes a plurality of transmission portions. The plurality of transmission portions correspond one-to-one with the plurality of moving components. The plurality of transmission portions are located at different positions on the handle 100. In some embodiments, when the driving portion 2 slides to a corresponding position, the driving portion 2 may be connected with transmission portions at a corresponding position through an operation. The driving portion 2 drives the moving components connected to the transmission portions at the corresponding position to move, thereby achieving a corresponding diagnosis and treatment procedure. In some embodiments, the driving portion 2 has an operating state in which the driving portion 2 drives connected transmission portions to slide synchronously. When the driving portion 2 is connected to the transmission portions, an operator operates the driving portion 2 to slide, which drives the transmission portions to operate, thereby controlling designated moving components to operate. For example, an operation of the transmission portions controls the moving components to open, close, clamp tissue, perform sampling, or the like.

In some embodiments, the plurality of transmission portions are distributed around the first direction. In some embodiments, the driving portion 2 is rotatable relative to the handheld portion 1 around the first direction. Rotation of the plurality of transmission portions relative to the handheld portion 1 around the first direction is restricted. That is, the plurality of transmission portions are not freely rotatable around the first direction. The driving portion 2 is rotatable around the first direction at the preset position. The driving portion 2 is switchably connected to one or more of the plurality of transmission portions to actuate the moving components coupled to the connected transmission portions. In some embodiments, the driving portion 2 is rotatably connected to the handheld portion 1 around the first direction. When the driving portion 2 rotates to a position corresponding to any transmission portion around the first direction, the driving portion 2 is connectable with corresponding transmission portions. In some embodiments, the driving portion 2 is also slidably engaged with the handheld portion 1 in the first direction and rotatably engaged with the handheld portion 1 around the first direction. The driving portion 2 slides to the preset position along the first direction. When the driving portion 2 rotates relative to the handheld portion 1 to a position corresponding to any transmission portion around the first direction, the driving portion 2 is connectable with the corresponding transmission portions. In some embodiments, when the driving portion 2 is rotatable relative to the handheld portion 1 around the first direction, rotation of the plurality of transmission portions relative to the handle 100 around the first direction is restricted. For example, the plurality of transmission portions are not rotatable relative to the handle 100 around the first direction. In some embodiments, when rotation of the plurality of transmission portions relative to the handle 100 around the first direction is restricted, the driving portion 2 may include a plurality of interfaces distributed at intervals along a circumferential direction. The plurality of transmission portions are distributed at intervals along the circumferential direction. A distribution angle of the interfaces matches a distribution angle of the transmission portions. When the driving portion 2 rotates around the first direction to a corresponding position, the plurality of interfaces respectively and simultaneously dock with the plurality of transmission portions, thereby achieving synchronous connection between the driving portion and the plurality of transmission portions. When the driving portion rotates around the first direction to another corresponding position, the interfaces and the transmission portions are misaligned, and the driving portion is connected to only a single transmission portion.

In some embodiments, positions of the plurality of transmission portions on the handheld portion 1 are adjustable. By adjusting a position of a transmission portion, any one or more of the plurality of transmission portions are connectable with the driving portion 2. The driving portion 2 drives the movement of the moving components through the transmission portions. In some embodiments, the positions of the plurality of transmission portions are adjustable around the first direction. In the present embodiment, the driving portion 2 is slidably engaged with the handheld portion 1 in the first direction. Rotation of the driving portion 2 relative to the handheld portion 1 around the first direction is restricted. For example, the driving portion 2 is not rotatable relative to the handle 100 around the first direction. The driving portion 2 slides to the preset position along the first direction. Positions of the transmission portions around the first direction are adjusted to connect with the driving portion 2. In the present embodiment, the driving portion 2 is also slidably engaged with the handheld portion 1 in the first direction and rotatably engaged with the handheld portion 1 around the first direction. The driving portion 2 slides to the preset position along the first direction. The driving portion 2 rotates relative to the handheld portion 1 around the first direction, and/or the positions of the transmission portions around the first direction are adjusted, so that the driving portion 2 connects with the transmission portions. In some embodiments, the driving portion 2 is slidably engaged with the handheld portion 1 in the first direction. Positions of the plurality of transmission portions in a direction around the first direction are adjustable. By adjusting the positions of the transmission portions around the first direction, the driving portion 2 is controlled to slide to the preset position along the first direction, so that the driving portion 2 connects with the transmission portions. The driving portion 2 drives the movement of the moving components connected to the transmission portions. The transmission portions are connected with the driving portion 2. In some embodiments, the driving portion 2 and the transmission portions are arranged at intervals in the first direction, which may avoid an erroneous connection between the driving portion 2 and the transmission portions. Separating operations of the driving portion 2 and the transmission portions may largely avoid operation errors. In some embodiments, by adjusting positions of the transmission portions on the handheld portion 1 to dock with the driving portion, the driving portion 2 may switch between being connected to only one of the plurality of transmission portions and being simultaneously connected to at least two of the plurality of transmission portions.

In some embodiments, the handle 100 includes an adjustment portion. The adjustment portion cooperates with the plurality of transmission portions (e.g., slidably cooperates). The adjustment portion drives at least one of the plurality of transmission portions to be switchably connected to the driving portion 2. When the designated moving components need to operate, the driving portion 2 is driven to slide to the preset position, and the adjustment portion adjusts positions of different transmission portions, so that the transmission portions connected to the moving components are connected with the driving portion 2. An operator then drives the driving portion 2 to slide along the first direction, which may drive the transmission portions to operate, thereby controlling the designated moving components to operate. When switching to control another moving component is needed, the driving portion 2 and transmission portions connected to the driving portion 2 are driven to slide and return to the preset position. The adjustment portion then controls other transmission portions to be connected with the driving portion 2.

In some embodiments, the plurality of transmission portions includes a target transmission portion. The target transmission portion is engaged with the driving portion. When the driving portion is driven toward a distal end or a proximal end of the driving portion, the driving portion drives the target transmission portion to slide synchronously along the first direction, so as to drive at least one of the plurality of moving components connected to the target transmission portion of the plurality of moving components to perform an action.

The target transmission portion refers to a transmission portion selected to be engaged with the driving portion. The target transmission portion may be one transmission portion or the plurality of transmission portions. In some embodiments, the target transmission portion is connected and engaged with the driving portion 2 through a connection member. The connection member includes various types, such as a plug-in push-pull structure, a magnetic coupling structure, a snap-fit structure, or the like.

When the driving portion is driven toward the distal end or the proximal end of the driving portion, the driving portion 2 drives the target transmission portion to slide synchronously along the first direction, so as to drive the moving component connected to the target transmission portion of the plurality of moving components to perform an action.

In some embodiments, the handle 100 includes an adjustment portion. The adjustment portion cooperates with the plurality of transmission portions to drive at least one of the plurality of transmission portions to be switchably connected to the driving portion 2.

The adjustment portion refers to a portion for switching a transmission portion connected to the driving portion. In some embodiments, the adjustment portion may be a structure such as a shifting fork, a slider, an end-face cam, or the like.

Engagement between the adjustment portion and the transmission portion includes various types, such as sliding engagement, or the like. Sliding engagement between the adjustment portion and the transmission portion may be achieved in various ways, for example, a dovetail groove structure, a hole-shaft engagement structure, or the like. Taking the hole-shaft engagement structure as an example, a guide post is arranged on the transmission portion, and a corresponding through hole is arranged on the adjustment portion. The guide post is inserted into the through hole. By controlling a tiny gap between the guide post and the through hole, linear sliding is achieved.

When a specified moving component needs to be actuated, the driving portion 2 is driven to slide to the preset position. The adjustment portion is configured to adjust a position of a different transmission portion to cause the transmission portion connected to the moving component to connect to the driving portion 2. An operator then drives the driving portion 2 to slide along the first direction to drive the transmission portion to actuate, thereby controlling the specified moving component to actuate. When control needs to be switched to another moving component, the driving portion 2 and the transmission portion connected to the driving portion 2 are driven to slide and return to the preset position. The adjustment portion is then configured to control another transmission portion to connect to the driving portion 2.

In some embodiments, the adjustment portion rotates around the first direction to adjust the positions of the transmission portions.

In some embodiments, the adjustment portion is rotatably connected to the handheld portion 1. A rotation axis of a rotatable connection is parallel to the first direction. The adjustment portion is connected to the plurality of transmission portions. When the adjustment portion rotates relative to the handheld portion 1, the adjustment portion drives the plurality of transmission portions to rotate. Rotation of the plurality of transmission portions enables one or more of the plurality of transmission portions to switch connection with the driving portion 2.

In some embodiments, the adjustment portion is slidably engaged with the plurality of transmission portions along the first direction. In some embodiments, the adjustment portion is connected to one or more transmission portions. When the adjustment portion slides relative to the handheld portion 1 along the first direction, the adjustment portion drives the one or more transmission portions connected to the adjustment portion to slide along the first direction. After one or more transmission portions are connected with the driving portion 2, the driving portion 2 drives the one or more transmission portions connected to the driving portion 2 to slide together toward the distal end or the proximal end of the handle 100, so as to actuate the designated moving components.

In some embodiments, a transmission portion includes a transmission rod and a pull wire (not shown in the figures). The transmission rod is disposed inside the handheld portion 1. The transmission rod is connected to the moving components through the pull wire. The adjustment portion drives different transmission portions to be connected with the driving portion 2, and then drives different moving components to operate by driving different pull wires. In some embodiments, the driving portion 2 rotates around a circumferential direction of the handheld portion 1. Positions of the plurality of transmission portions remain fixed. The driving portion 2 rotates to be connected with the corresponding transmission portions. In this case, a position of a pull wire is not affected. The circumferential direction of the handheld portion 1 is parallel to the circumferential direction of the handle 100.

In some embodiments, the adjustment portion drives the plurality of transmission portions to rotate around the circumferential direction of the handheld portion 1 to select the corresponding transmission portions to be connected with the driving portion 2. In this case, the driving portion 2 does not need to rotate to be connected with different transmission portions. During a process of using the handle 100, a finger of an operator controlling the driving portion 2 only needs to apply an axial driving force to the driving portion 2 to operate the handle, and rotation of the adjustment portion may be completed by another hand of the operator. Thus, an operation difficulty of the handle may be reduced, and the controllability of the handle may be improved.

Following descriptions illustrate various implementations of the handle 100 with reference to the drawings. It should be noted that the following embodiments are merely illustrative and are not intended to limit the scope of the present disclosure. Those skilled in the art may make variations or adjustments based on the embodiments described in the present disclosure, and these variations or adjustments also fall within the scope of the present disclosure.

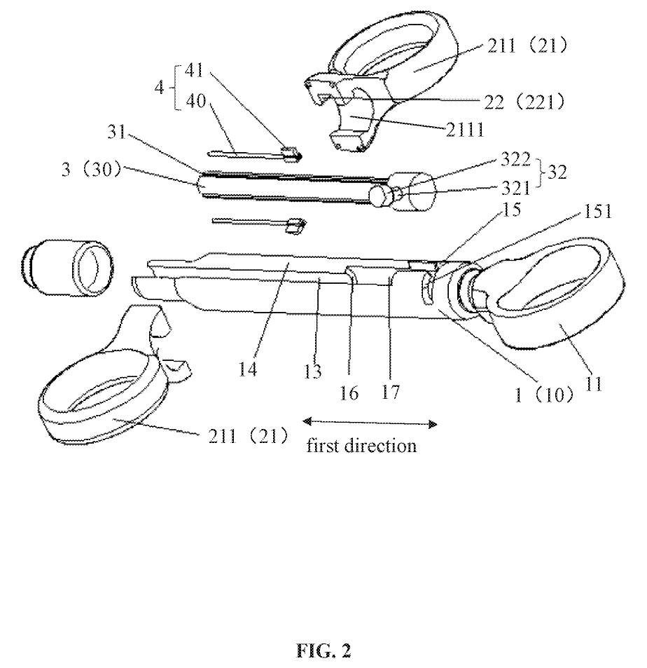

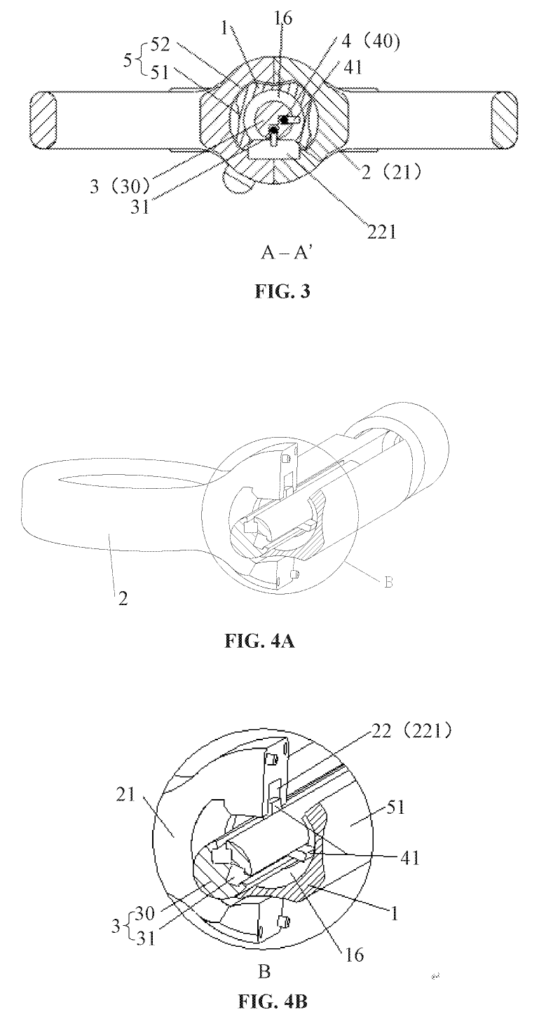

FIG. 2 is an exploded view of a handle according to some embodiments of the present disclosure. FIG. 3 is a schematic diagram of a cross-section taken along line A-A' in FIG. 1. FIG. 4A is a cross-sectional view of a connection between a driving portion and a transmission portion according to some embodiments of the present disclosure. FIG. 4B is an enlarged view of a portion B in FIG. 4A.

In some embodiments, as shown in FIG. 2, the handheld portion 1 includes a support housing 10. The support housing 10 encloses a mounting cavity 13. The support housing 10 is provided with a sliding guide groove 14 along the first direction. In some embodiments, the driving portion 2 is sleeved outside the handheld portion 1. The driving portion 2 is slidable along the first direction of the handheld portion 1. The support housing 10 is an outer structural member of the handheld portion 1.

In some embodiments, the driving portion 2 includes a sliding member 21. The sliding member 21 may be arranged in a detachable structure. As shown in FIG. 2, the sliding member 21 is formed by splicing two relatively arranged portions, which facilitates installation of the driving portion 2 on the handheld portion 1. The two detachable portions of the sliding member 21 form an insertion channel for passing through the handheld portion 1, so that the driving portion 2 is sleeved outside the handheld portion 1. In some embodiments, the sliding member 21 may be symmetrically split into two portions along a direction parallel to the first direction. Splicing of the two split portions of the sliding member 21 may be implemented in various ways, for example, snap-fit splicing, threaded splicing, or the like.

In some embodiments, the sliding member 21 includes a sliding finger ring 211. A finger of an operator is inserted into the sliding finger ring 211 to operate the driving portion 2. Specifically, each of the two detachable portions of the sliding finger ring 211 is provided with a notch 2111. An inner side of the notch 2111 is adapted to an outer side of the handheld portion 1, so that the driving portion 2 may be clamped on the outer side of the handheld portion 1 through the notch 2111. The notches 2111 on the two detachable portions of the sliding member 21 are spliced to form the insertion channel. In some embodiments, the sliding member 21 may also be configured as an integrally formed structure. The insertion channel is disposed in a middle portion of the sliding member 21. The insertion channel is configured for the handheld portion 1 to pass through, enabling the driving portion 2 to slide along an axial direction of the handheld portion 1.

In some embodiments, with reference to FIG. 3, the driving portion 2 is sleeved outside the handheld portion 1 through the notch 2111, and a limiting structure 5 is disposed between the driving portion 2 and the handheld portion 1 to restrict rotation of the driving portion 2. The limiting structure 5 includes a first mating surface 51 disposed on the handheld portion 1 and a second mating surface 52 disposed on the driving portion 2. The first mating surface 51 and the second mating surface 52 cooperate with each other to form an irregular curved surface, thereby restricting the rotation of the driving portion 2 around the first direction.

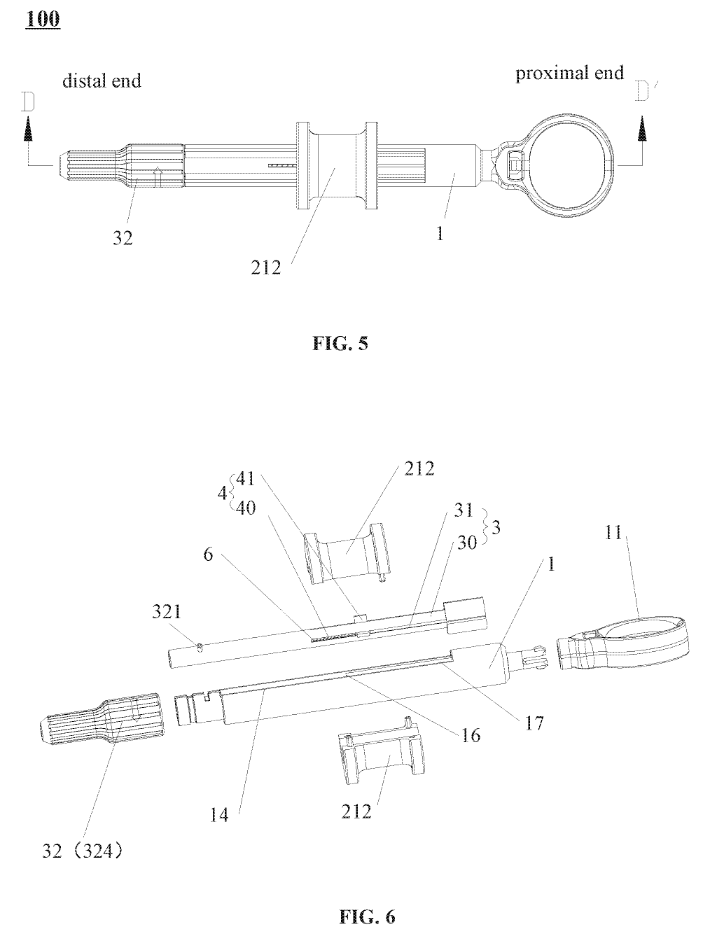

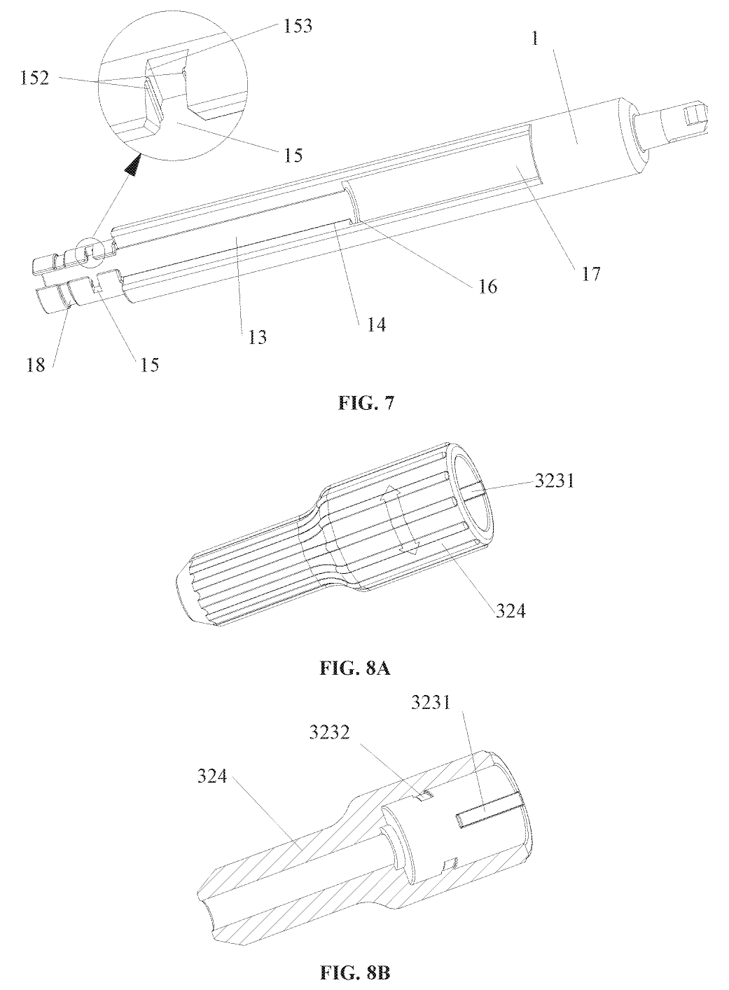

FIG. 5 is a schematic diagram illustrating a structure of a handle according to some other embodiments of the present disclosure. FIG. 6 is an exploded view of a handle according to some embodiments of the present disclosure. FIG. 7 is a schematic diagram illustrating a structure of a handheld portion according to some embodiments of the present disclosure. FIG. 8A is a schematic diagram illustrating a structure of a rotary cap according to some embodiments of the present disclosure. FIG. 8B is a cross-sectional view of FIG. 8A.

Differences between the handle 100 shown in FIGS. 5-6 and the handle 100 shown in FIGS. 2-4B include that a sliding member 21 includes an annular ring 212. The annular ring 212 is an intermediate connection component for slidably mounting the driving portion 2 on the handheld portion 1.

In some embodiments, referring to FIGS. 5-6, for ease of installation, the annular ring 212 is configured as a split structure, so that the annular ring 212 is detachably mounted on the handheld portion 1. A groove (not shown in FIGS. 5-6) included in the driving portion 2 is disposed on the annular ring 212. In some embodiments, to facilitate an operator to push the annular ring 212 by hand, a proximal end and a distal end of the annular ring 212 may be provided with a stopper or a retaining ring to form a groove for hand gripping.

In some embodiments, referring to FIGS. 2-6, an adjustment portion 3 is rotatably installed within a mounting cavity 13. The adjustment portion 3 rotates around a rotation axis of the adjustment portion 3. The rotation axis is an axis of the adjustment portion 3 itself and parallel to the first direction. When the driving portion 2 and transmission portions 4 are both located at a preset position in the first direction, an operator or an operating device may drive the adjustment portion 3 to rotate to switch the transmission portions 4 connected to the driving portion 2.

In some embodiments, the adjustment portion 3 includes a support rod 30. The support rod 30 is rotatably installed within the mounting cavity 13. The rotation axis refers to a central axis of the support rod 30 itself. In some embodiments, the support rod 30 is provided with a plurality of sliding grooves 31 along the first direction. The plurality of sliding grooves 31 are disposed in one-to-one correspondence with the plurality of transmission portions 4. Each of a plurality of transmission portions 4 is slidably engaged with the adjustment portion 3 through one of the plurality of sliding grooves 31.

In some embodiments, the plurality of sliding grooves 31 all penetrate an end surface at a distal end of the support rod 30, so that the transmission portions 4 may pass through the support rod 30 to be connected with a plurality of moving components. In some embodiments, the plurality of sliding grooves 31 are stepped to prevent the plurality of transmission portions 4 from disengaging from the plurality of sliding grooves 31 along a radial direction of the support rod 30. The radial direction of the support rod 30 refers to a direction on a cross-section perpendicular to the rotation axis of the support rod 30, the direction pointing from a center of the cross-section perpendicularly to an outside of the support rod 30.

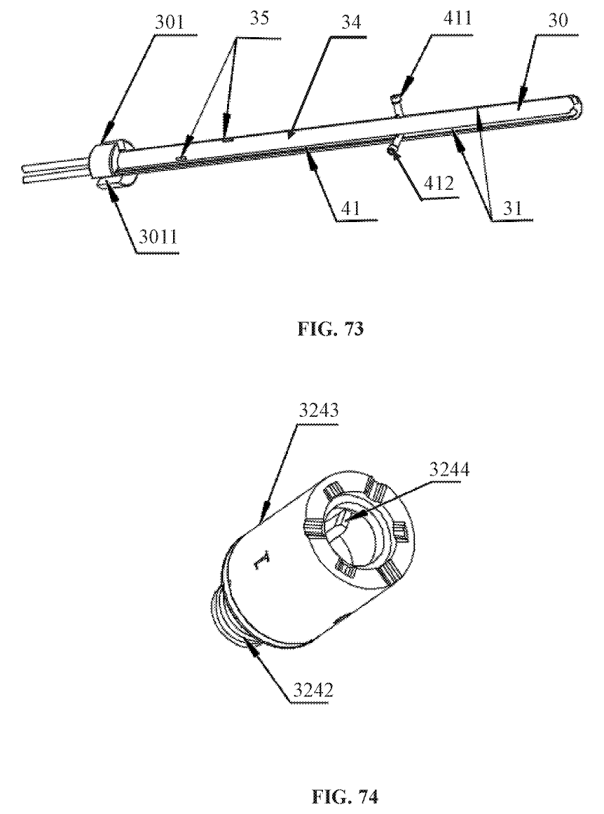

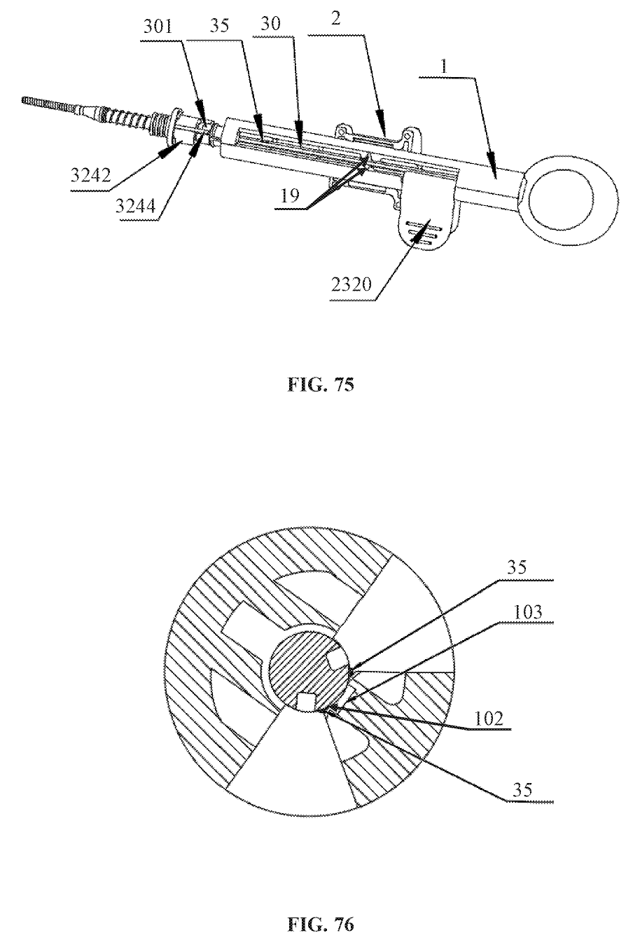

FIG. 73 is a schematic diagram illustrating a structure of a support rod according to some other embodiments of the present disclosure. FIG. 75 is a schematic diagram illustrating a structure of a handle according to some further embodiments of the present disclosure. FIG. 76 is a schematic diagram illustrating a structure of a handle in a third gear according to some embodiments of the present disclosure.

In some embodiments, as shown in FIGS. 73, 75, and 76, the support rod 30 includes a rod body 34 and a support protrusion 35. The support protrusion 35 is disposed on an outer surface of the rod body 34.

The support protrusion 35 refers to a protruding structure on the rod body 34. In some embodiments, the support protrusion 35 may be a portion of the rod body 34 and integrally formed with the rod body 34. The support protrusion 35 may also be a separate component fixed to the rod body 34 by gluing, welding, or the like. In some embodiments, the support protrusion 35 may be a lever 321 fixedly connected to the support rod 30 as described below.

In some embodiments, a volume of the support rod 30 is less than an internal space volume of the mounting cavity 13 of the handheld portion, facilitating installation of the support rod 30 within the mounting cavity 13 of the handheld portion. In some embodiments, since the volume of the support rod 30 is less than the internal space volume of the mounting cavity 13 of the handheld portion, the support protrusion 35 may be disposed on the outer surface of the support rod 30 to abut against an inner wall of the mounting cavity 13. Interaction between the support protrusion and the handheld portion restricts wobbling of the first connection member when opening and closing in a third gear, so as to reduce wobbling caused by size inconsistency during use.

A gear refers to different operating positions or modes in which a specific component (e.g., the handheld portion, the first connection member, the rotary cap, etc.) is located. Each gear corresponds to a specific connection state between the transmission portion and the driving portion, for example, a first connection state, a second connection state, a third connection state, etc., described below. The first connection state, the second connection state, and the third connection state respectively represent specific cooperation relationships achieved by internal mechanisms of the handle 100 in corresponding gears. To avoid ambiguity, when referring to "a component being in an X-th gear ", it means the component is operated to or located at a predetermined position corresponding to the "X-th gear ". At this time, an interior of the mechanism presents a connection state corresponding to the gear.

In some embodiments, the gears include a first gear, a second gear, and a third gear. The first gear corresponds to the third connection state, the second gear corresponds to the second connection state, and the third gear corresponds to the first connection state.

More descriptions regarding the gears may be found in the related descriptions below.

In some embodiments, referring to FIGS. 2 and 76, the handheld portion 1 includes the support housing 10. A support wall 102 and an avoidance groove 103 are disposed on an inner side of the support housing 10.

The support wall 102 refers to a wall surface on the inner side of the support housing 10 for abutting against the support protrusion 35. In some embodiments, the support protrusion 35 on the support rod 30 abuts against the support wall 102 during normal operation, effectively eliminating a gap between the rod body 34 and the support housing 10.

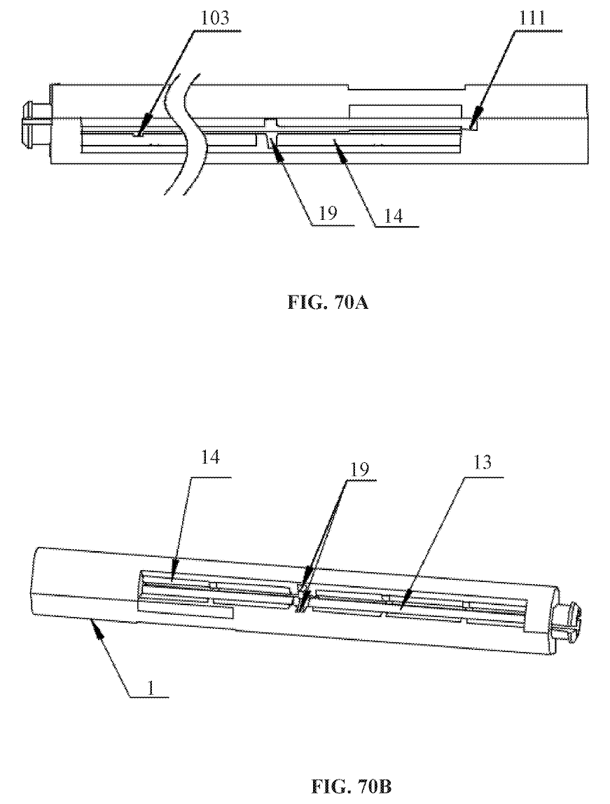

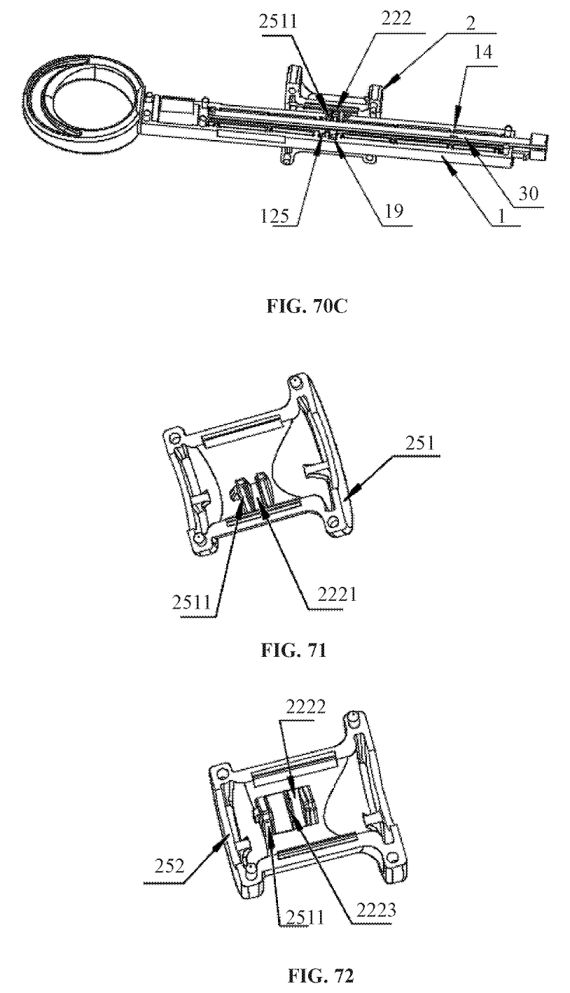

The avoidance groove 103 refers to a cavity in the support housing 10 that provides temporary space for the support protrusion 35 to temporarily disengage from abutting against the support wall 102. In some embodiments, the avoidance groove 103 may be disposed at a plurality of positions. For example, as shown in FIG. 70A, the avoidance groove 103 is disposed on an inner side of the handheld portion 1 near the support protrusion 35. In some embodiments, a radial depth of the avoidance groove 103 is greater than a protrusion height of the support protrusion 35, so that the support protrusion 35 may enter the avoidance groove 103.

In some embodiments, as shown in FIG. 76, when a rotary cap 324 is in the third gear, the support protrusion 35 on the support rod 30 is at two ends of the avoidance groove 103, limiting shaking of the support rod 30 during opening and closing of a clamp portion. When gear shifting is performed, the rotary cap 324 is rotated, and the support protrusion 35 passes over a side wall of the avoidance groove 103 and may enter the avoidance groove 103.

In some embodiments, the adjustment portion 3 includes a rotation driving assembly 32. The rotation driving assembly 32 is configured to drive the support rod 30 to rotate around the rotation axis when subjected to an external force. In some embodiments, the rotation driving assembly 32 is connected and disposed at a proximal end or a distal end of the support rod 30. Certainly, in other embodiments, the rotation driving assembly 32 may also be located at a middle portion between the proximal end and the distal end of the support rod 30. In some embodiments, for ease of operation by an operator, the rotation driving assembly 32 is located at the proximal end of the handheld portion 1.

In some embodiments, referring to FIGS. 2-4B, the rotation driving assembly 32 includes a toggle member. The toggle member includes a lever 321. The handheld portion 1 includes a lever guide groove 15. The lever guide groove 15 is disposed on the handheld portion 1. One end of the lever 321 is fixedly connected to the support rod 30, and another end of the lever 321 passes through the lever guide groove 15 and extends outside the handheld portion 1. An operator may operate an end of the lever 321 located outside the handheld portion 1 to cause the support rod 30 to rotate, thereby achieving rotation of the adjustment portion 3 within the mounting cavity 13. In some embodiments, the lever guide groove 15 is arranged around the first direction to cause the support rod 30 to rotate around the rotation axis.

In some embodiments, to prevent the lever 321 from rotating arbitrarily and to enable the lever 321 to move to a designated position, the lever guide groove 15 includes one or more positioning notches 151. The positioning notches 151 are disposed within the lever guide groove 15. The lever 321 moves along the lever guide groove 15 to drive the plurality of transmission portions 4 to rotate around the rotation axis. When the lever 321 is engaged with one of the positioning notches 151, the driving portion 2 is connected to one of the plurality of transmission portions 4. In some embodiments, the positioning notch 151 may be a protrusion structure or a recess structure. The protrusion structure protrudes from a wall surface of the lever guide groove 15. The recess structure is recessed relative to the wall surface of the lever guide groove 15. Both the protrusion structure and the recess structure may be used to position the lever 321.

In some embodiments, to facilitate an operator to operate the lever 321 while also limiting the adjustment portion 3 to prevent the lever 321 from disengaging from the lever guide groove 15, an end of the lever 321 located outside the lever guide groove 15 is connected to a toggle member 322. An edge of the toggle member 322 exceeds the lever guide groove 15 and is limited outside the lever guide groove 15, which may prevent the lever 321 from disengaging from the lever guide groove 15. In some embodiments, the toggle member 322 may be configured as a circular block, an arc-shaped block, or the like. In some embodiments, a side surface of the toggle member 322 is attached to a partial wall surface of the handheld portion 1.

In some embodiments, referring to FIGS. 5-8B, the rotation driving assembly 32 includes a rotary cap 324. In some embodiments, to increase the comfort of hand gripping for an operator and facilitate the operator to operate the handle 100 with both hands, the rotation driving assembly 32 is located at a distal end of the handle 100. The rotation driving assembly 32 is connected to the support rod 30 and is rotatably connected to the handheld portion 1 to drive the support rod 30 to rotate around an axis of the support rod 30. In some embodiments, the rotation driving assembly 32 includes the rotary cap 324. As shown in FIGS. 8A and 8B, the rotary cap 324 is configured as an integral annular structure. The rotary cap 324 is sleeved over the distal end of the handheld portion 1 and is rotatably connected to the handheld portion 1. Specifically, as shown in FIGS. 6-7, the lever guide groove 15 is disposed at the distal end of the handheld portion 1. The lever 321 is fixedly installed at the distal end of the support rod 30, passes through the lever guide groove 15 to be engaged with a matching groove 3231 located on the rotary cap 324, so that the rotary cap 324 may drive the support rod 30 to rotate. A stop protrusion 152 protruding within the lever guide groove 15 may form a stop groove 153 at an end of the lever guide groove 15 to limit a rotation stroke of the support rod 30.

In some embodiments, a locking groove 18 is disposed at the distal end of the handheld portion 1. A locking ring 3232 is disposed on an inner wall surface of the rotary cap 324. When the rotary cap 324 is sleeved onto the distal end of the handheld portion 1, the locking ring 3232 is snapped into the locking groove 18 to form a rotational fit. For ease of assembly, the matching groove 3231 is disposed on the inner wall surface of the rotary cap 324. The matching groove 3231 extends from a proximal end to the distal end of the rotary cap 324, so that the lever 321 may directly slide into the matching groove 3231 during assembly of the rotary cap 324. It can be understood that the lever 321 fixedly connected to the support rod 30 forms a limit stop radially protruding outward from the support rod 30. The matching groove 3231 forms a groove disposed axially on the rotary cap 324.

FIG. 74 is a schematic diagram illustrating a structure of a rotary cap according to some other embodiments of the present disclosure.

In some embodiments, as shown in FIG. 73, a connection platform 301 is provided at a distal end of the support rod 30. The connection platform 301 is connected to the rotary cap 324.

The connection platform 301 refers to a component for connecting the support rod and the rotary cap.

The connection between the connection platform 301 and the rotary cap 324 may be achieved in various ways, for example, via a key connection, or the like.

In some embodiments, as shown in FIG. 74, the rotary cap 324 includes a connection hub 3242 and a rotary sleeve 3243. The rotary sleeve 3243 is fixed on an outer side of the connection hub 3242. A plurality of keys 3244 are formed on an inner side of the connection hub 3242 located inside the rotary sleeve 3243.

The connection hub 3242 refers to a base or a hub located inside the rotary cap for connecting to an external component (e.g., the support rod or the adjustment portion) to transmit rotational torque outward. The rotary sleeve 3243 refers to an outer shell or operating surface sleeved on the outer side of the connection hub for an operator to rotate. The key 3244 refers to a protrusion structure disposed on an inner side of the connection hub.

In some embodiments, a keyway 3011 is provided on the connection platform 301 for the key 3244 to be embedded in. In some embodiments, when the keyway 3011 is engaged with the key 3244, the support rod 30 is connected to the rotary cap, thereby enabling the rotary cap 324 to drive the support rod 30 to rotate together when the rotary cap 324 rotates.

In some embodiments, as shown in FIG. 73, the support rod 30 is provided with a pair of sliding grooves 31 along a first direction. The first connection member is embedded in the sliding groove 31 and may move along the sliding groove 31. A connector may be inserted into the sliding groove 31. When the support rod 30 rotates, the support rod 30 drives the first connection member to rotate together, enabling the connector to rotate within the sliding groove 31.

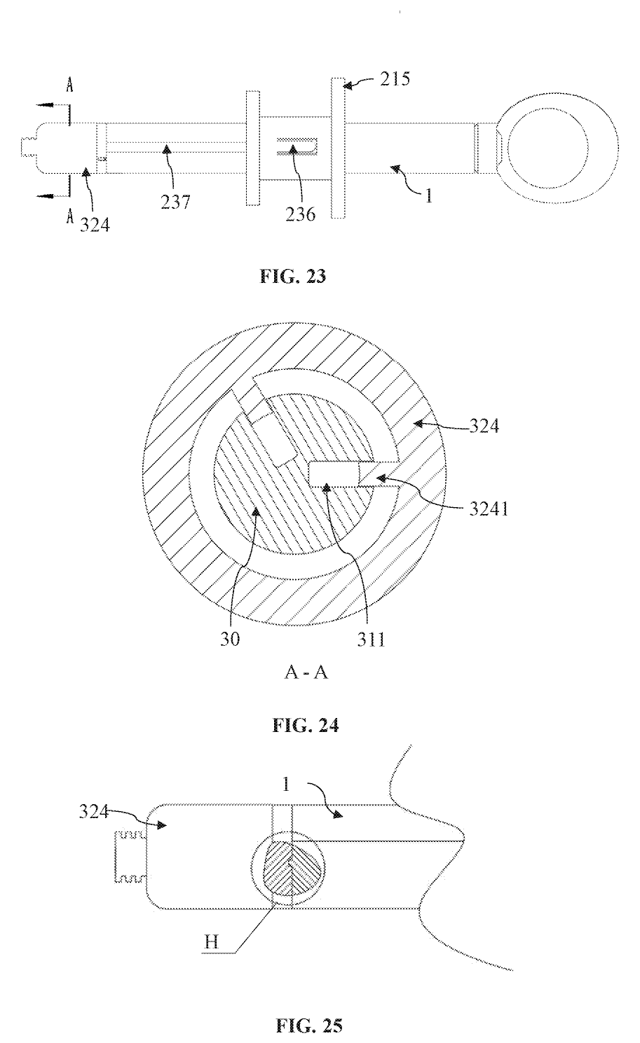

FIG. 23 is a schematic diagram illustrating a structure of a handle according to still some other embodiments of the present disclosure. In some embodiments, as shown in FIG. 23, the rotation driving assembly 32 includes a rotary cap 324. The rotary cap 324 is rotatably connected to the handheld portion 1. The rotary cap 324 is slidably engaged with the adjustment portion 3 along an axial direction of the adjustment portion 3. By rotating the rotary cap 324, the transmission portions 4 connected to the driving portion 2 may be switched.

FIG. 24 is a schematic diagram of a cross-section taken along line A-A of the handle shown in FIG. 23.

In some embodiments, to increase the comfort of hand gripping for an operator, the rotary cap 324 is located at the distal end of the handle 100. The rotary cap 324 is rotatably engaged around an axial direction of the handheld portion 1 and is connected to the support rod 30 to drive the support rod 30 to rotate around the rotation axis of the support rod 30. In some embodiments, the rotary cap 324 is sleeved on an outer side of a distal portion of the handheld portion 1 and is rotatably engaged with an outer side surface of the handheld portion 1.

In some embodiments, as shown in FIG. 24, the support rod 30 of the adjustment portion 3 includes a limiting groove 311 arranged along the axial direction. The limiting groove 311 is disposed toward the out side surface of the handheld portion 1. The rotary cap 324 includes a limit stop 3241 protruding radially inward relative to the support rod 30. The limit stop 3241 may at least partially extend into the limiting groove 311 and be slidably engaged with the limiting groove 311 along the first direction. When the limit stop 3241 is engaged within the limiting groove 311, the support rod 30 rotates along with the rotary cap 324, thereby switching the transmission portions 4 connected to the driving portion 2. In some embodiments, a plurality of limit stops 3241 and a plurality of limiting grooves 311 may be disposed in a one-to-one correspondence. It should be understood that the arrangement of the limit stop 3241 and the limiting groove 311 may be interchanged. For example, the support rod 30 includes a limit stop protruding radially outward, and the rotary cap includes a groove arranged along the axial direction. The limit stop at least partially extends into the groove and is slidably engaged with the groove.

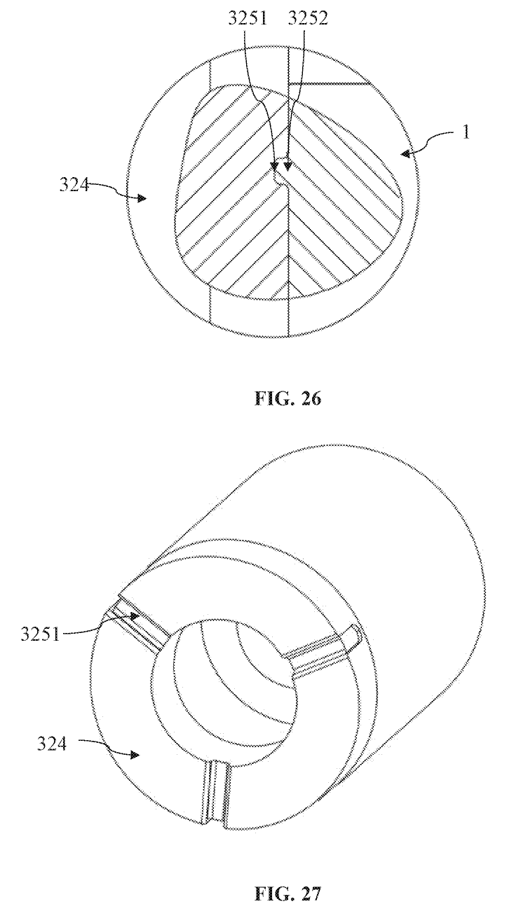

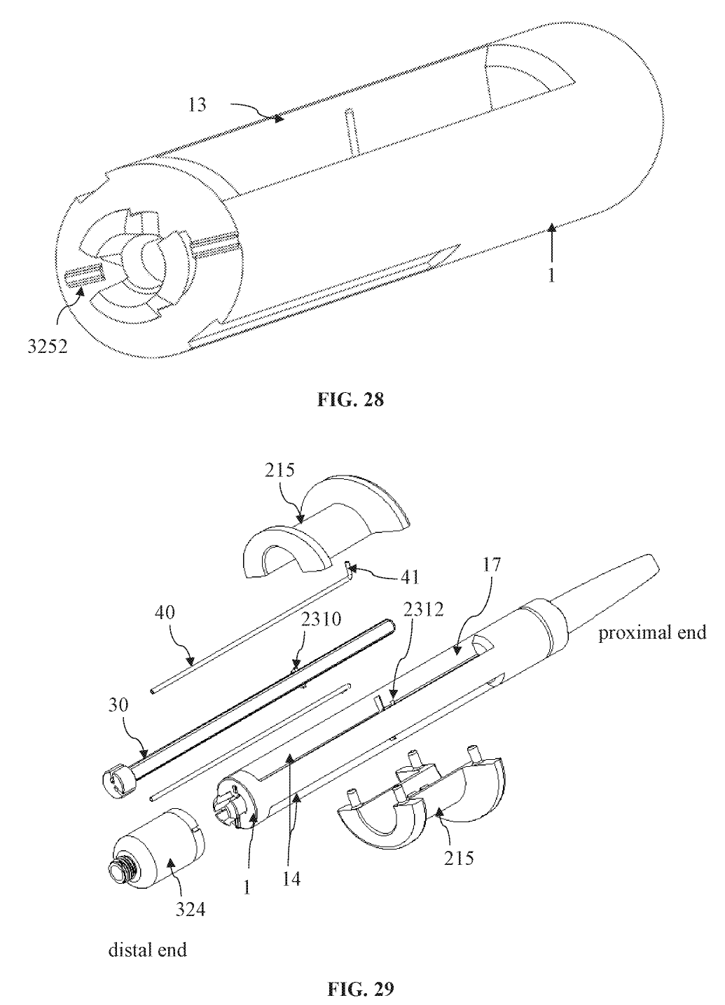

FIG. 25 is a schematic diagram of a connection between a rotary cap and a handheld portion according to some embodiments of the present disclosure. FIG. 26 is an enlarged view of a portion H in FIG. 25. FIG. 27 is a schematic diagram illustrating a structure of a locking groove according to some embodiments of the present disclosure. FIG. 28 is a schematic diagram illustrating a structure of a locking block according to some embodiments of the present disclosure.

In some embodiments, as shown in FIGS. 26-28, a gear configuration for positioning a rotation angle of the adjustment portion 3 is disposed between the rotary cap 324 of the adjustment portion 3 and the handheld portion 1. The gear configuration includes a locking groove 3251 and a locking block 3252 that are engaged with each other. A plurality of locking grooves 3251 are disposed on the rotary cap 324. The locking block 3252 is disposed on the handheld portion 1. The locking groove 3251 and the locking block 3252 are disposed close to each other, and the locking groove 3251 is opened toward the locking block 3252. In some embodiments, the locking groove 3251 is disposed on a side surface of the rotary cap 324 that connects with the handheld portion 1. The locking block 3252 is disposed on a side surface of the handheld portion 1 that connects with the rotary cap 324. The locking groove 3251 is arranged along a radial direction of the rotary cap 324. The locking block 3252 is arranged along a radial direction of the handheld portion 1 to conform to a rotation of the rotary cap 324. In some embodiments, the locking groove 3251 penetrates an outer side surface of the rotary cap 324 along the radial direction of the rotary cap 324. The locking block 3252 penetrates an outer side surface of the handheld portion 1 along the radial direction of the handheld portion 1, so that an operator may observe an engagement state between the locking groove 3251 and the locking block 3252. When the rotary cap 324 is rotated and the locking groove 3251 is engaged with the locking block 3252, the support rod 30 is rotated to a specified position. At this time, the driving portion 2 may be connected to corresponding transmission portions 4. It should be understood that the arrangement of the locking groove 3251 and the locking block 3252 may be interchanged. For example, a plurality of locking blocks 3252 are disposed on the rotary cap 324, and the locking groove 3251 is disposed on the handheld portion 1. The specified position refers to a position of the support rod when the locking groove and the locking block are engaged. The specified position may be selected by an operator. The radial direction of the rotary cap 324 refers to a direction that is perpendicular to a rotation axis of the rotary cap 324 and radiates outward from a rotation center of the rotary cap 324 as a circle center. The radial direction of the handheld portion 1 refers to a direction that is perpendicular to a central axis of the handheld portion 1 and radiates outward based on the central axis.

In some embodiments, the gear configuration includes a plurality of gear markings. The gear markings are disposed on an outer wall surface of the rotary cap 324 and correspond one-to-one with different gears formed by the locking groove 3251 and the locking block 3252.

In some embodiments, referring to FIGS. 2 and 6, a transmission portion 4 is disposed in the sliding groove 31 and is slidably engaged with the sliding groove 31. In some embodiments, the transmission portion 4 includes a transmission member 40. The transmission member 40 is disposed in the sliding groove 31 and is slidably engaged with the sliding groove 31. In some embodiments, the transmission portion 4 is connected to the driving portion 2 through connection members disposed between each other.

In some embodiments, at least some of a plurality of transmission portions 4 include a first connection member. The first connection member is fixedly connected to the transmission member 40 (e.g., a transmission rod) and protrudes from an outer wall surface of the support rod 30. Specifically, the sliding groove 31 is an open groove. A side portion of the sliding groove 31 is in communication with the outer wall surface of the support rod 30, so that the first connection member may protrude from the support rod 30.

In some embodiments, the driving portion 2 includes one or more second connection members 22. The one or more second connection members 22 are slidably engaged with the handheld portion 1 through the sliding guide groove 14. The one or more second connection members 22 are connectably engaged with the first connection member located on the transmission portions 4. When the first connection member and the one or more second connection members 22 are both located at a preset position in the first direction, and the adjustment portion 3 drives the transmission member 40 to rotate relative to the handheld portion 1 around the rotation axis, at least one first connection member is switched to be engaged with the one or more second connection members 22, so that at least one of the plurality of transmission portions 4 is connected to the driving portion 2.

In some embodiments, the first connection member is a connection protrusion, and the second connection member 22 is a connection recess. Alternatively, the first connection member is the connection recess, and the second connection member 22 is the connection protrusion. The connection protrusion and the connection recess are engaged in a snap-fit manner. In some embodiments, the connection protrusion may be a protrusion structure. The connection recess may be a groove structure. The protrusion structure may be snapped into the groove structure.

In some embodiments, when both the driving portion 2 and the first connection member are located at the preset position in the first direction, the adjustment portion 3 is drivable to switch the transmission portion 4 connected to the driving portion 2.

When both the driving portion 2 and the first connection member are located at the preset position in the first direction, both the first connection member and the one or more second connection members are located at the preset position in the first direction. The adjustment portion 3 drives the transmission rod 40 to rotate relative to the handheld portion 1 about the rotation axis, switches at least one first connection member to cooperate with the one or more second connection members 22, so that at least one of the plurality of transmission portions 4 is connected to the driving portion 2.

It should be noted that the "preset position" described in the present application refers to a state in which each component (e.g., the driving portion 2, the first connection member) moves relative to the handheld portion 1 to a predetermined spatial coordinate region. The predetermined spatial coordinate region is not an absolute spatial coordinate position (because a handle may be used anywhere), but is a position region relative to the handheld portion.

The driving portion 2 being located at the preset position refers to the driving portion 2 sliding along the first direction to an axial position corresponding to a locking member on the handheld portion 1. An accommodating space (e.g., an axial limiting space) for accommodating the first connection member is arranged at the axial position corresponding to the locking member on the handheld portion 1.

The first connection member being located at the preset position refers to the first connection member rotating with the transmission rod 40 to a circumferential position that is radially opposite to the second connection member on the driving portion 2.

Both the driving portion 2 and the first connection member being located at the preset position in the first direction refers to the driving portion 2 and the first connection member being aligned with the accommodating space along an axial position in three-dimensional space to achieve snap-fit.

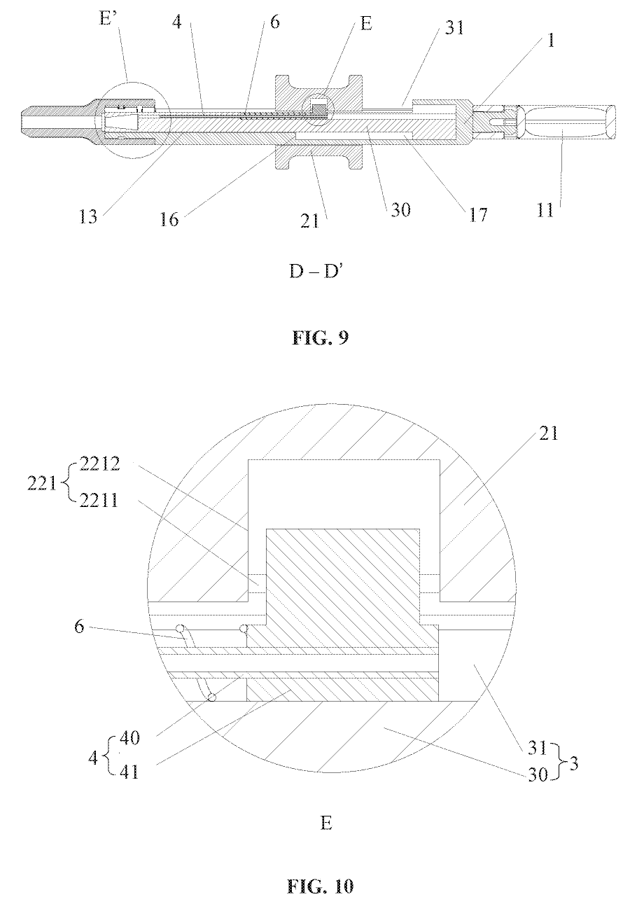

Taking a groove 221 as the second connection member disposed on the driving portion 2 and a protrusion 41 as the first connection member disposed on the transmission member 40 as an example, referring to FIGS. 2 and 3, when a sliding member 21 is configured as a detachable structure, the groove 221 may be simultaneously disposed on both detachable portions of the sliding member 21. Groove openings on the two portions of the sliding member 21 are in communication with each other and may be spliced to form a complete groove 221. When the sliding member 21 is integrally formed, the groove 221 is formed on an inner wall surface of the sliding member 21. In some embodiments, the groove 221 has opening ends 2211 (referring to FIG. 10). The opening ends 2211 are disposed at both ends of the groove 221 (i.e., the connection recess) around the rotation axis and have open mouths, so that the protrusion 41 may rotate into or out of the groove 221 as the adjustment portion 3 rotates. In some embodiments, the groove 221 includes blocking surfaces 2212 (referring to FIG. 10) disposed on both sides along the rotation axis for limiting a sliding movement of the transmission portions 4 relative to the driving portion 2. In some embodiments, the protrusion 41 is disposed at a proximal end of the transmission member 40, which allows the transmission member 40 to have a sufficiently long pushing distance within a certain size, avoiding an increase in an overall size of the handle due to an excessive overall length of the transmission member 40, and also preventing a driving effect of the transmission member 40 on the moving components due to an excessively short pushing distance. Certainly, in other embodiments, the protrusion 41 may also be located at a middle section, a distal end, or other positions of the transmission member 40.

FIG. 69 is a schematic diagram illustrating a structure of a clamp portion after use according to some embodiments of the present disclosure. FIGS. 70A, 70B, and 70C are schematic diagrams each illustrating a structure of a guide groove according to some embodiments of the present disclosure.

In some embodiments, as shown in FIGS. 70A-70C, the one or more second connection member includes a guide groove 222 extending along a circumferential direction of the handheld portion 1. After at least one first connection member enters the guide groove 222, the driving portion 2 is engaged with a corresponding transmission portion.

The guide groove 222 refers to a groove-shaped track for receiving and accommodating the at least one first connection member. In some embodiments, the guide groove 222 is arranged on the driving portion 2 and extends along the circumferential direction of the handheld portion 1. In some embodiments, a count of the guide groove 222 may be one or more.

FIG. 71 is a schematic diagram illustrating a structure of a first groove portion according to some embodiments of the present disclosure. FIG. 72 is a schematic diagram illustrating a structure of a second groove portion according to some embodiments of the present disclosure.

In some embodiments, as shown in FIGS. 71 and 72, the guide groove 222 includes a first groove portion 2221 and a second groove portion 2222. The first groove portion 2221 and the second groove portion 2222 are in communication with each other.

The first groove portion 2221 and the second groove portion 2222 refer to components of the guide groove 222.

In some embodiments, groove walls of the first groove portion 2221 and the second groove portion 2222 are connected at a junction to achieve communication. In some embodiments, the groove walls of the first groove portion 2221 and the second groove portion 2222 are disconnected at the junction. However, a communication cavity is arranged in a disconnection region. Two ends of the communication cavity communicate with the first groove portion 2221 and the second groove portion 2222, respectively.

In some embodiments, as shown in FIGS. 71 and 72, the driving portion 2 includes a first half-shell 251 and a second half-shell 252 connected to each other. The first groove portion 2221 is disposed in the first half-shell 251. The second groove portion 2222 is disposed in the second half-shell 252.

The first half-shell 251 and the second half-shell 252 refer to two shell portions of the driving portion arranged in a split manner. In some embodiments, the first half-shell 251 and the second half-shell 252 may be two opposite portions of a sliding member 21.

In some embodiments, as shown in FIG. 69, the handheld portion 1 is provided with two guide grooves 14 along an axial direction of the handheld portion 1. In some embodiments, as shown in FIGS. 70C, 71, and 72, the first half-shell 251 has two sliding bosses 2511 configured to be embedded in one of the guide grooves 14. An inner side of the two sliding bosses 2511 forms the first groove portion 2221. Correspondingly, the second half-shell 252 has the two sliding bosses 2511 configured to be embedded in another one of the guide grooves 14. The inner side of the two sliding bosses 2511 forms the second groove portion 2222. When the two half-shells are assembled together, the first groove portion 2221 and the second groove portion 2222 communicate at a joint position to form a complete guide groove 222. An assembly manner of the two half-shells is the same as a splicing manner of two split portions of the sliding member, and more descriptions may be found in the foregoing description.

In some embodiments, widths of the first groove portion 2221 and the second groove portion 2222 along the first direction are different. For example, as shown in FIGS. 71 and 72, a width of the first groove portion 2221 along the first direction is less than a width of the second groove portion 2222 along the first direction.

In some embodiments, when the driving portion is simultaneously connected to two transmission portions, the widths of the first groove portion 2221 and the second groove portion 2222 along the first direction are set to be different, so that the two transmission portions sequentially reach corresponding distal walls of the groove portions when driven by the driving portion.

In some embodiments, as shown in FIG. 72, the second groove portion 2222 includes a first accommodation chamber and a second accommodation chamber distributed along the first direction. A destructible barrier 2223 is disposed between the first accommodation chamber and the second accommodation chamber.

The first accommodation chamber and the second accommodation chamber refer to cavity regions in the second groove portion that are respectively close to a proximal end and a distal end of the handle.

The term "destructible" refers to a state change of the barrier 2223 under a specific condition. For example, the barrier 2223 is deformed under excessive pulling force. The specific condition may be set based on actual requirements. The barrier 2223 refers to a structural isolation element disposed between the first accommodation chamber and the second accommodation chamber. For example, the barrier 2223 is an elastic cantilever beam, a compressible protrusion, or the like. In some embodiments, taking the barrier 2223 as an elastic cantilever beam structure as an example, a root of the barrier 2223 is connected to a side wall of the guide groove, and a free end of the barrier 2223 extends into the guide groove. An outer periphery of the first connection member is provided with an annular groove corresponding to the barrier. In a normal state, the free end of the barrier is engaged in the annular groove of the transmission portion to lock the transmission portion at a preset position in the guide groove, preventing the transmission portion from moving accidentally due to vibration or unintended contact. When the transmission portion needs to be released, an operator applies a release force to the transmission portion along the first direction. The transmission portion moves along the guide groove, and an edge of the annular groove of the transmission portion pushes against the free end of the barrier, causing a cantilever beam to bend elastically, and the barrier disengages from the annular groove. The transmission portion may then continue to move to a release position. After the release is completed, the barrier elastically returns to an initial state, waiting for a next locking operation. Overload release is achieved through elastic deformation of the barrier, avoiding problems such as jamming of the transmission portion or damage to a release mechanism due to excessive release force, which would prevent release.

In some embodiments, after the at least one first connection member enters the guide groove 222, the driving portion 2 forms a fit with the corresponding transmission portion.

FIG. 78 is a schematic diagram illustrating a structure of a handle according to some further embodiments of the present disclosure. FIG. 79 is a schematic diagram illustrating a structure of a connector according to some other embodiments of the present disclosure.

In some embodiments, as shown in FIG. 73, the plurality of transmission portions include a first transmission portion and a second transmission portion. A first connection member corresponding to the first transmission portion includes a first connector 411. A first connection member corresponding to the second transmission portion includes a second connector 412. The first connector 411 and the second connector 412 are spaced apart along a circumferential direction of the handheld portion. A connection state between the plurality of transmission portions and the driving portion includes a first connection state. The first connection state is that the first connector 411 is connected to the driving portion 2 through the first groove portion 2221, and the second connector 412 is connected to the driving portion 2 through the second groove portion 2222.

The first transmission portion and the second transmission portion refer to the transmission portions for connecting different moving components.

In some embodiments, as shown in FIGS. 78 and 79, proximal ends of the first connection members are bent to form connectors (including the first connector 411 and the second connector 412). A bending angle may be set according to actual requirements, for example, 90°.

The first connector and the second connector being spaced apart along the circumferential direction of the handheld portion means that the two connectors are located at different positions in the circumferential direction of the handheld portion, for example, separated left and right, separated front and back, separated diagonally, or the like.

In some embodiments, when the plurality of transmission portions and the driving portion are in the first connection state, the first connector 411 and the second connector 412 are on opposite sides of the handheld portion 1. The first connector 411 is connected to the driving portion 2 through the first groove portion 2221. The second connector 412 is connected to the driving portion 2 through the second groove portion 2222. During release, the first connector first abuts against a distal wall of the first groove portion to achieve pull-off. The second connector subsequently abuts against a distal wall of the second groove portion to achieve sequential pull-off.

Taking a moving component as a clamp portion as an example, the first transmission portion and the second transmission portion are respectively connected to different clamp jaws of the clamp portion. When in the first connection state, the first connector 411 is connected to the driving portion 2 through the first groove portion 2221, and the second connector 412 is connected to the driving portion 2 through the second groove portion 2222. Pulling the driving portion can drive both the clamp jaws to move.

In some embodiments, as shown in FIG. 79, at least one of the first connector 411 and the second connector 412 includes a slip ring 413 and a connecting post 414. The slip ring 413 is sleeved outside the connecting post 414 and is rotatable relative to the connecting post 414.

The slip ring 413 refers to a rotatable component on the connector. The connecting post 414 refers to a fixed part of the connector.

In some embodiments, the connecting post 414 is fixed on the first connector or the second connector. Fixing may be achieved in various ways, for example, by integral molding, adhesive bonding, or the like.

In some embodiments, when an inner diameter of the slip ring 413 is greater than an outer diameter of the connecting post 414, and a difference between the inner diameter of the slip ring 413 and the outer diameter of the connecting post 414 is within a first preset range, the slip ring 413 is considered to be sleeved outside the connecting post 414. The first preset range may be set according to actual requirements.

In some embodiments, when a shape of the first connector or the second connector is cylindrical and a surface of the first connector or the second connector is sufficiently smooth, the first connector or the second connector may include only the slip ring, and the slip ring is sleeved outside the first connector or the second connector. The term "sufficiently smooth" means that the first connector or the second connector may support the slip ring to roll on an outer surface of the first connector or the second connector.

In some embodiments of the present disclosure, by providing the slip ring and the connecting post, friction between the first connection member and the driving portion and friction between the first connection member and the handheld portion are changed from sliding friction to rolling friction, reducing a friction force and making rotation and gear shifting of an adjustment portion smoother.

In some embodiments, a width of the guide groove is greater than a diameter of the slip ring, so that the slip ring may enter the guide groove, and the slip ring can slide within the guide groove.

In some embodiments, a difference between the width of the guide groove and the diameter of the slip ring is within a second preset range. The second preset range may be set based on actual requirements. For example, the second preset range is 0.1 mm to 0.5 mm.

In some embodiments of the present disclosure, by setting the width of the guide groove to be greater than the diameter of the slip ring, the slip ring and side walls of the guide groove do not form a clamping contact, which reduces movement resistance, avoids direct wear between the connector and the groove walls, and extends a service life.

In some embodiments, as shown in FIG. 75, the handheld portion 1 further includes at least one axial limiting space 19. The at least one axial limiting space 19 is in communication with at least one of the first groove portion 2221 and the second groove portion 2222.

The at least one axial limiting space 19 refers to a cavity region disposed in an axial direction of the handheld portion. In some embodiments, the at least one axial limiting space 19 is an arc-shaped cavity extending along a circumferential direction. Two ends of the at least one axial limiting space 19 are in communication with the first groove portion 2221 and the second groove portion 2222, respectively. An axial width of the at least one axial limiting space matches an axial dimension of the connector. The connector is axially limited and circumferentially slidable after entering the at least one axial limiting space.

In some embodiments, as shown in FIG. 75, the handheld portion 1 includes two axial limiting spaces 19 arranged at intervals. The two axial limiting spaces are in communication with at least one of the first groove portion 2221 and the second groove portion 2222, respectively.

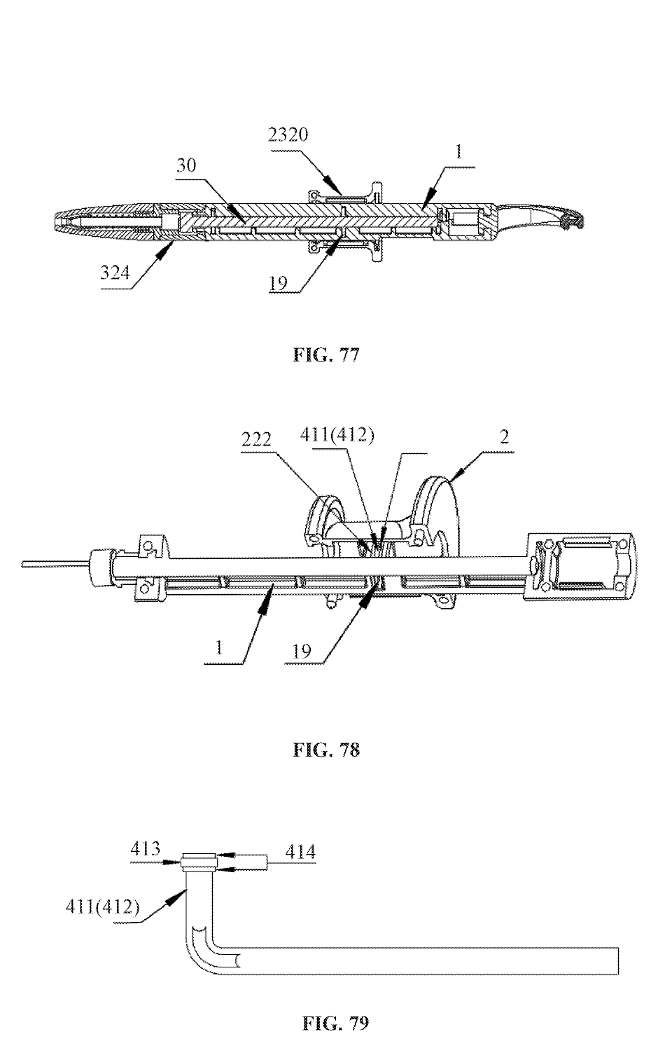

FIG. 77 is a schematic diagram illustrating a structure of a handle in a third gear according to some other embodiments of the present disclosure.

In some embodiments, with reference to FIGS. 70C and 77, when the driving portion 2 is located at the preset position, the guide groove 222 and one of the axial limiting spaces 19 combine to form a complete annular groove. At this time, the connector can rotate along the annular groove under the drive of the rotary cap 324, thereby causing the connector 181 to switch between the guide groove 222 and one of the axial limiting spaces 19. At this position, as shown in FIGS. 70C and 77, the guide groove 222 in the driving portion 2 aligns with one of the axial limiting spaces 19 in the handheld portion 1.

When the driving portion 2 leaves the preset position, the guide groove 222 and one of the axial limiting spaces 19 are misaligned. When the connector is located in the guide groove 222, the connector may move forward and backward following the driving portion 2 to open and close a clamp jaw connected to the connector. When the connector is located in one of the axial limiting spaces 19, the connector is prevented from moving forward and backward following the driving portion 2, thereby keeping the clamp jaw in a closed state.

In some embodiments, the connection state between the plurality of transmission portions and the driving portion further includes at least one of a second connection state and a third connection state. The second connection state is that the first connector is connected to the driving portion through the second groove portion, and the second connector is located in the at least one axial limiting space. The third connection state is that the second connector is connected to the driving portion through the first groove portion, and the first connector is located in the at least one axial limiting space.

In some embodiments, two ends of the at least one axial limiting space are in communication with the first groove portion and the second groove portion, respectively. When the transmission portion and the driving portion are in the first connection state and the driving portion rotates around the first direction, the first connector enters the second groove portion from the first groove portion, and the second connector enters the one of the axial limiting spaces from the second groove portion. At this time, the transmission portion and the driving portion are in the second connection state. After continuing to rotate, the first connector enters the one of the axial limiting spaces from the second groove portion, and the second connector enters the first groove portion from the one of the axial limiting spaces. At this time, the transmission portion and the driving portion are in the third connection state. Taking the moving component as a clamp portion as an example, in both the second connection state and the third connection state, only one connector is in the guide groove, and the other connector is in the one of the axial limiting spaces. At this time, pulling the driving portion may only drive one clamp jaw to move.

In some embodiments, when a position of the transmission portion may be changed on the handheld portion, the connection state between the plurality of transmission portions and the driving portion may further include a fourth connection state, that is, the first connection head and the second connection head are respectively located in the two axial limiting spaces.

In some embodiments, the handheld portion 1 includes a positioning portion 16. The positioning portion 16 is used for positioning the protrusion 41 at the preset position. Specifically, the positioning portion 16 may position a protrusion 41 that is not connected to the driving portion 2 at the preset position in the first direction. The preset position refers to a position where the protrusion 41 and the groove 221 are connected in the first direction.

In some embodiments, the positioning portion 16 is disposed in the mounting cavity 13 and forms a boss extending inward. The positioning portion 16 has at least a notch corresponding to the sliding guide groove 14. A protrusion 41 connected to the groove 221 may continue to slide along the first direction in the sliding groove 31 through the notch. It should be understood that the positioning portion 16 is not limited to forming the boss extending inward. For example, the positioning portion 16 includes a tube member disposed in the mounting cavity 13 of the handheld portion 1. An end surface of the tube member may position the protrusion 41 at the preset position in the first direction. In some embodiments, the positioning portion 16 is provided with notches corresponding one-to-one with the plurality of sliding guide grooves 14.

In some embodiments, the positioning portion 16 is disposed on a distal end of the protrusion 41. The positioning portion 16 is disposed toward a proximal end of the handle 100. When the distal end of the protrusion 41 abuts against the positioning portion 16, the protrusion 41 is located at the preset position. In some embodiments, the positioning portion 16 is disposed on a proximal end of the protrusion 41. When the proximal end of the protrusion 41 abuts against the positioning portion 16, the protrusion 41 is located at the preset position. A plurality of protrusions 41 are disposed on a same plane perpendicular to the first direction. When the protrusions 41 are at the preset position, one side end surface of each of the plurality of protrusions 41 is attached to one side end surface of the positioning portion 16, so that all the protrusions 41 are located on the same plane. A position in the first direction of a protrusion 41 not connected to the driving portion 2 is limited to the preset position by the positioning portion 16. When the driving portion 2 reaches the preset position, and when it is necessary to switch different protrusions 41 to be engaged with the groove 221, the protrusion 41 may be rotated by the adjustment portion 3.

In some embodiments, the handle 100 further includes an elastic reset member. The elastic reset member is elastic. A count of elastic reset members may be the same as a count of transmission portions. That is, one elastic reset member is provided corresponding to one transmission portion. When the elastic reset member is in a natural state, the first connection member (i.e., the protrusion 41) is located at the preset position. After the first connection member leaves the preset position, the elastic reset member provides a restoring force to return the first connection member to the preset position. The natural state refers to a state where the elastic reset member is not deformed, or a state where the elastic reset member does not generate an elastic restoring force. In some embodiments, the elastic reset member may be located on a distal end of the preset position. When the transmission portions 4 move toward the distal end or the proximal end, the elastic reset member generates the restoring force. The restoring force is directed toward the preset position. In some embodiments, the elastic reset member may be located on a proximal end of the preset position. When the transmission portions 4 move toward the distal end or the proximal end, the elastic reset member generates the restoring force. The restoring force is directed toward the preset position. In some embodiments, one end of the elastic reset member is connected to the transmission portions 4, and another end of the elastic reset member is connected to the adjustment portion 3, so as to provide the elastic restoring force to the transmission portions 4. By providing the elastic reset member, the first connection member not connected to the second connection member may always be maintained at the preset position.

In some embodiments, the elastic reset member may also provide the restoring force to return to the preset position for the first connection member already connected to the second connection member. After an operator operates the driving portion 2 to leave the preset position and then releases the driving portion 2, the driving portion 2 and the transmission portions 4 may return to the preset position under an action of the elastic reset member. More descriptions regarding the elastic reset member may be found in FIGS. 9-10 and the relevant descriptions thereof.

FIG. 9 is schematic diagram of a cross-section taken along line D-D' in FIG. 5. FIG. 10 is an enlarged view of a portion E shown in FIG. 9.

In some embodiments, referring to FIGS. 9 and 10, the handle 100 further includes an elastic reset member 6. One end of the elastic reset member 6 is connected to a transmission portion 4, and another end of the elastic reset member 6 is connected to the adjustment portion 3.

In some embodiments, the elastic reset member 6 is configured as a spring. The spring is arranged around the transmission member 40 (for example, sleeved on an outer side of the transmission member 40) to make a force on the transmission portions 4 more uniform. The elastic reset member 6 may be attached to a wall surface of the sliding groove 31 to avoid a radial force on the elastic reset member 6, which is beneficial for ensuring an elastic restoring effect of the elastic reset member 6. A distal end of the elastic reset member 6 is fixedly connected to a distal wall surface of the sliding groove 31. A proximal end of the elastic reset member 6 is fixedly connected to the protrusion 41.

It should be noted that the elastic reset member 6 and the aforementioned positioning portion 16 may be provided alternatively. Either the elastic reset member 6 or the positioning portion 16 may position the first connection member (for example, the protrusion 41) at the preset position. The elastic reset member 6 and the aforementioned positioning portion 16 may also be provided simultaneously, which may make positioning more precise. In some embodiments, when the elastic reset member 6 is in a natural state, the protrusion 41 is located at the preset position. To further improve stability of the positioning of the protrusion 41, the positioning portion 16 capable of abutting against the protrusion 41 may be provided to facilitate positioning of the protrusion 41.

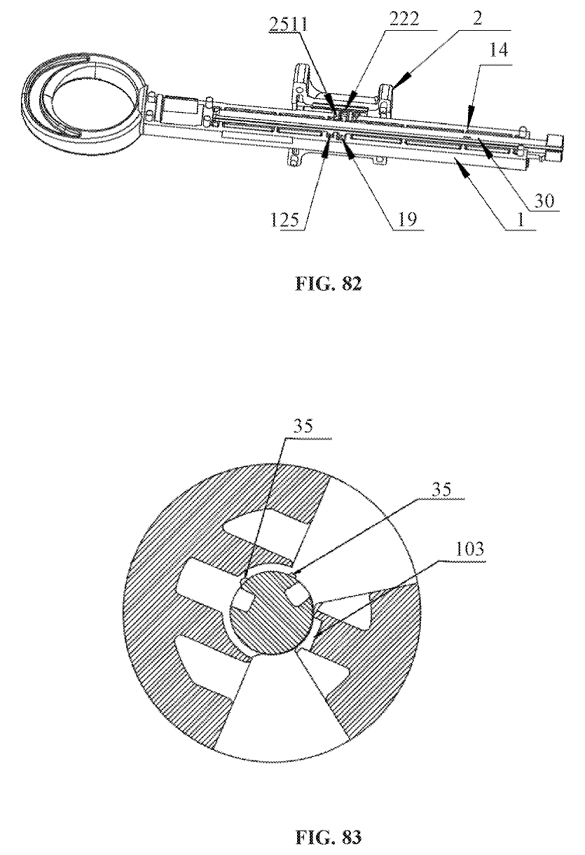

FIG. 81 is a schematic diagram illustrating a structure of a handheld portion according to some further embodiments of the present disclosure. FIG. 82 is a schematic diagram illustrating a structure of a handle according to some further embodiments of the present disclosure.

In some embodiments, as shown in FIGS. 81 and 82, a first positioning assembly includes a positioning tab. A positioning groove 125 is provided in the handheld portion 1 proximal to one of the axial limiting spaces 19. A deformable or breakable positioning tab is disposed in the positioning groove 125. When the driving portion 2 is at the preset position, the positioning tab may block a proximal end of the sliding boss 2511, ensuring that the clamp jaw may be repeatedly opened and closed, and gear shifting is completed when a limit position is reached. During release, the driving portion 2 continues to be pulled toward the proximal end. When the driving portion 2 moves toward the proximal end, the driving portion 2 may deform or break the positioning tab, so that the driving portion 2 may continue to move toward the proximal end to complete the release.

FIG. 11 is an enlarged view of a portion E' shown in FIG. 9.

In some embodiments, referring to FIG. 11, the adjustment portion 3 further includes a guide cavity 33 located at a distal end of the support rod 30. The guide cavity 33 has a diameter gradually decreased from a proximal end to the distal end of the support rod 30 to form an arc-shaped conical surface 331 that guides the distal end of the transmission portions 4 to extend toward a central axis of the support rod 30. Therefore, after the transmission portions 4 extend into the guide cavity 33, the transmission portions 4 are engaged with the arc-shaped conical surface 331 to gradually move closer to a central position of the guide cavity 33, so that the transmission portions 4 extend out from an outlet end of the guide cavity 33 (i.e., a port with a smaller diameter). The arrangement facilitates transmission of a force from the transmission portions 4 to the moving components to control the operation of the moving components.

In some embodiments, the handheld portion 1 includes a marking region (not shown in the figures). When the driving portion 2 moves to the marking region, the driving portion 2 and the first connection member cooperating with the driving portion 2 are located at the preset position. In some embodiments, the marking region may be provided on a surface of the handheld portion 1. The marking region has features different from other regions of the surface of the handheld portion 1. For example, the marking region has a color, a pattern, or a structure (for example, a protruding structure, a recessed structure, or a concave-convex structure) different from the other regions of the surface of the handheld portion 1 to prompt an operator to switch operations, i.e., to prompt the operator to switch from operating the driving portion 2 to operating the adjustment portion 3 to achieve switching of the first connection member connected to the driving portion 2.

In some embodiments, an additional positioning assembly may be further provided to position the driving portion 2 along the first direction, so that the driving portion 2 may accurately stay at the preset position during a movement along the first direction, and so that the protrusion 41 already connected to the driving portion 2 may also accurately stay at the preset position.

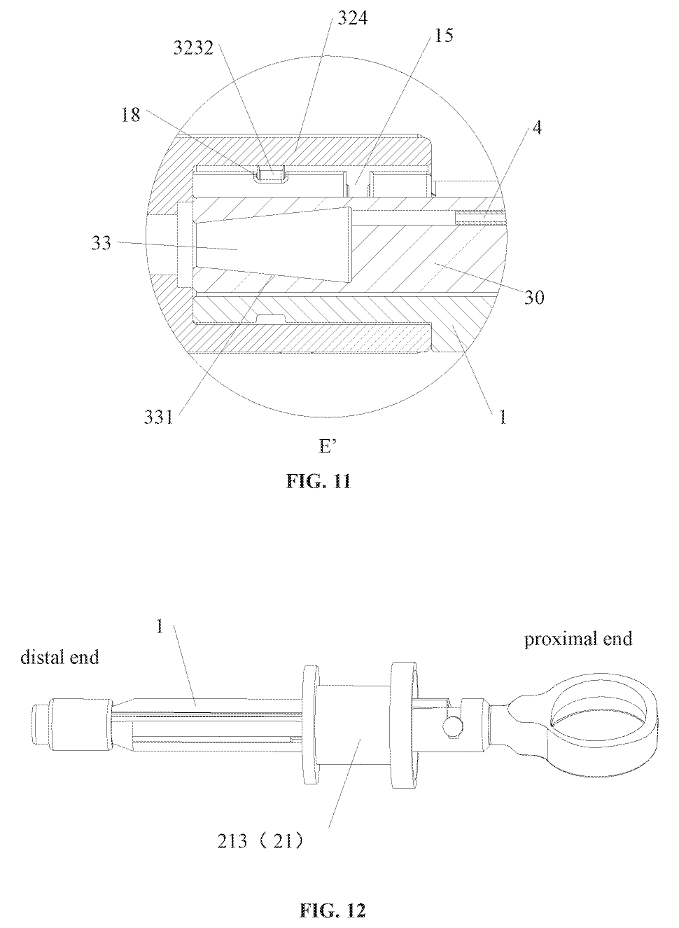

In some embodiments, the handle 100 further includes a first positioning assembly. The driving portion 2 and the protrusion 41 connected to the driving portion 2 may be positioned at the preset position through the first positioning assembly. When the driving portion 2 drives the transmission portions 4 to move from the distal end to the preset position, the first positioning assembly provides a first resistance that hinders the driving portion 2 from continuing to move toward the proximal end, to restrict the driving portion 2 and the protrusion 41 connected to the driving portion 2 at the preset position. It can be understood that, in some cases, when an external force acting on the driving portion 2 is large enough (for example, greater than the first resistance), the first positioning assembly may no longer restrict the driving portion 2 at the preset position, and the driving portion 2 will pass over the preset position and continue to move toward the proximal end. More descriptions regarding the first positioning assembly may be found in FIGS. 12-17A, 18-22, 29-34, and relevant descriptions thereof. In some embodiments, to avoid a state where the driving portion 2 slides idly and fails to find the preset position, the one or more second connection members 22 may always cooperate with the at least one first connection member, so that the second connection member 22 may smoothly stay at the preset position, thereby switching the required first connection member to form cooperation.

In some embodiments, referring to FIG. 2, the driving portion 2 may reach the preset position when driving the transmission portions 4 to move toward the proximal end until a pulling resistance is felt.

In some embodiments, when the driving portion 2 drives the transmission portions 4 to move from the distal end to the preset position, the transmission portions 4 control the moving components to perform a first operation. In some embodiments, when the driving portion 2 drives the transmission portions 4 to pass over the preset position and move toward the proximal end, the transmission portions 4 control the moving components to perform a second operation. The first operation is different from the second operation. For example, the first operation includes performing a surgical operation. When the driving portion 2 drives the transmission portions 4 to move from the distal end to the preset position, the transmission portions 4 control the moving components to perform the surgical operation such as clamping, electrocoagulation, cutting, sampling, or the like. The second operation includes disengagement of the moving components from the transmission portions 4. When the driving portion 2 drives the transmission portions 4 to pass over the preset position and move toward the proximal end, the moving components are controlled to disengage from the transmission portions 4.

In some embodiments, a release space 17 is reserved on the handheld portion 1 at a proximal end of the driving portion 2. The release space 17 may be in communication with the sliding guide groove 14, or may be a segment of the sliding guide groove 14 along the first direction. The release space 17 is located on a proximal side of the preset position, so that the driving portion 2 may drive the transmission portions 4 to move toward the proximal end of the handle 100 and slide into the release space 17, allowing the transmission portions 4 to disengage from the moving components connected to the transmission portions 4. It should be noted that, in an actual application scenario, if the moving components have no need to disengage from the transmission portions 4, the transmission portions 4 do not need to control the moving components to perform the second operation. Therefore, in some embodiments, the handheld portion 1 may not be provided with the release space 17.

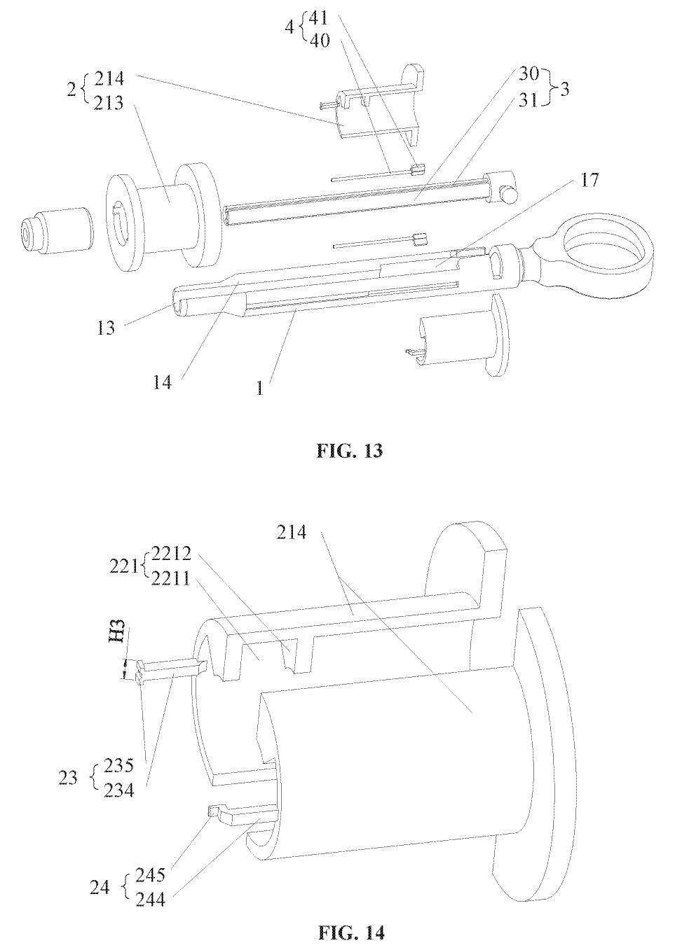

FIG. 12 is a schematic diagram illustrating a structure of a handle according to still some other embodiments of the present disclosure. FIG. 13 is an exploded view of a handle according to some embodiments of the present disclosure. FIG. 14 is a schematic diagram of a connection between an inner ring sleeve with a first positioning assembly and a second positioning assembly according to some embodiments of the present disclosure.

Differences between the handle 100 shown in FIGS. 12-13 and the handle 100 shown in FIGS. 2-4B include that the sliding member 21 includes a handheld ring sleeve 213 and an inner ring sleeve 214. The sliding member 21 is configured to drive the transmission portions 4 to operate. The first positioning assembly 23 is disposed between the sliding member 21 and the handheld portion 1, and is configured to position an opening end 2211 on the driving portion 2, so that the opening end 2211 is aligned with the protrusion 41 at the preset position. During a process in which the transmission portions 4 drive the moving components, the first positioning assembly 23 is used for positioning, so that the opening end 2211 of the groove 221 directly faces the protrusion 41.

In some embodiments, referring to FIGS. 12 and 13, the sliding member 21 includes the handheld ring sleeve 213 and the inner ring sleeve 214. The inner ring sleeve 214 is disposed inside the handheld ring sleeve 213. The handheld ring sleeve 213 is configured as an integrated structure. A proximal end and a distal end of the handheld ring sleeve 213 may be provided with a stopper or a retaining ring to form a groove for hand gripping. The inner ring sleeve 214 is configured as a split structure, so that the inner ring sleeve 214 may be detachably installed on the handheld portion 1. The groove 221 is provided on the inner ring sleeve 214. In some embodiments, the inner ring sleeve 214 is a split structure divided by a first plane. The first plane is a plane where an axis of the handle 100 is located. The groove 221 is formed by splicing two portions of the inner ring sleeve 214 located on both sides of the first plane.

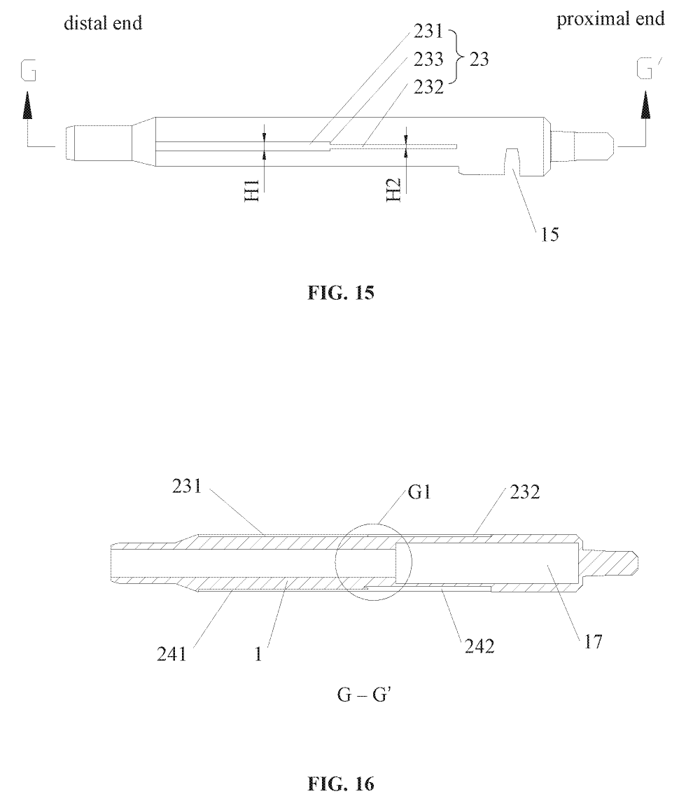

FIG. 15 is a schematic diagram illustrating a structure of a handheld portion according to still some other embodiments of the present disclosure.

In some embodiments, as shown in FIGS. 14 and 15, the first positioning assembly 23 includes a first sliding groove 231, a second sliding groove 232, a first limiting surface 233, a first positioning rod 234, and a first limiting block 235. The first sliding groove 231 and the second sliding groove 232 are formed on the handheld portion 1. The first sliding groove 231 is located at a distal end of the handheld portion 1. The second sliding groove 232 is located at a proximal end of the handheld portion 1. The first sliding groove 231 is in communication with the second sliding groove 232. A first limiting surface 233 facing the distal end is disposed between the first sliding groove 231 and the second sliding groove 232. The first positioning rod 234 is disposed on the sliding member 21 of the driving portion 2. The first positioning rod 234 may slide along the first sliding groove 231 and the second sliding groove 232. The first limiting block 235 is fixed at a distal end of the first positioning rod 234. The first limiting block 235 is slidably and cooperatively connected with the first sliding groove 231. When the first limiting block 235 moves toward the proximal end along the first sliding groove 231 and abuts against the first limiting surface 233, the first limiting surface 233 provides the first resistance that hinders the driving portion 2 from moving toward the proximal end, to position the driving portion 2 at the preset position.

In some embodiments, the first limiting surface 233 may be provided by a plate member or a block member disposed between the first sliding groove 231 and the second sliding groove 232. The plate member or the block member is provided with a channel for the first positioning rod 234 to pass through. A side surface of the plate member or the block member facing the distal end forms the first limiting surface 233. In some embodiments, the first limiting surface 233 may also be provided by a step surface arranged between the first sliding groove 231 and the second sliding groove 232. In some embodiments, a width H1 of the first sliding groove 231 is greater than a width H2 of the second sliding groove 232, to form the first limiting surface 233 facing the first sliding groove 231 at a connection position of the first sliding groove 231 and the second sliding groove 232. A width H3 of the first limiting block 235 is not greater than the width H1 of the first sliding groove 231. The width H3 of the first limiting block 235 is greater than the width H2 of the second sliding groove 232. Therefore, when the first limiting block 235 is fixed at the distal end of the first positioning rod 234, the first limiting block 235 may abut against the first limiting surface 233, so that the sliding member 21 (the handheld ring sleeve 213 and the inner ring sleeve 214) is located at the preset position, thereby positioning the groove 221. As a result, the opening end 2211 of the groove 221 may directly face the protrusion 41 when the first limiting block 235 abuts against the first limiting surface 233, facilitating different protrusions 41 to rotate into the groove 221 under the driving of the adjustment portion 3.

In some embodiments, the first limiting block 235 has a breaking state where the first limiting block 235 is squeezed against the first limiting surface 233 to disengage from the first positioning rod 234. When a driving force on the driving portion 2 is greater than the first resistance, the first limiting block 235 deforms, breaks, or falls off, and the driving portion 2 drives the transmission portions 4 to pass over the preset position and move toward the proximal end. Specifically, when the transmission portions 4 need to be disengaged from the moving components connected to the transmission portions 4, the sliding member 21 (the handheld ring sleeve 213 and the inner ring sleeve 214) and the transmission portions 4 connected to the sliding member 21 may be moved toward the proximal end of the handle into the release space 17. At this time, the first limiting block 235 is first squeezed against the first limiting surface 233 and receives a reaction force from the first limiting surface 233, and then breaks and disengages from the first positioning rod 234, so that the first limiting block 235 does not restrict the sliding member 21 (the handheld ring sleeve 213 and the inner ring sleeve 214) and the transmission portions 4 connected to the sliding member 21 from moving toward the proximal end of the handle into the release space 17.

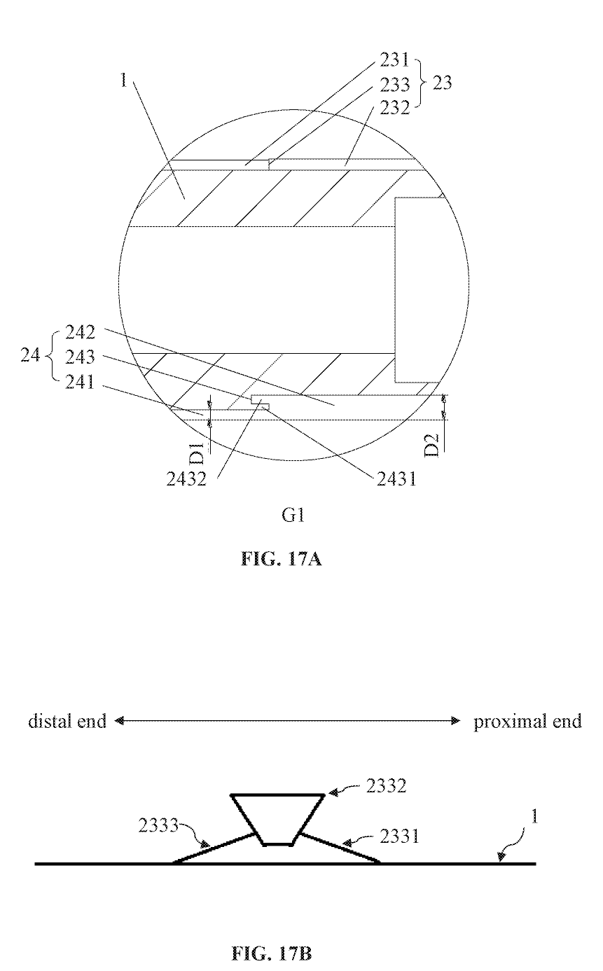

FIG. 17B is a schematic diagram of a first positioning assembly and a second positioning assembly according to still some other embodiments of the present disclosure.

In some embodiments, since the first limiting block 235 cannot be restored after deformation, breakage, or falling off, positioning cannot be achieved again through the first positioning assembly 23, which is not conducive to repeated use. Therefore, to make the first positioning assembly 23 reusable, in other embodiments, referring to FIG. 17B, the first positioning assembly 23 includes a first elastic piece 2331 and a first positioning block 2332. The first sliding groove and the second sliding groove may be communicating grooves with equal widths that communicate with each other (the communicating grooves are not shown in FIG. 17B). The first elastic piece 2331 is disposed on the handheld portion 1 and extends into the communication groove. The first positioning block 2332 is disposed on the driving portion (not shown in FIG. 17B). The first positioning block 2332 is slidable within the communication groove. In some embodiments, the first elastic piece 2331 includes a fixed end and a free end. The fixed end of the first elastic piece 2331 is connected to the handheld portion 1. The fixed end of the first elastic piece 2331 is located on a proximal end side of the preset position. The free end of the first elastic piece 2331 extends toward the distal end into the communication groove. In some embodiments, the first positioning block 2332 is disposed on the sliding member of the driving portion 2. The first positioning block 2332 includes a proximal end inclined surface. The proximal end inclined surface faces the proximal end of the handheld portion 1 and is inclined toward the fixed end of the first elastic piece 2331. In some embodiments, an angle formed between the proximal end inclined surface of the first positioning block 2332 and an inner side surface of the first elastic piece 2331 is an obtuse angle. The inner side surface of the first elastic piece 2331 refers to a surface of the first elastic piece 2331 facing the fixed end. When the driving portion 2 moves toward the proximal end, a resistance is relatively large when the proximal end inclined surface abuts against the free end of the first elastic piece 2331, to position the driving portion at the preset position. When the driving portion 2 needs to continue moving toward the proximal end beyond the preset position, the first positioning block 2332 may continue to be driven to press the free end of the first elastic piece 2331 toward the proximal end. The free end of the first elastic piece 2331 deflects outward from the communication groove along the proximal end inclined surface. After the free end of the first elastic piece 2331 moves out of the communication groove, the driving portion 2 is allowed to pass over the first elastic piece 2331 and move out of the preset position. In some embodiments, the first elastic piece 2331 is elastic. When an external force is removed, the first elastic piece 2331 may return to a natural state and re-enter the communication groove. When the driving portion moves toward the distal end to approach the preset position, the first positioning block 2332 (for example, a surface of the first positioning block 2332 facing the distal end) may press the first elastic piece 2331 to move outward from the communication groove, allowing the driving portion to pass over the first elastic piece 2331 and return to the preset position.

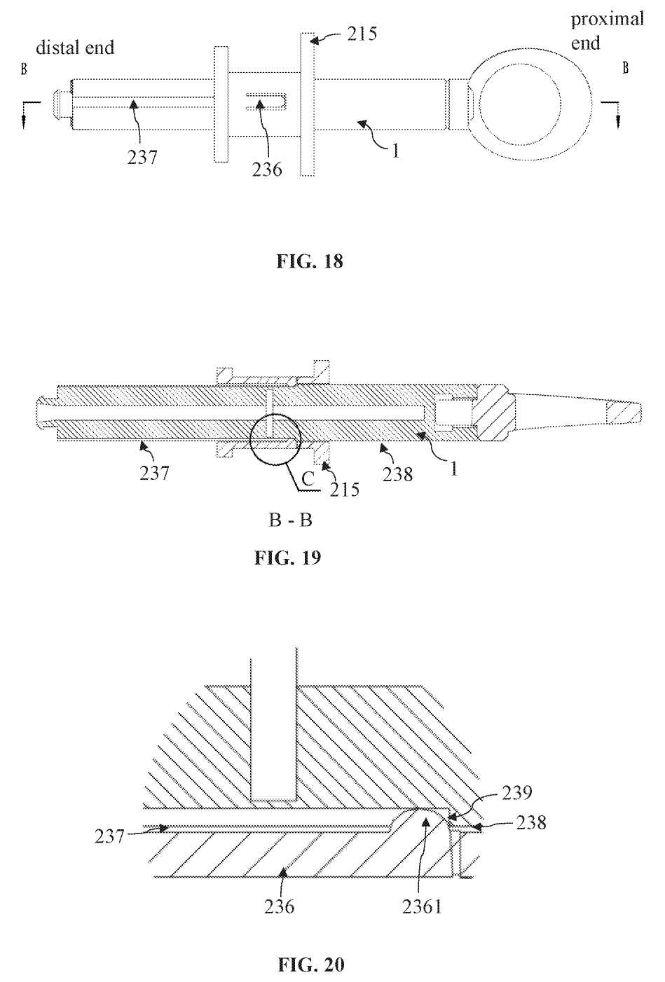

FIG. 18 is a schematic diagram illustrating a structure of a handheld portion and a driving portion according to still some other embodiments of the present disclosure.

Differences between the handheld portion and the driving portion shown in FIG. 18 and the handheld portion and the driving portion shown in FIGS. 12-13 include that the sliding member 21 includes a handheld sleeve 215. The handheld sleeve 215 is sleeved outside the handheld portion 1 and is configured to drive the transmission portions 4 to operate.

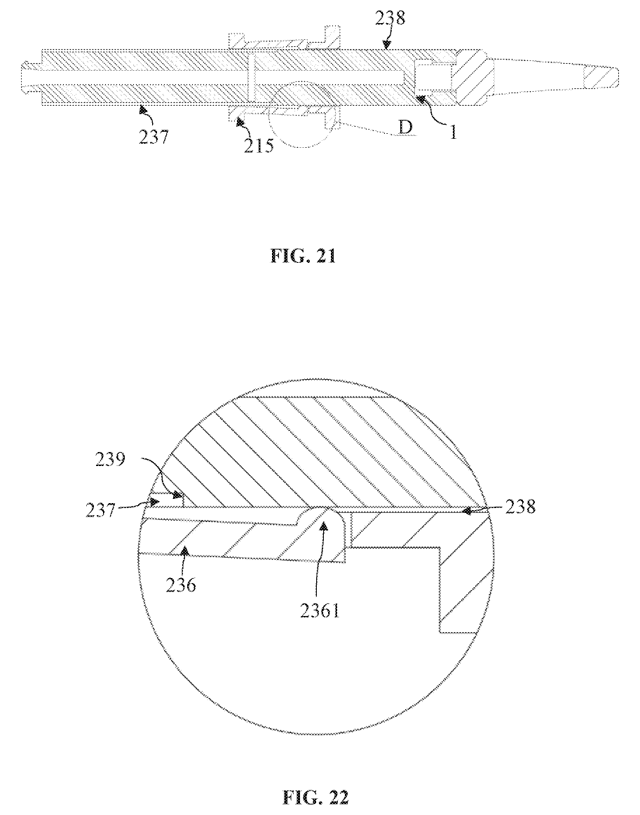

FIG. 19 is a schematic diagram of a cross-section taken along line B-B of a handheld portion and a driving portion when the driving portion is located at a preset position, according to some embodiments of the present disclosure. FIG. 20 is an enlarged view of a portion C in FIG. 19. FIG. 21 is a schematic diagram of a cross-section taken along line B-B of a handheld portion and a driving portion when the driving portion drives the transmission portions into a release space, according to some embodiments of the present disclosure. FIG. 22 is an enlarged view of a portion D in FIG. 21. FIGS. 19 and 20 illustrate an alternative embodiment of the first positioning assembly 23.

In some embodiments, as shown in FIGS. 18-20, the first positioning assembly 23 includes an elastic piece 236, a stopping portion 2361, a first sliding segment 237, a second sliding segment 238, and a limiting step 239. The elastic piece 236 is disposed on the handheld sleeve 215 of the driving portion 2. The elastic piece 236 includes a connection end connected to the handheld sleeve 215 and a free end separated from the handheld sleeve 215. The stopping portion 2361 is disposed at the free end of the elastic piece 236. The first sliding segment 237 and the second sliding segment 238 are groove structures disposed on the handheld portion 1 (in some embodiments, the first sliding segment 237 is a groove structure disposed on the handheld portion 1, and the second sliding segment 238 is an outer wall surface of the handheld portion 1 itself). The first sliding segment 237 is located at a distal end of the handheld portion 1. The second sliding segment 238 is located at a proximal end of the handheld portion 1. The first sliding segment 237 and the second sliding segment 238 communicate with each other. A limiting step 239 facing the distal end is disposed between the first sliding segment 237 and the second sliding segment 238. The stopping portion 2361 is slidably and cooperatively connected with the first sliding segment 237. When the stopping portion 2361 moves toward the proximal end along the first sliding segment 237 and abuts against the limiting step 239, the limiting step 239 provides a first resistance to hinder a movement of the driving portion 2 toward the proximal end, to position the driving portion 2 at the preset position.

In some embodiments, as shown in FIG. 18, the connection end of the elastic piece 236 may be located at a distal end of the elastic piece 236, and the free end of the elastic piece 236 may be located at a proximal end of the elastic piece 236. Alternatively, the connection end of the elastic piece 236 may be located at a proximal end of the elastic piece 236, and the free end of the elastic piece 236 may be located at a distal end of the elastic piece 236, which is not limited herein. In some embodiments, a radial dimension of the first sliding segment 237 (e.g., a groove depth) is greater than a radial dimension of the second sliding segment 238 (e.g., a groove depth or no groove structure), to form the limiting step 239 facing the distal end at a junction between the first sliding segment 237 and the second sliding segment 238. In some embodiments, the stopping portion 2361 is disposed to protrude in a radial direction of the handheld portion 1.

As shown in FIG. 20, when the handheld sleeve 215 is slid, and the stopping portion 2361 slides along the first sliding segment 237 to abut against the limiting step 239, the driving portion 2 reaches the preset position. At this time, the adjustment portion 3 may be operated to switch other protrusions 41 to cooperate with the groove 221, thereby driving different transmission portions 4 to connect to different moving components. In some embodiments, the elastic piece 236 is elastic. A driving force applied through the handheld sleeve 215 deforms the elastic piece 236 abutted against the limiting step 239 to generate an elastic force. When the elastic force increases to a certain extent, the stopping portion 2361 connected to the free end of the elastic piece 236 may pass over the limiting step 239 and continue sliding toward the proximal end along the second sliding segment 238. The elastic piece 236 returns to a natural state after the external force disappears, as shown in FIGS. 21 and 22. The handheld sleeve 215 and the transmission portions 4 connected to the handheld sleeve 215 may be moved toward the proximal end of the handle into the release space 17, to disconnect the transmission portions 4 from the moving components connected to the transmission portions 4. In some embodiments, a surface of the stopping portion 2361 contacting the limiting step 239 may be an arc surface, and/or the limiting step 239 may have an arc transition, so that the stopping portion 2361 may smoothly pass over the limiting step 239 under the action of the elastic force.

When the handheld sleeve 215 moves toward the distal end again, and the stopping portion 2361 slides toward the distal end along the second sliding segment 238, the stopping portion 2361 may smoothly cross the limiting step 239 under the driving force applied by the driving portion 2 to be repositioned at the preset position. The first positioning assembly 23 shown in FIGS. 18-22 does not experience irrecoverable situations such as breakage or falling off during processes such as positioning at the preset position and moving into the release space 17. The first positioning assembly 23 may be used repeatedly to achieve positioning.

FIG. 16 is a schematic diagram of a cross-section taken along line G-G' in FIG. 15. FIG. 17A is an enlarged view of a portion G1 in FIG. 16.

In some embodiments, as shown in FIGS. 16 and 17, the handle 100 further includes a second positioning assembly 24. When the driving portion 2 drives the transmission portions 4 to move from the proximal end to the preset position, the second positioning assembly 24 provides a second resistance to hinder the driving portion 2 from continuing to move toward the distal end. That is, the driving portion 2 drives the transmission portions 4 connected to the driving portion 2 to move toward the proximal end of the handle into the release space 17, and the transmission portions 4 may be disconnected from the connected moving components. When the driving portion 2 slides toward the distal end again, the second positioning assembly 26 may be used for positioning, so that the opening end 2211 of the groove 221 is at the preset position, to switch with a protrusion 41 that is not yet connected. It should be noted that embodiments of the present application may be provided with both the first positioning assembly 23 and the second positioning assembly 24. In some embodiments, only the first positioning assembly 23 or only the second positioning assembly 24 may be provided. In other embodiments, the first positioning assembly 23 and/or the second positioning assembly 24 may also be used in combination with the elastic reset member 6 shown in FIGS. 9-10, to make positioning more precise.

In some embodiments, the second positioning assembly 24 includes a third sliding groove 241, a fourth sliding groove 242, a second limiting surface 243, a second positioning rod 244, and a second limiting block 245. The third sliding groove 241 and the fourth sliding groove 242 are formed on the handheld portion 1. The third sliding groove 241 is located at a distal end of the handheld portion 1. The fourth sliding groove 242 is located at a proximal end of the handheld portion 1. The third sliding groove 241 communicates with the fourth sliding groove 242. A radial depth D1 of the third sliding groove 241 relative to the holding portion 1 is less than a radial depth D2 of the fourth sliding groove 242. At least a portion of the fourth sliding groove 242 is located on a proximal end side of the third sliding groove 241. A second limiting surface 243 is disposed at a proximal end of the third sliding groove 241, or a second limiting surface 243 is disposed at a distal end of the fourth sliding groove 242. Referring to FIG. 14, the second positioning rod 244 is disposed on the sliding member 21 of the driving portion 2. A proximal end of the second positioning rod 244 is connected to the driving portion 2. The second limiting block 245 is disposed at a distal end of the second positioning rod 244. The second positioning rod 244 is slidable along the third sliding groove 241 and the fourth sliding groove 242. The second limiting block 245 is slidably and cooperatively connected with the fourth sliding groove 242. When the second limiting block 245 moves toward the distal end along the fourth sliding groove 242 and abuts against the second limiting surface 243, the second limiting surface 243 provides a second resistance to hinder the movement of the driving portion 2 toward the distal end, to position the driving portion 2 at the preset position.

In some embodiments, due to a radial depth difference between the third sliding groove 241 and the fourth sliding groove 242, and at least a portion of the fourth sliding groove 242 being located on a proximal end side of the third sliding groove 241, a distal end of the fourth sliding groove 242 is connected to a proximal end of the third sliding groove 241. The second limiting surface 243 is formed on a proximal end surface of the third sliding groove 241 or a distal end surface of the fourth sliding groove 242. Taking the second limiting surface 243 formed on the distal end surface of the fourth sliding groove 242 as an example, in some embodiments, a stop flange 2431 extending toward the proximal end is formed at an outer side of the second limiting surface 243 close to the third sliding groove 241, to form a stopping groove 2432 between the second limiting surface 243 and the stop flange 2431. When the second limiting block 245 abuts against the second limiting surface 243, the second limiting block 245 may be embedded into the stopping groove 2432, restricting the driving portion 2 from sliding toward the distal end of the handheld portion 1. At this time, the sliding member 21 (the handheld ring sleeve 213 and the inner ring sleeve 214) is at the preset position, thereby positioning the groove 221. As a result, the opening end 2211 of the groove 221 may face the protrusion 41 directly when the second limiting block 245 abuts against the second limiting surface 243, facilitating different protrusions 41 to rotate into the groove 221 under the drive of the adjustment portion 3. Since the first positioning assembly 23 cannot accurately position the groove 221 after the first limiting block 235 breaks and disengages from the first positioning rod 234, the second limiting block 245 abutting against the second limiting surface 243 may be used for positioning at this time, to switch different protrusions 41 to cooperate with the groove 221, thereby driving different transmission portions 4 to disconnect from the moving components connected to the different transmission portions 4.

In some embodiments, the second positioning rod 244 is elastic. When the second limiting block 245 is in the fourth sliding groove 242, the second positioning rod 244 is in a natural state, i.e., an unstretched state. When the second limiting block 245 is in the third sliding groove 241, the second positioning rod 244 has an elastic restoring force to return to the natural state. When the second limiting block 245 moves from the distal end toward the proximal end and moves from the third sliding groove 241 to the fourth sliding groove 242, under an action of the elastic restoring force, the second positioning rod 244 drives the second limiting block 245 to be pulled back into the fourth sliding groove 242, so that the sliding member 21 may be repositioned to the preset position under a second resistance provided by the second limiting surface 243 to hinder the driving portion 2 from moving toward the distal end.

In some embodiments, the second limiting block 245 has a state of being broken off from the second positioning rod 244 by squeezing the second limiting surface 243. Specifically, when a driving force applied to the driving portion 2 is greater than the second resistance, the second limiting block 245 deforms, breaks, or falls off. The driving portion 2 drives the transmission portions 4 to pass over the preset position toward the distal end.

Similar to the first positioning assembly 23, in some embodiments, after the second limiting block 245 deforms, breaks, or falls off, the second limiting block 245 cannot be restored. The second limiting block 245 can no longer achieve positioning through the second positioning assembly 24, which is not conducive to repeated use. Therefore, to enable the second positioning assembly 24 to be reused, in other embodiments, referring to FIG. 17B, the second positioning assembly 24 includes a second elastic piece (e.g., the second elastic piece 2333) and a second positioning block. In some embodiments, the second positioning block and the first positioning block 2332 may be the same positioning block. The second elastic piece is disposed on the handheld portion 1 and extends into the communication groove. The second positioning block is disposed on the driving portion 2. The second positioning block is slidable within the communication groove. In some embodiments, the second elastic piece includes a fixed end and a free end. The fixed end of the second elastic piece is connected to the handheld portion 1. The fixed end of the second elastic piece is located on a distal end of the preset position. The free end of the second elastic piece extends toward the proximal end into the communication groove. In some embodiments, the second positioning block includes a distal inclined surface. The distal inclined surface faces the distal end of the handheld portion 1 and is inclined toward the fixed end of the second elastic piece. In some embodiments, an angle formed between the distal inclined surface of the second positioning block and an inner side surface of the second elastic piece is an obtuse angle. The inner side surface of the second elastic piece refers to a surface of the second elastic piece facing the fixed end. When the driving portion 2 moves toward the proximal end, the second positioning block (e.g., a surface of the second positioning block facing the distal end) may squeeze the second elastic piece to move out of the communication groove, allowing the driving portion 2 to pass over the second elastic piece and reach the preset position. In some embodiments, the second elastic piece is elastic. When the distal inclined surface abuts against the free end of the second elastic piece, the second elastic piece may return to a natural state after removal of an external force. When the driving portion 2 moves from a proximal side of the preset position toward the distal end, after the distal inclined surface abuts against the free end of the second elastic piece, a resistance is relatively large, thereby positioning the driving portion 2 at the preset position. Since the distal inclined surface of the second positioning block faces the distal end and is inclined toward the fixed end of the second elastic piece, the free end of the second elastic piece deflects outward from the communication groove along the distal inclined surface. After the free end of the second elastic piece moves out of the communication groove, the driving portion 2 is allowed to pass over the second elastic piece and move out of the preset position.

It should be noted that, in some embodiments, the first positioning assembly 23 and the second positioning assembly 24 may be provided simultaneously. In this case, as shown in FIG. 17B, the first positioning assembly 23 and the second positioning assembly 24 share a positioning block (the first positioning block or the second positioning block). The first elastic piece and the second elastic piece are respectively disposed on a proximal side and a distal side of the preset position. A proximal inclined surface and the distal inclined surface are respectively disposed on a side surface of the positioning block facing the proximal end and a side surface of the positioning block facing the distal end. When the positioning block is located between the first elastic piece and the second elastic piece, the driving portion 2 is located at the preset position.

In other embodiments, the second positioning assembly 24 may also have a structure similar to the first positioning assembly 23 shown in FIG. 18. Specifically, the second positioning assembly 24 includes a fourth elastic piece, a second stopping portion, a third sliding segment, a fourth sliding segment, and a second limiting step (the elastic piece 236 in the first positioning assembly 23 may also be referred to as a third elastic piece, the stopping portion 2361 may also be referred to as a first stopping portion, and the limiting step 239 may also be referred to as a first limiting step). The fourth elastic piece is disposed on the handheld sleeve of the driving portion 2. The fourth elastic piece includes a connection end connected to the handheld sleeve and a free end separated from the handheld sleeve. The second stopping portion is disposed at the free end of the fourth elastic piece. The third sliding segment and the fourth sliding segment are disposed on the handheld portion 1. The third sliding segment is located at a proximal end of the handheld portion 1. The fourth sliding segment is located at a distal end of the handheld portion 1. The third sliding segment and the fourth sliding segment communicate with each other. A second limiting step facing the proximal end is disposed between the third sliding segment and the fourth sliding segment. The second stopping portion is slidably and cooperatively connected with the third sliding segment. When the second stopping portion moves along the third sliding segment toward the distal end and abuts against the second limiting step, the second limiting step provides a second resistance to the second stopping portion that hinders the driving portion 2 from moving toward the distal end, thereby positioning the driving portion 2 at the preset position. An external force applied to the driving portion 2 may be further increased to make the second stopping portion pass over the second limiting step. The driving portion 2 may continue to move toward the distal end. It should be noted that, in this embodiment, the second positioning assembly 24 may be provided simultaneously with the first positioning assembly 23 in any of the aforementioned embodiments. Taking the second positioning assembly 24 of this embodiment being provided simultaneously with the first positioning assembly shown in FIG. 18 as an example, the third elastic piece and the fourth elastic piece may be simultaneously disposed on the driving portion 2. For example, the third elastic piece and the fourth elastic piece may be alternately distributed along a circumferential direction of the driving portion 2. The third sliding segment and the first sliding segment may be the same sliding segment. The first limiting step and the second limiting step are respectively located at two ends of the third sliding segment/first sliding segment (i.e., the first limiting step is located at the proximal end, and the second limiting step is located at the distal end).

FIG. 29 is an exploded view of a handle according to some embodiments of the present disclosure. FIG. 29 illustrates another embodiment of the first positioning assembly 23.

Differences between the handle 100 shown in FIG. 29 and the handle 100 shown in FIGS. 23-28 include that the first positioning assembly 23 includes a blocking portion 2310. The blocking portion 2310 is rotatable relative to the handheld portion 1 around the first direction. The blocking portion 2310 is switchable between a blocking position and a release position by rotation. When the blocking portion 2310 is in the blocking position, the driving portion 2 slides along the first direction to the preset position to contact the blocking portion 2310. When the blocking portion 2310 is in the release position, a movement of the driving portion 2 is unrestricted by the blocking portion 2310.

In some embodiments, the blocking portion 2310 is disposed to protrude at least partially from an outer surface of the adjustment portion 3. The blocking portion 2310 is disposed to protrude radially outward toward the adjustment portion 3, enabling the blocking portion 2310 to be switched onto a sliding path of the driving portion 2 (i.e., the blocking position), thereby blocking the driving portion 2 (the handheld sleeve 215) to position the driving portion 2 at the preset position. A radial direction of the adjustment portion 3 refers to a direction from a center axis of the adjustment portion 3 as a reference toward the outer surface (outer periphery).

In some implementations, the blocking portion 2310 may be controlled to rotate by an independently provided rotation control mechanism. The blocking portion 2310 is connected to the rotation control mechanism. The rotation control mechanism is operated to control the blocking portion 2310 to rotate independently of the adjustment portion 3. In some embodiments, for ease of operation by an operator, the blocking portion 2310 is disposed on the adjustment portion 3. The blocking portion 2310 is connected to the support rod 30 of the adjustment portion 3. Rotation of the rotary cap 324 may drive the support rod 30 to rotate. The blocking portion 2310 rotates with the rotation of the support rod 30 within the handheld portion 1 to switch between the blocking position and the release position.

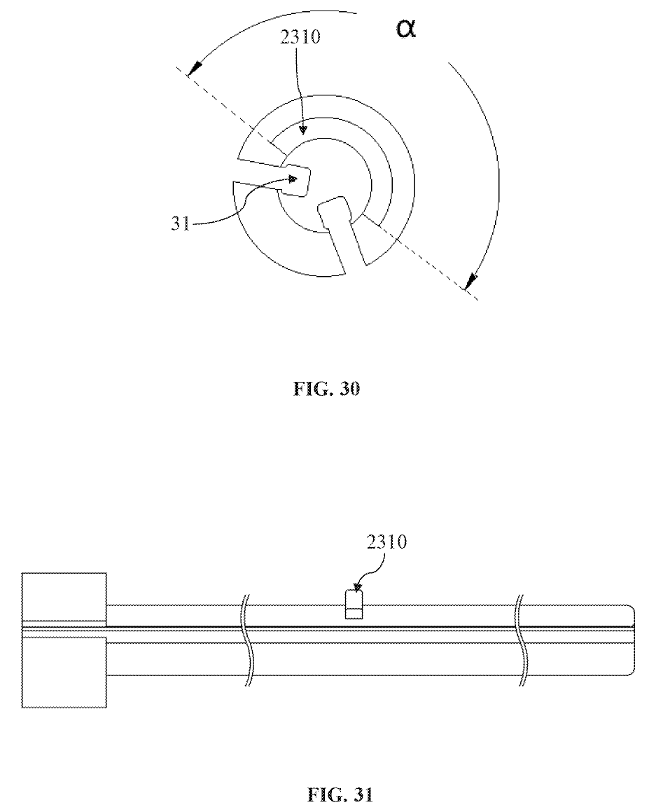

FIG. 30 is a cross-sectional view of a support rod provided with a blocking portion according to some embodiments of the present disclosure. FIG. 31 is a schematic diagram illustrating a structure of a support rod according to some embodiments of the present disclosure.

In some embodiments, as shown in FIGS. 30 and 31, the blocking portion 2310 is disposed to protrude from an outer surface of the support rod 30. Specifically, the blocking portion 2310 is a fan-shaped protrusion disposed around the rotation axis of the support rod 30. The blocking portion 2310 extends radially outward toward the support rod 30 to form a block on the sliding path of the driving portion 2. In some embodiments, to cooperate with the blocking portion 2310, a limiting portion 2311 is disposed on an inner side surface of the handheld sleeve 215 of the driving portion 2 close to the support rod 30. The limiting portion 2311 extends and protrudes toward the support rod 30. In some embodiments, the limiting portion 2311 may be a proximal outer wall of the groove 221.

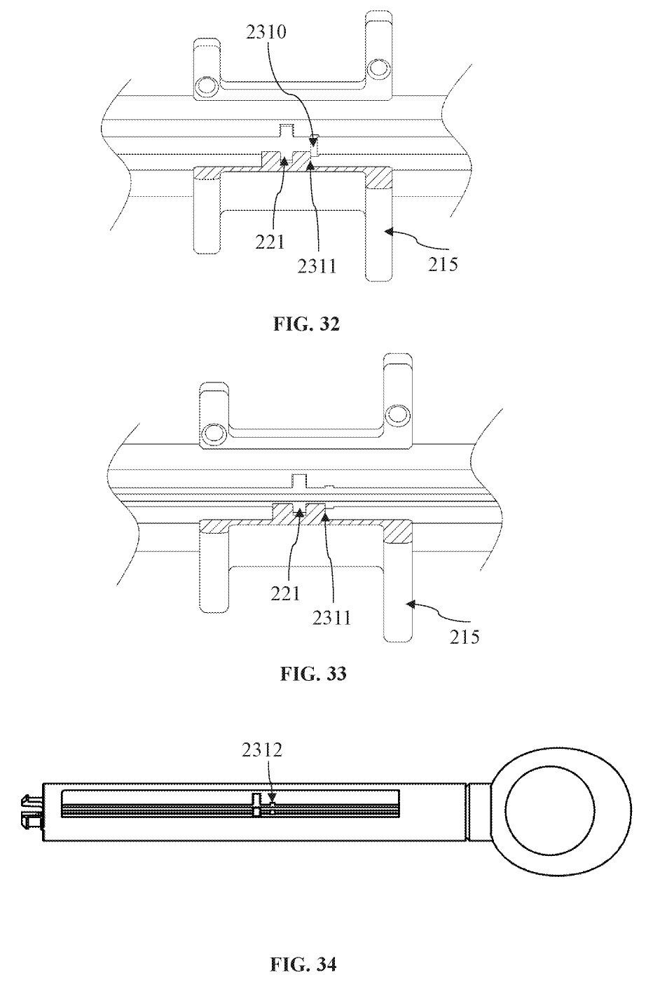

FIG. 32 is a schematic diagram illustrating a structure of a limiting portion abutting against a blocking portion according to some embodiments of the present disclosure. FIG. 33 is a schematic diagram illustrating a structure of a blocking portion located at a release position according to some embodiments of the present disclosure.

As shown in FIG. 32, the blocking portion 2310 rotates out from the mounting cavity 13 of the handheld portion 1 toward the sliding guide groove 14. At this time, the blocking portion 2310 is in the blocking position. The handheld sleeve 215 is pulled toward the proximal end. When the limiting portion 2311 of the handheld sleeve 215 abuts against the blocking portion 2310, the handheld sleeve 215 reaches the preset position. The transmission portions 4 connected to the driving portion 2 may be switched as required. In some embodiments, the groove 221 cooperatively connected with the transmission portions 4 is disposed on the limiting portion 2311. The groove 221 extends through the limiting portion 2311 around the rotation axis of the support rod 30, facilitating adjustment of the first connection members of the transmission portions 4 to rotate into the groove 221 for cooperative connection with the driving portion 2.

As shown in FIG. 33, when the handheld sleeve 215 needs to be moved to the release space, the blocking portion 2310 is rotated again, causing the blocking portion 2310 to rotate into the mounting cavity 13 of the handheld portion 1. At this time, the blocking portion 2310 is in the release position. The limiting portion 2311 is no longer abutted against by the blocking portion 2310. The handheld sleeve 215 may continue to slide toward the proximal end and reach the release space.

FIG. 34 is a schematic diagram illustrating a structure of a handheld portion according to some embodiments of the present disclosure.

In some embodiments, to enable the blocking portion 2310 to extend as far as possible radially outward toward the support rod 30, a radial dimension of the blocking portion 2310 should be as large as possible. To accommodate the radial dimension of the blocking portion 2310, a receiving cavity 2312 is provided within the mounting cavity 13 of the handheld portion 1. The receiving cavity 2312 is a groove extending radially around the support rod 30. When the blocking portion 2310 rotates into the receiving cavity 2312 of the handheld portion 1, the blocking portion 2310 is in the release position. In some embodiments, an angle α of the fan-shaped protrusion forming the blocking portion 2310 is less than or equal to an angle of the receiving cavity 2312 along a circumferential direction. In some embodiments, the angle α of the fan-shaped protrusion may range from 170° to 180°. In some embodiments, the angle α of the fan-shaped protrusion may range from 175° to 180°. In some embodiments, the angle α of the fan-shaped protrusion may range from 177° to 179°. In some embodiments, the angle α of the fan-shaped protrusion may be 178°.

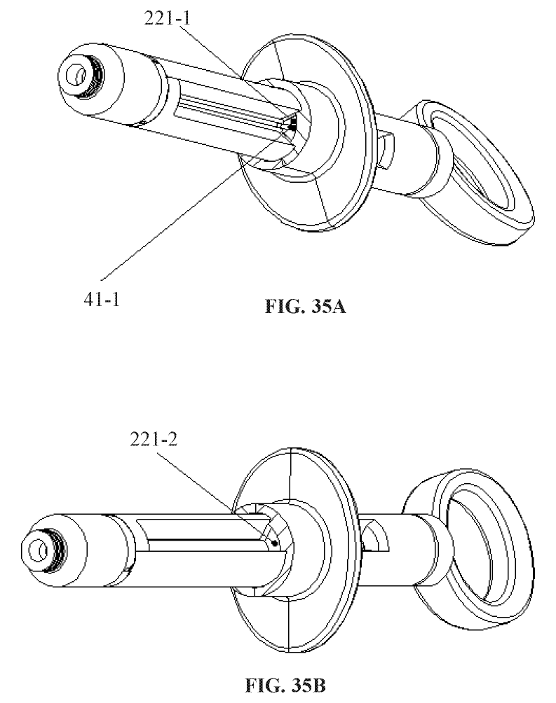

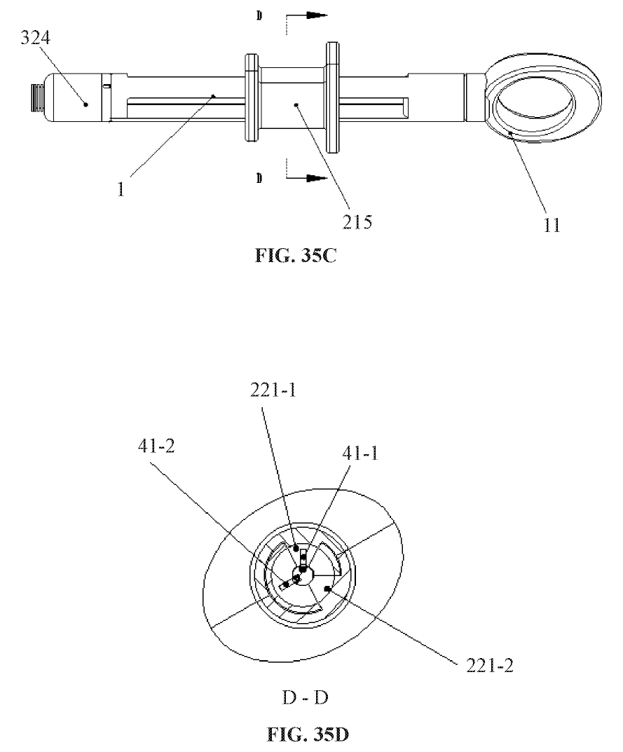

FIGS. 35A-37D are schematic diagrams each illustrating a gear switching process of a handle according to some embodiments of the present disclosure. The gear switching process refers to a process of switching connections between different transmission members 40 and the driving portion 2 or disconnecting the different transmission members 40 from the driving portion 2.

In some embodiments, referring to FIGS. 35A-35D, the handheld sleeve 215 includes two grooves (a groove 221-1 and a groove 221-2). The handle 100 includes two transmission members. Each of the two transmission members includes a first connection member (a first connection member 41-1 and a first connection member 41-2). The handle 100 is in a first gear. At this time, the first connection member (e.g., the first connection member 41-1) on one of the two transmission members is located in the groove 221-1. At this time, the handheld sleeve 215 is pushed or pulled to cause the transmission member (e.g., the first connection member 41-1) to move accordingly. When gear switching is required, the handheld sleeve 215 is pulled toward the proximal end until a limiting portion 2311 abuts against a blocking portion 2310. At this time, the handheld sleeve 215 reaches a preset position, and gear switching may be performed.

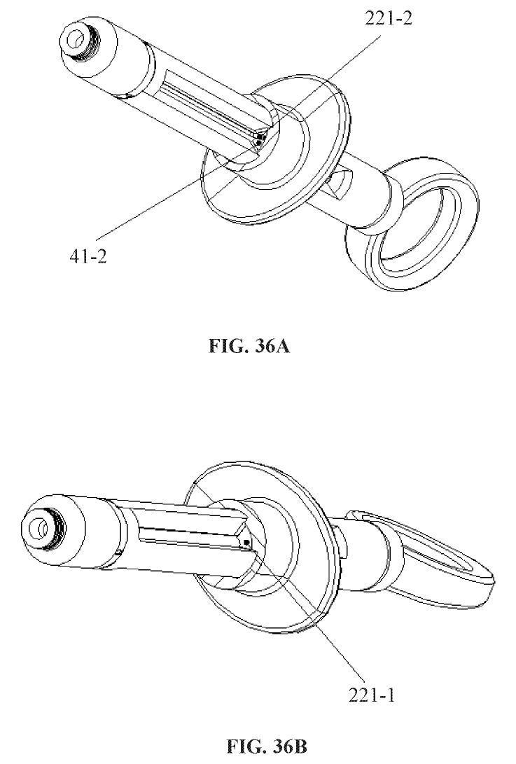

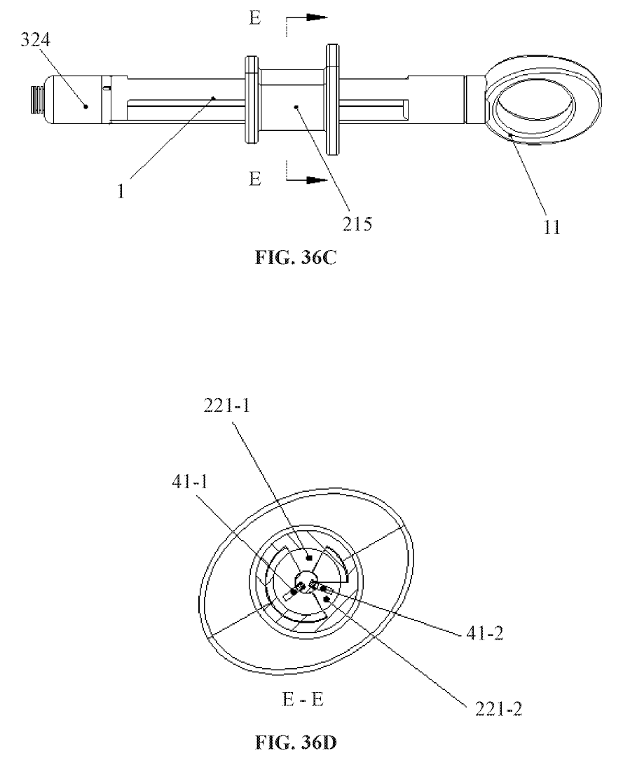

In some embodiments, referring to FIGS. 36A-36D, when the handle 100 needs to be switched to a second gear, the rotary cap 324 is rotated. The first connection member 41-1 enters the handheld portion 1. The first connection member 41-2 on another transmission member enters the groove 221-2. Similar to the handle 100 being in the first gear, the handle 100 in the second gear may also drive a connected transmission member to move by pushing or pulling the handheld sleeve 215.

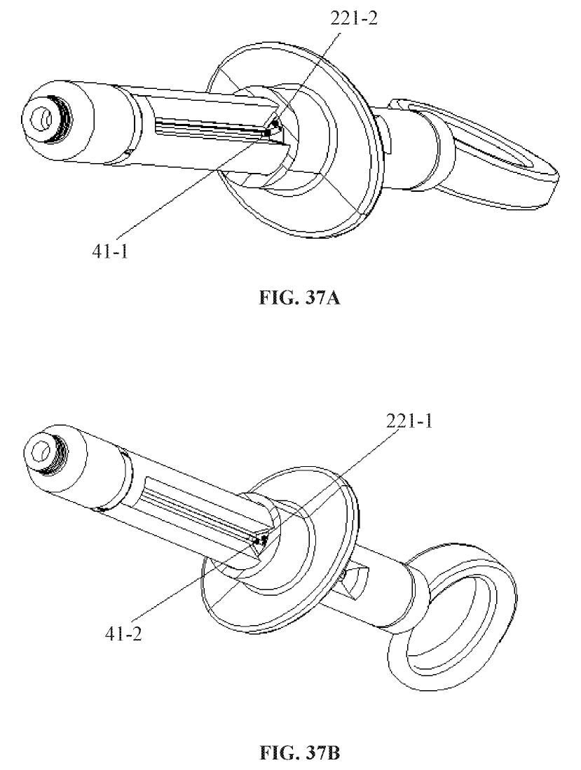

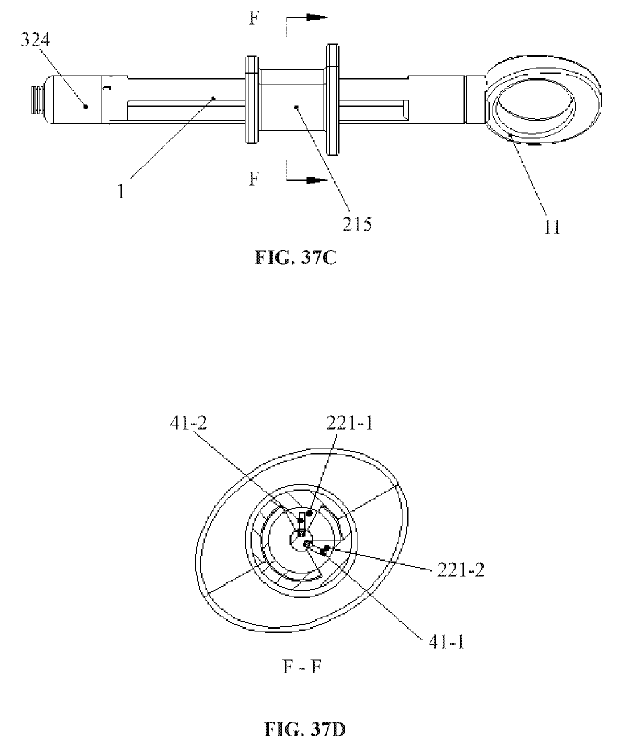

In some embodiments, referring to FIGS. 37A-37D, when a moving component connected to a transmission member needs to be released, the rotary cap 324 may be rotated again to switch the handle 100 to a third gear. At this time, the blocking portion 2310 enters the handheld portion 1. The first connection member 41-1 is located in the groove 221-2. The first connection member 41-2 is located in the groove 221-1. Pulling the handheld sleeve 215 backward may achieve synchronous release of a plurality of moving components.

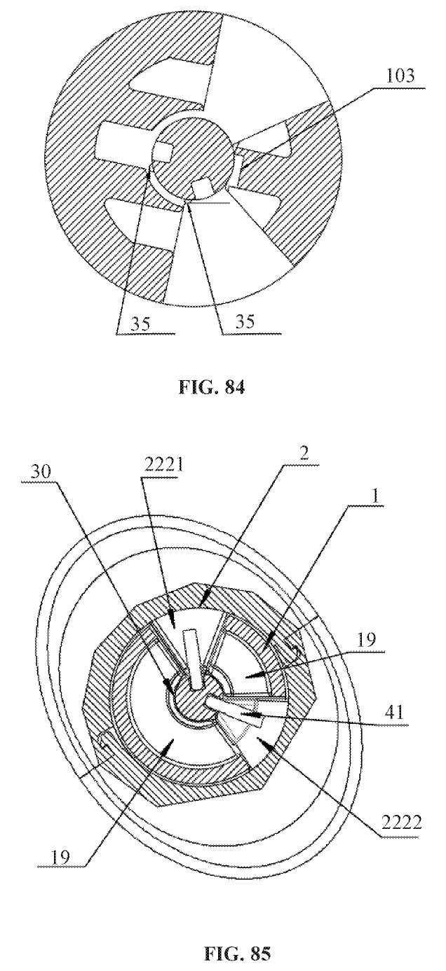

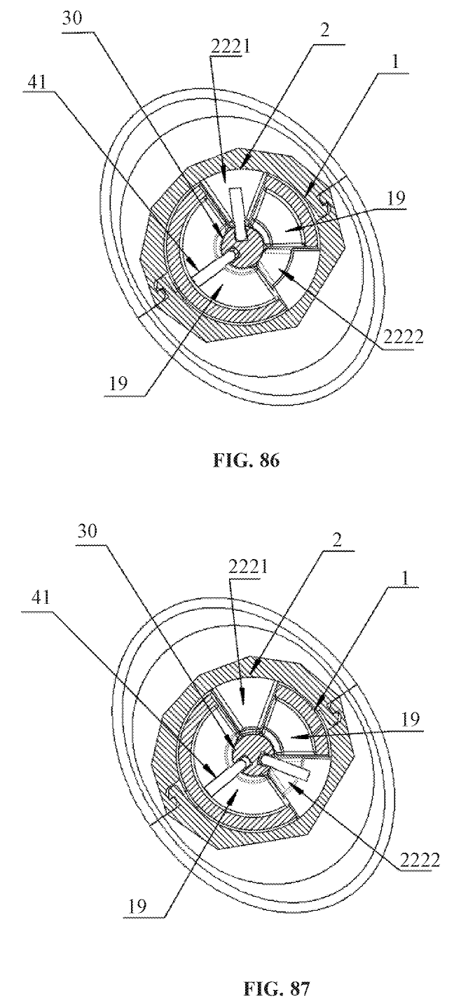

FIG. 83 is a schematic diagram illustrating a structure of a handle in a second gear according to some embodiments of the present disclosure. FIG. 84 is a schematic diagram illustrating a structure of a handle in a first gear according to some embodiments of the present disclosure. FIG. 85 is a schematic diagram illustrating a structure of a handle in a third gear according to some other embodiments of the present disclosure. FIG. 86 is a schematic diagram illustrating a structure of a handle in a second gear according to some other embodiments of the present disclosure. FIG. 87 is a schematic diagram illustrating a structure of a handle in a first gear according to some other embodiments of the present disclosure.

In some embodiments, as shown in FIGS. 76 and 85, when the rotary cap 324 is in a third gear, the support protrusion 35 on the support rod 30 is at both ends of an avoidance groove 103, the first connector 411 is located in the first groove portion 2221, and the second connector 412 is located in the second groove portion 2222.

In some embodiments, as shown in FIGS. 83 and 86, when the rotary cap 324 rotates in a direction around an axial direction, the support protrusion 35 on the support rod 30 abuts against a support wall 102, the first connector 411 enters one of the axial limiting spaces 19, and the second connector 412 enters the first groove portion 2221. At this point, the rotary cap 324 is in a second gear.

In some embodiments, as shown in FIGS. 84 and 87, when the rotary cap 324 continues to rotate in the direction around the axial direction, the support protrusion 35 on the support rod 30 abuts against the support wall 102, the first connector 411 enters the second groove portion 2222, and the second connector 412 enters one of the axial limiting spaces 19. At this point, the rotary cap 324 is in a first gear.

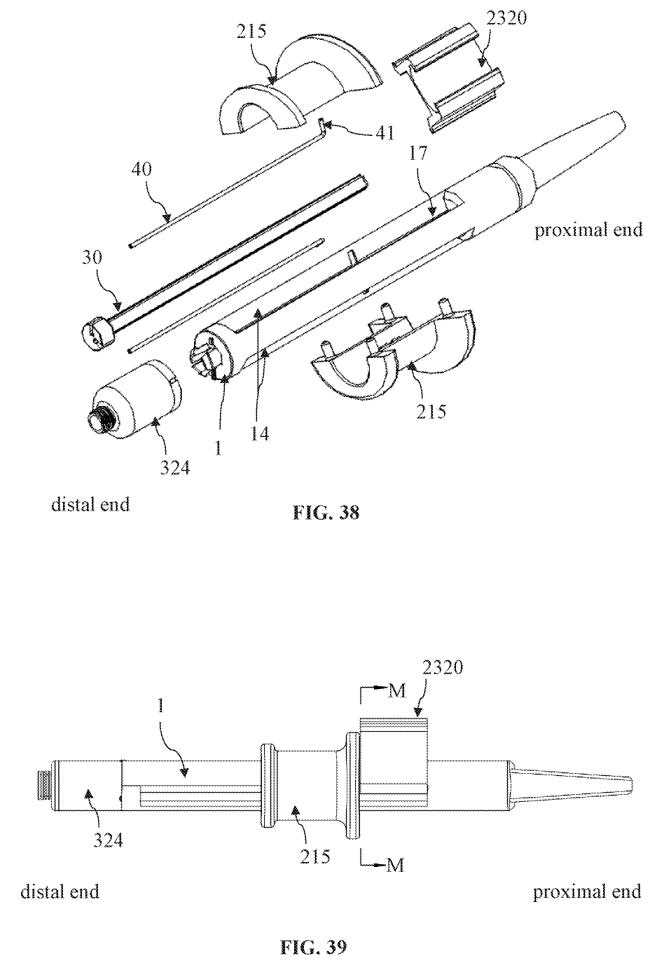

FIGS. 38-42B illustrate another embodiment of the first positioning assembly 23.

Differences between the handle 100 shown in FIG. 38 and the handle 100 shown in FIGS. 23-28 include that the first positioning assembly 23 includes a locking member 2320. The locking member 2320 is detachably connected to the handheld portion 1.

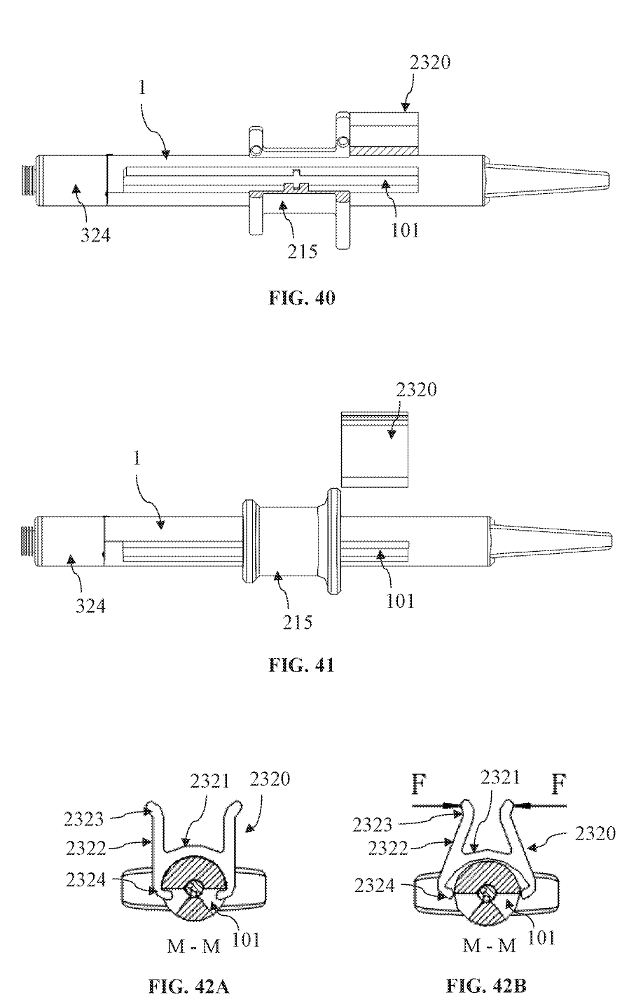

As shown in FIGS. 39 and 40, when the locking member 2320 is connected to a preset connection position of the handheld portion 1, the driving portion 2 slides along a first direction (e.g., slides from the distal end toward the proximal end) to the preset position and contacts the locking member 2320. The locking member 2320 positions the driving portion 2 at the preset position. In some embodiments, the handheld portion 1 is provided with a locking groove 101 engaged with the locking member 2320. In some embodiments, a dimension of the locking groove 101 along the first direction may be the same as or similar to a dimension of the locking member 2320 along the first direction. Arranging the locking groove 101 at the preset connection position may allow the locking member 2320 to be directly connected to the preset connection position of the handheld portion 1 when the locking member 2320 is engaged with the locking groove 101. In some embodiments, the dimension of the locking groove 101 along the first direction may be greater than the dimension of the locking member 2320 along the first direction. After the locking member 2320 is engaged with the locking groove 101, the locking member 2320 may slide along the locking groove 101. When the locking member 2320 slides to a proximal end of the locking groove 101, the locking member 2320 is connected to the preset connection position of the handheld portion 1. In some embodiments, the locking groove 101 is a sliding guide groove 14. A limiting portion is provided at a proximal end of the sliding guide groove 14. When the locking member 2320 slides to abut against the restriction portion, the locking member 2320 is connected to the preset connection position of the handheld portion 1.

As shown in FIG. 41, when a connection between the locking member 2320 and the handheld portion 1 is released, a movement of the driving portion 2 is unrestricted by the locking member 2320. Therefore, an operator may quickly remove movement restriction on the driving portion 2 by removing the locking member 2320, which features simple operation and easy use.

As shown in FIGS. 42A and 42B, the locking member 2320 includes a locking portion 2324 and a detachment portion 2323. The locking portion 2324 has a clamping state and an open state. When the locking portion 2324 is in the clamping state, the locking member 2320 may be engaged in the locking groove 101. When the locking portion 2324 is in the open state, the locking member 2320 may be removed from the locking groove 101. When the detachment portion 2323 is subjected to a force, the detachment portion 2323 may drive the locking portion 2324 to move, thereby causing the locking portion 2324 to switch between the clamping state and the open state.

In some embodiments, the locking member 2320 may include two oppositely arranged locking arms 2322 and an elastic connecting arm 2321 connecting the two locking arms 2322. The elastic connecting arm 2321 may be connected to middle sections of the two locking arms 2322. A portion of each of the two locking arms 2322 located on one side of the elastic connecting arm 2321 (e.g., an end portion) constitutes the locking portion 2324. A portion of each of the two locking arms 2322 located on another side of the elastic connecting arm 2321 (e.g., an end portion) constitutes the detachment portion 2323. With such an arrangement, the locking portion 2324 and the detachment portion 2323 form a lever structure with a connection point between the elastic connecting arm 2321 and each of the two locking arms 2322 as a fulcrum. The locking portion 2324 may be controlled to enter the open state when a disassembly force F is applied to the detachment portion 2323 to cause a relative movement of the detachment portion 2323.

It is understandable that the structure of the locking member 2320 shown in FIGS. 42A and 42B does not constitute a limitation on the locking member 2320. In some alternative embodiments, the locking member 2320 may also be designed as a clip structure. A clip head of the clip structure may constitute the locking portion. A clip tail of the clip structure may constitute the detachment portion. In some embodiments, the clip head and the clip tail of the clip structure are hinged through a pivot shaft. An elastic body such as a torsion spring is arranged at the clip tail to maintain the clamping state.

FIG. 61 is a schematic diagram illustrating a structure of a handle according to some further embodiments of the present disclosure.

In some embodiments, as shown in FIG. 61, the handle 100 includes a proximal finger ring 11, the handheld portion 1, the driving portion 2, the support rod 30, the first connection member 41, and the locking member 2320.

In some embodiments, the locking member 2320 is configured for limiting during gear shifting. The driving portion 2 is snapped onto the handheld portion 1 and can slide relative to the handheld portion 1 along an axial direction. The first connection member slides within the sliding groove 31 of the support rod 30.

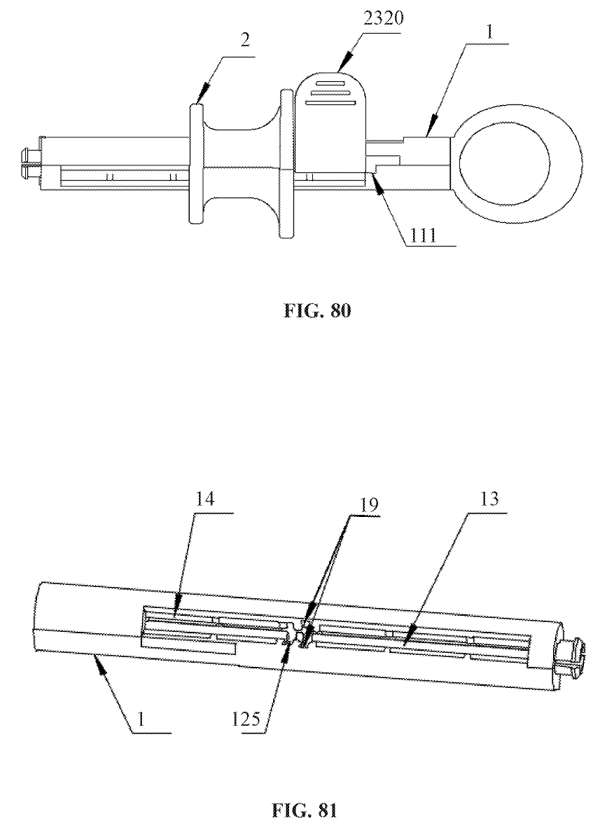

FIG. 80 is a schematic diagram illustrating a structure of a handle according to some further embodiments of the present disclosure.

In some embodiments, as shown in FIG. 80, the handle 100 includes the handheld portion 1, the locking member 2320, and the driving portion 2. A proximal end of the sliding groove 31 is provided with a clearance groove 111 communicating with the sliding groove 31.

In some embodiments, when the locking member 2320 is connected at a preset connection position of the handheld portion 1, the driving portion 2 slides along a first direction (e.g., slides from a distal end toward the proximal end) and drives the locking member 2320 to move toward the proximal end of the handle. The locking member 2320 enters the clearance groove 111. When a connection between the locking member 2320 and the handheld portion 1 is released, movement of the driving portion 2 is not restricted by the locking member 2320.

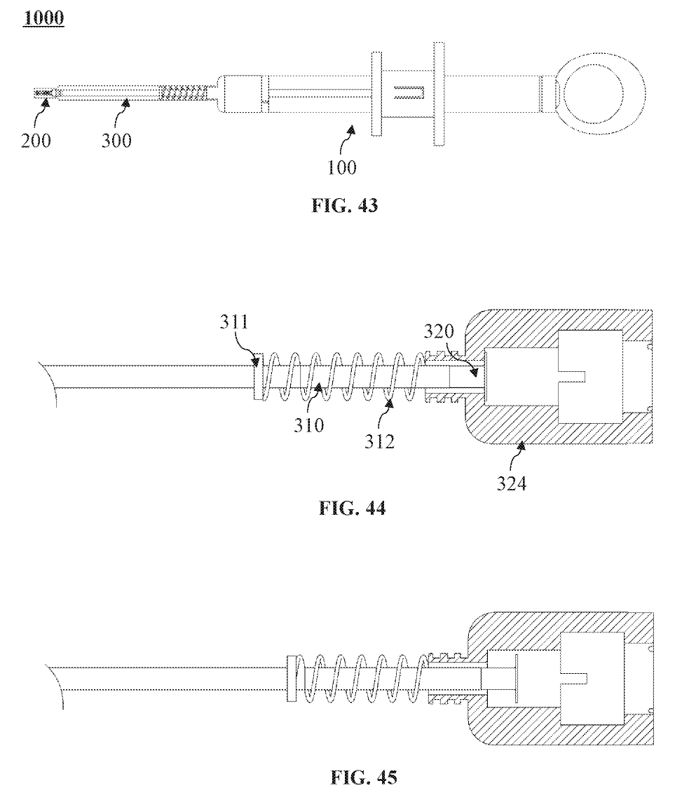

FIG. 43 is a schematic diagram illustrating a structure of a medical device according to some embodiments of the present disclosure.

As shown in FIG. 43, a medical device 1000 includes a handle 100 according to any of the aforementioned embodiments. The medical device 1000 further includes a plurality of moving components 200. The plurality of moving components 200 are connected one-to-one with a plurality of transmission portions 4 of the handle 100. An adjustment portion 3 of the handle 100 drives different transmission portions 4 among the plurality of transmission portions 4 to connect to a driving portion 2 of the handle 100, so as to drive different moving components among the plurality of moving components 200 to operate. In some embodiments, the medical device 1000 includes a delivery assembly 300. A proximal end of the delivery assembly 300 is connected to the handle 100. The plurality of moving components 200 are disposed at a distal end of the delivery assembly 300. Each of the plurality of moving components 200 is operatively coupled to at least one of the plurality of transmission portions 4. The delivery assembly 300 is configured to connect and pull the plurality of moving components 200. The delivery assembly 300 may also be configured to provide substances (e.g., gas, liquid, etc.) required for a medical procedure or a medical operation.

In some embodiments, the handle 100 includes the adjustment portion 3. The adjustment portion 3 includes a rotary cap 324 (referring to FIGS. 23-28) and a support rod 30 that are connected to each other. The rotary cap 324 is rotatably connected to a distal end of the handheld portion 1. To simplify a structure of the medical device 1000, the rotary cap 324 may be extended to form a connection port for connecting to the delivery assembly 300.

FIG. 44 is a schematic diagram illustrating a structure of a delivery assembly according to some embodiments of the present disclosure. FIG. 45 is a schematic diagram illustrating a structure of a movement change of the delivery assembly in FIG. 44.

In some embodiments, as shown in FIGS. 44 and 45, the delivery assembly 300 includes a sheath tube 310 and a sleeve tube 320. A traction cable is disposed inside the sheath tube 310. The sleeve tube 320 is disposed at a proximal end of the sheath tube 310. The sheath tube 310 is connected through cooperation between the sleeve tube 320 fixed on the sheath tube 310 and the rotary cap 324. In some embodiments, the rotary cap 324 is a hollow structure with a distal opening. A proximal end of the sheath tube 310 extends into the distal opening of the rotary cap 324 through the sleeve tube 320. The sleeve tube 320 is disposed inside the rotary cap 324. A flanged structure is disposed at a proximal end of the sleeve tube 320. The flange structure may prevent the sleeve tube 320 from disengaging from the distal opening of the rotary cap 324.

When the medical device 1000 enters a natural body lumen along with an endoscope, the medical device 1000 also bends along with the endoscope due to a presence of different curved segments. When the delivery assembly 300 bends, a pull wire inside the delivery assembly 300 also bends. Since the pull wire is a columnar structure with a certain thickness, when the pull wire bends, a compression side with a small bending radius and a tension side pointed by the bending radius from the compression side appear on the pull wire. The pull wire will lean toward the tension side inside the sheath tube 310. Since an end of the pull wire connected to the moving components 200 is fixed, the pull wire near the handle 100 side will retract into the sheath tube 310 accordingly. This phenomenon is referred to as "lost motion". To compensate for the "lost motion", i.e., a length by which the pull wire retracts into the sheath tube 310, in some embodiments, the sheath tube 310 further includes a limiting ring 311 and an elastic member 312. The limiting ring 311 is disposed outside the rotary cap 324. The elastic member 312 is disposed between the limiting ring 311 and the rotary cap 324. In some embodiments, the limiting ring 311 is fixed on an outer wall surface of the sheath tube 310. The elastic member 312 is sleeved on the sheath tube 310. A distal end of the elastic member 312 is connected to or contacts the limiting ring 311. A proximal end of the elastic member 312 is connected to or contacts the rotary cap 324. The rotary cap 324 internally reserves a reserved space for displacement of the sheath tube 310 and the sleeve tube 320 toward the proximal end. As shown in FIG. 44, when the medical device 1000 experiences the "lost motion" due to entering the human body along with the endoscope, because the medical device 1000 includes the pull wire and under a non-release gear (where a transmission portion connected to the driving portion is not in a release space), the pull wire is always fixed inside the handheld portion 1. As shown in FIG. 45, the elastic member 312 is compressed by a compression force generated by the lost motion. At this time, the sheath tube 310 moves into the reserved space inside the rotary cap 324 driven by the compressive force. A displacement of the sheath tube 310 is a compression amount of the elastic member 312. This is equivalent to the sheath tube 310 shortening its own length under the action of the limiting ring 311 and the elastic member 312, thereby compensating for the lost motion.

FIG. 62 is a schematic diagram illustrating a structure of a delivery assembly according to some other embodiments of the present disclosure.

In some embodiments, as shown in FIGS. 62 and 63, the delivery assembly 300 includes the rotary cap 324, a silicone cap 330, a lubrication tube 340, a steel wire hook 350, the limiting ring 311, the elastic member 312, a transition connection tube 360, a metal connection member 370, a plastic-coated spring hose 380, a spring end head 390, and an elastic pin 3110. In some embodiments, the elastic member 312 is a spring, or the like.

The silicone cap 330 refers to a component that prevents external impurities from entering an internal structure while allowing the rotary cap to rotate inside.

The lubricating tube 340 refers to a tubular component configured to reduce friction between the steel wire hook 350 and a tube wall at a bending portion.

The steel wire hook 350 refers to a component configured to transmit a force of a rotary cap.

The transition connecting tube 360 refers to an adapter tube configured to connect tubes of different diameters or materials.

The metal connection member 370 refers to a component configured to fix a moving component to the rotary cap.

The plastic-coated spring hose 380 refers to a flexible tube configured to protect an internal steel wire.

The spring end 390 refers to a connector configured to prevent delamination or fraying of an end portion of the hose and to provide an interface for connection with other components.

The elastic pin 3110 refers to a component configured to fix a relative position between two parts (e.g., fixing the steel wire hook and a tightening tube).

In some embodiments, the spring end 390 is fixedly connected to the plastic-coated spring hose 380, and the metal connection member 370 is fixedly connected to the plastic-coated spring hose 380. The aforementioned fixed connection may be achieved through various manners such as injection molding, crimping, or the like.

In some embodiments, a first connection member and a proximal end of the steel wire hook 350 are welded or directly integrally formed. The driving portion 2 drives the first connection member to move along an axial direction. The first connection member further drives the steel wire hook 350 to move. The rotary cap 324 is connected to an exterior of the support rod 30 via a key connection or a snap-fit connection. The rotary cap 324 rotates to different gears and simultaneously drives the support rod 30 to rotate synchronously.

The rotary cap is provided with three gears during rotation (a first gear, a second gear, and a third gear. When at the third gear, synchronous opening and closing of dual clamping arms may be achieved. When at the second gear, opening and closing of only one side clamping arm may be achieved. When at the first gear, opening and closing of only the other side clamping arm may be achieved).

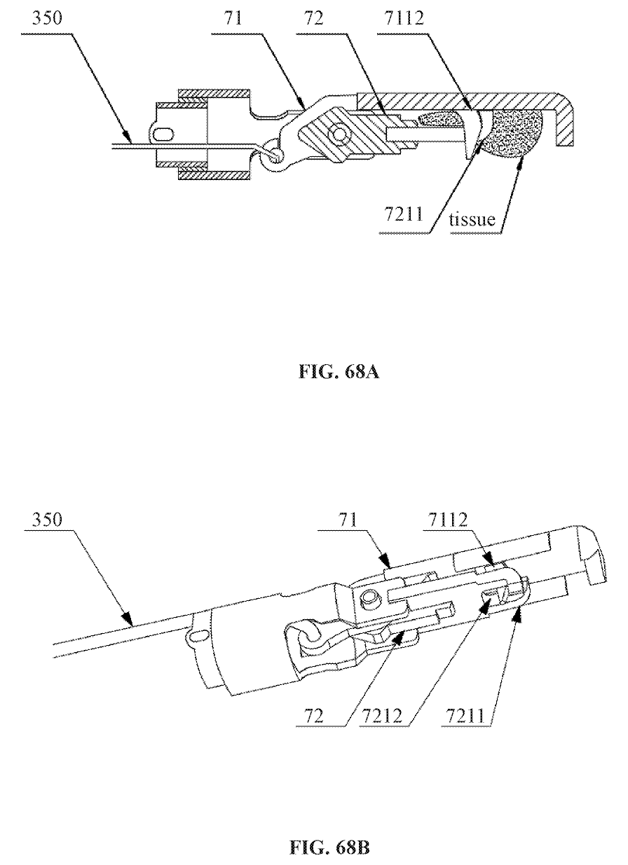

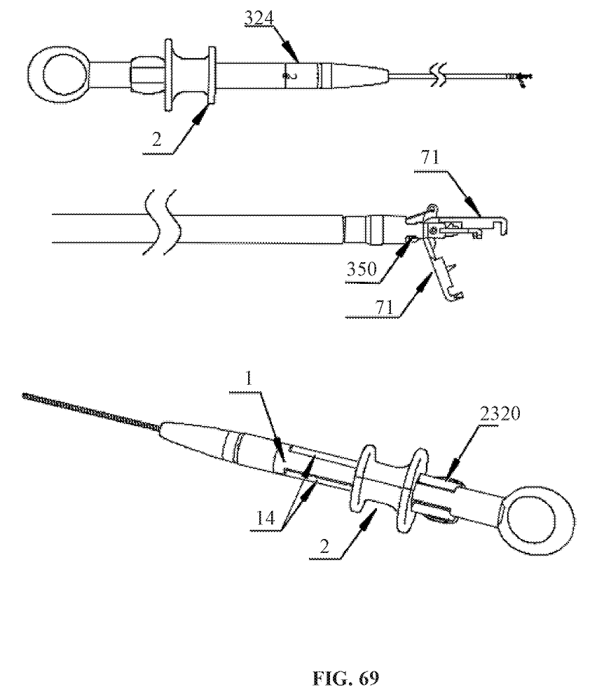

FIG. 67 is a schematic structural diagram illustrating a closed state of a clamping jaw and a middle piece according to some embodiments of the present disclosure. FIGS. 68A and 68B are schematic structural diagrams illustrating use of a clamping portion according to some embodiments of the present disclosure.

As shown in FIG. 67, when the medical device 1000 passes through an endoscopic instrument channel, a side wing wrapping structure 7111 wraps an intermediate limiting piece 721 and a protrusion structure 7112 inside the clamping jaw 71, thereby preventing the instrument channel from being scratched.

As shown in FIG. 68A, after the clamping portion passes through the instrument channel, the rotary cap 324 is rotated to switch to a second gear. The driving portion 2 is pushed and pulled. Since the first connection member and a tail end of the steel wire hook 350 are welded, axial movement of the first connection member drives the steel wire hook 350 to move synchronously. A distal end of the steel wire hook 350 is hooked on the tail hook 7121 of the clamping jaw 71. Therefore, the steel wire hook 350 drives one side clamping jaw 71 to open and close. When clamping a wound surface, the protrusion structure 7112 may pierce into tissue. When the one side clamping jaw 71 reaches a closed position, the clamping jaw 71 and the intermediate piece 72 clamp the tissue. Simultaneously, the protrusion structure 7112 pierces into the tissue and passes through a tooth gap 7212 between two distal teeth 7211, thereby making unilateral clamping more secure. As shown in FIG. 68B, after unilateral clamping of tissue is completed, the one side clamping jaw 71 is in a closed state. The protrusion structure 7112 is located inside the teeth 7211 and passes through the tooth gap 7212.

A position of the protrusion structure 7112 is set at a middle portion of the clamping jaw. After wound surface clamping is completed, the tissue fixed between the clamping jaw 71 and the intermediate piece 72 has a tendency to expand outward. A main force-bearing position is at the protrusion structure 7112. A design of a clamping head portion is similar to a lever principle. When the barb position is subjected to force, a shorter force arm results in a smaller force on a portion of the tail hook 7121 locked inside the tightening tube 74. A gap for opening the clamping jaw becomes smaller, and locking of the tissue becomes more secure.

As shown in FIG. 69, the rotary cap 324 is rotated to switch to the first gear. The one side clamping jaw 71 and the intermediate piece 72 that have clamped the tissue complete pre-clamping. The tissue is pulled to another side of the wound surface. The driving portion 2 is pushed. The steel wire hook 350 moves axially toward a clamping head end. A clamping jaw 71 on a side that has not clamped tissue opens. The driving portion 2 is pulled. The steel wire hook 350 moves axially toward a handle end. The other side clamping jaw 71 closes to complete wound surface clamping.

In some embodiments, a use process of the clamping portion is as follows:

In S1, the clamping portion is inserted along an endoscopic channel in a closed state until a first target position is reached. At this time, the driving portion 2 is located at a preset position (at this time, a first connector 411 and a second connector 412 are located in a first groove portion 2221 and a second groove portion 2222, respectively).

In S2, the rotary cap 324 is rotated to switch to the second gear (or the first gear). At this time, the first connector 411 moves from the first groove portion 2221 into an axial limiting space 19 of the handheld portion 1. The second connector 412 moves from the second groove portion 2222 into the first groove portion 2221. The driving portion 2 is pushed toward the distal end. The first connector 411 located in the first groove portion 2221 follows the driving portion 2 to move toward the distal end, thereby opening a corresponding side clamping arm. The driving portion 2 is then pulled toward the proximal end. The second connector 412 located in the first groove portion 2221 follows the driving portion 2 to move toward the proximal end, thereby closing the corresponding side clamping arm to achieve clamping of first tissue. During clamping, the protrusion structure 7112 on the clamping arm pierces into the tissue.

In S3, the clamping arm closed in S2 is pulled along with the endoscope to a second target position.

In S4, after S2 is completed, the driving portion 2 has returned to the preset position. After S3 is completed, the rotary cap 324 is further rotated to switch to the first gear (or the second gear). At this time, the second connector 412 located in the first groove portion 2221 moves into the axial limiting space 19 of the handheld portion 1. The first connector 411 located in the first groove portion 2221 moves into the second groove portion 2222. The driving portion 2 is pushed toward the distal end. The first connector 411 located in the second groove portion 2222 follows the driving portion 2 to move, thereby opening a corresponding side clamping arm (i.e., a side clamping arm different from that in S2). The driving portion 2 is then pulled toward the proximal end. The first connector 411 located in the second groove portion 2222 follows the driving portion 2 to move toward the proximal end, thereby closing the corresponding side clamping arm to achieve clamping of second tissue. During clamping, the protrusion structure 7112 on the clamping arm pierces into the tissue.

In S5, after S2 is completed, the driving portion 2 returns to the preset position. The rotary cap 324 is rotated to switch to the third gear. At this time, the first connector 411 and the second connector 412 move into the first groove portion 2221 and the second groove portion 2222, respectively. The locking member 2320 is removed. The driving portion 2 is pulled backward continuously. At this time, the driving portion 2 may drive both the first connector 411 and the second connector 412 to move toward the proximal end. The driving portion 2 drives the steel wire hook 350 to pull and deform tail hooks 7121 of clamping jaw connecting pieces. The tail hooks 7121 are locked inside the tightening tube 74, thereby completing a release and separation process. After S2 is completed, the driving portion 2 returns to the preset position, and after the rotary cap 324 is rotated to switch to the third gear, the driving portion 2 may also be directly pulled to drive the locking member 2320 to move toward the proximal end of the handle. The locking member 2320 enters the clearance groove 111, or the positioning tab is deformed or broken, so as to complete the release and separation process.

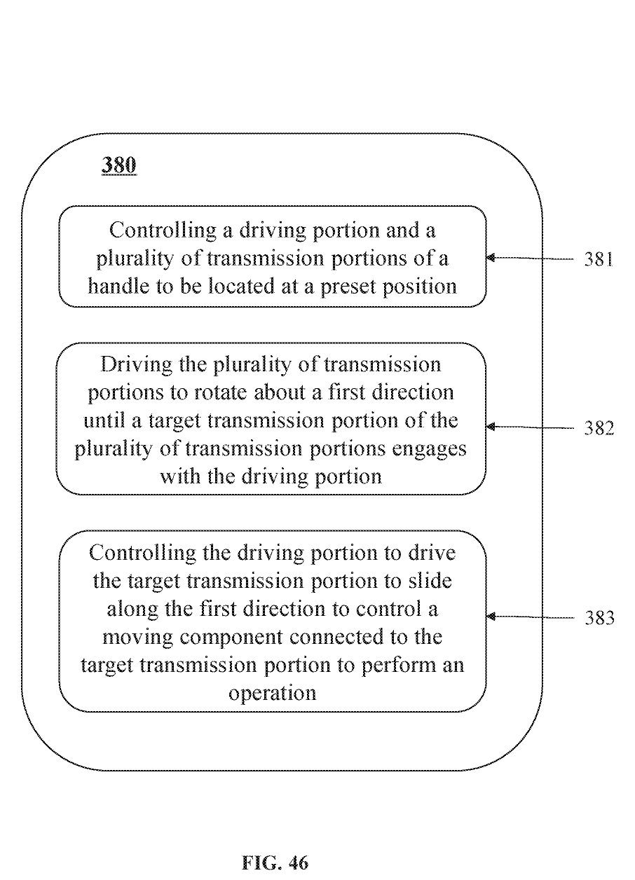

FIG. 46 is a flowchart illustrating an exemplary process of a method for operating a medical device according to some embodiments of the present disclosure.

As shown in FIG. 46, an embodiment of the present disclosure provides a method 380 for operating a medical device. The method 380 is applied to the medical device 1000 shown in FIG. 43. The medical device 1000 includes a handle 100, a delivery assembly 300, and a plurality of moving components 200. The handle 100 includes a handheld portion, a driving portion, and a plurality of transmission portions configured to be operatively coupled to a plurality of moving components at a distal end of the medical device. The handheld portion is slidably engaged with the driving portion. The driving portion is switchably connected to at least one of the plurality of transmission portions, and the driving portion is switchable between being connected to only one of the plurality of transmission portions and being simultaneously connected to at least two of the plurality of transmission portions. A proximal end of the delivery assembly is connected to the handle. The plurality of moving components are disposed at a distal end of the delivery assembly. Each of the plurality of moving components is drivingly connected to at least one of the plurality of transmission portions. The method 380 includes following steps.

In 381, a driving portion and a plurality of transmission portions of a handle may be controlled to be located at a preset position.

In some embodiments, a driving portion of a handle 100 slides along a first direction. The driving portion is positioned at the preset position by a first positioning assembly (e.g., the first positioning assembly 23). In some embodiments, referring to FIGS. 14 and 15, the driving portion 2 slides along the first direction, driving a first limiting block 235 to move toward the proximal end along a first sliding groove 231. When the first limiting block 235 abuts against a first limiting surface 233, the first limiting surface 233 provides a first resistance hindering the driving portion 2 from moving toward the proximal end, so as to position the driving portion 2 at the preset position. In some embodiments, referring to FIGS. 18-20, the driving portion 2 slides along the first direction, driving a stopping portion 2361 to move toward the proximal end along a first sliding segment 237. When the stopping portion 2361 abuts against a limiting step 239, the limiting step 239 provides the first resistance hindering the driving portion 2 from moving toward the proximal end for the stopping portion 2361, so as to position the driving portion 2 at the preset position. In some embodiments, referring to FIG. 29, the driving portion 2 is pulled toward the proximal end. When a limiting portion 2311 of the driving portion 2 abuts against a blocking portion 2310, the driving portion 2 reaches the preset position. In other embodiments, referring to FIGS. 9 and 10, the driving portion 2 may be caused to reach a preset position by using an elastic reset member 6. Specifically, an external force applied to the driving portion 2 is removed, so that the driving portion 2 and the transmission portions 4 engaged with the driving portion 2 return to the preset position under the restoring force of the elastic reset member 6. In some embodiments, referring to FIG. 2, the driving portion 2 drives the transmission portions 4 to displace toward the proximal end until the driving portion 2 reaches the preset position when a pulling resistance is felt. In some embodiments, as shown in FIGS. 39 and 40, when the locking member 2320 is connected to a preset connection position of the handheld portion 1, the driving portion 2 slides along the first direction (e.g., slides from the distal end toward the proximal end) to the preset position to contact the locking member 2320, and the locking member 2320 positions the driving portion 2 at the preset position.

In 382, the plurality of transmission portions may be driven to rotate about a first direction until a target transmission portion of the plurality of transmission portions engages with the driving portion.

In some embodiments, the adjustment portion is rotated to drive the plurality of transmission portions to rotate around the first direction. In some embodiments, referring to FIGS. 6 and 23, by rotating the rotary cap 324, the support rod 30 is rotated, and the transmission portion 4 disposed on the support rod 30 is rotated accordingly. In some embodiments, the support rod 30 is rotated until the first connection member disposed on the target transmission portion cooperates with the second connection member 22 on the driving portion 2. In some embodiments, the support rod 30 is rotated until a protrusion 41 disposed on the target transmission portion snaps into a groove 221 on the driving portion 2.

In 383, the driving portion may be controlled to drive the target transmission portion to slide along the first direction to control a moving component connected to the target transmission portion to perform an operation.

In some embodiments, the driving portion is driven toward the distal end or the proximal end of the handle, so that the driving portion drives the target transmission portion to slide synchronously along the first direction, to drive the moving component connected to the target transmission portion to perform an operation (e.g., opening/closing or sliding).

In some embodiments, the driving portion is controlled to move from the distal end toward the preset position to drive the moving components to perform a first operation. In some embodiments, the preset position may be an initial position of the driving portion. During use of the medical device, the driving portion may be located at the distal end of the handle. Controlling the driving portion to return from the distal end to the preset position allows the driving portion to drive the transmission portions to control the moving components to perform the first operation, such as a diagnosis and treatment operation (e.g., clamping, electrocoagulation, cutting, etc.). In some embodiments, after the movement of the moving components is completed, the driving portion drives the transmission portions to slide synchronously to the preset position. Referring to step 381, the driving portion and the plurality of transmission portions are positioned at the preset position.

In some embodiments, after the driving portion and the plurality of transmission portions are repositioned at the preset position, step 382 and step 383 may be repeated, or the driving portion may be controlled to move past the preset position toward the proximal end to drive the moving components to perform a second operation.

In some embodiments, the driving portion is used to drive the transmission portions to slide to the release space, so that the driving portion drives the moving components to perform the second operation. For example, the moving components disengage from the connected transmission portions. In some embodiments, referring to FIGS. 13-15, a driving force greater than a first resistance is applied to the driving portion 2, causing the first limiting block 235 to deform, break, or fall off. The driving portion 2 drives the transmission portions 4 to move past the preset position toward the proximal end to the release space 17. In some embodiments, referring to FIGS. 20-22, a driving force is applied to the driving portion 2, causing the elastic piece 236 abutting against the limiting step 239 to deform and generate an elastic force. When the elastic force increases to a certain extent, the stopping portion 2361 connected to the free end of the elastic piece 236 may pass over the limiting step 239 and continue to slide along the second sliding segment 238 toward the proximal end. In some embodiments, referring to FIGS. 32-33, the blocking portion 2310 is rotated so that the blocking portion 2310 is located at a release position. A limiting portion 2311 is no longer abutted against by the blocking portion 2310, and the driving portion 2 may be driven to continue sliding toward the proximal end to reach the release space (e.g., the release space 17). In some embodiments, referring to FIGS. 41-42B, by applying a disassembly force F to the detachment portion 2323 of the locking member 2320 to cause a relative movement of the detachment portion 2323, the locking portion 2324 of the locking member 2320 is controlled to enter an open state. The locking member 2320 may be removed from the handheld portion 1. The movement of the driving portion 2 is unrestricted by the locking member 2320 and may continue sliding toward the proximal end to reach the release space (e.g., the release space 17).

The above embodiments are merely preferred embodiments of the present invention and are not intended to limit the scope of protection of the present invention. Any non-substantial changes and substitutions made by those skilled in the art based on the present invention fall within the scope of protection claimed by the present invention.

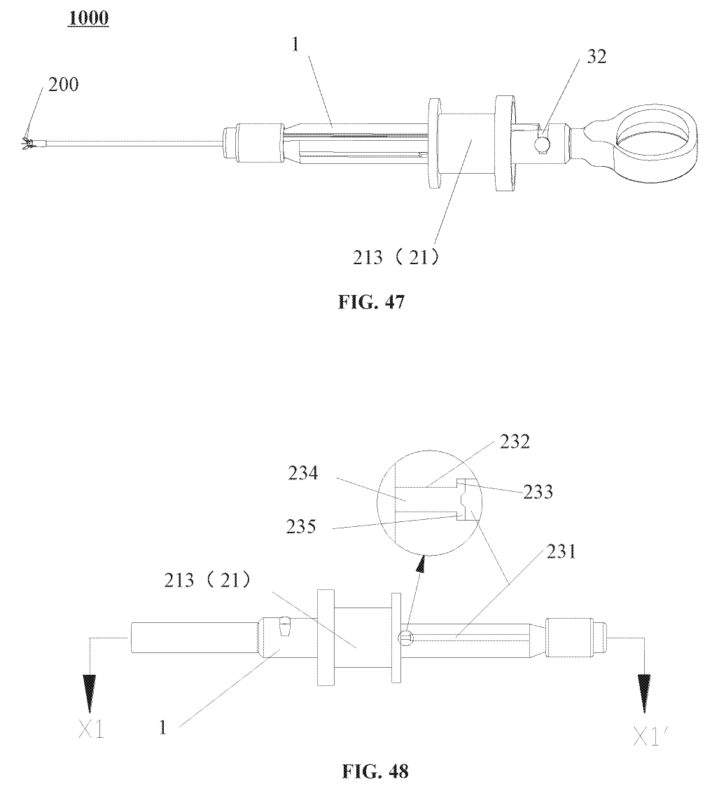

Some embodiments of the present application further provide another method for operating the medical device 1000. In some embodiments, referring to FIG. 47, the medical device 1000 includes the handle 100 and a plurality of moving components 200 according to any of the aforementioned embodiments. The plurality of moving components 200 is connected in a one-to-one correspondence with a plurality of transmission portions 4 of the handle. The adjustment portion 3 of the handle drives different transmission portions 4 to connect to the driving portion 2 of the handle, to drive different moving components 200 to operate.

Further, each transmission portion 4 includes a transmission rod 40 and a flexible pull wire. The transmission rod 40 is connected to the moving component 200 through the flexible pull wire. Consequently, when the transmission rod 40 moves toward the proximal end of the handle to the release space 17, the flexible pull wire may break, thereby disconnecting the transmission rod 40 from the connected moving component. Of course, in other embodiments, the flexible pull wire may also be made of a frangible connection block.

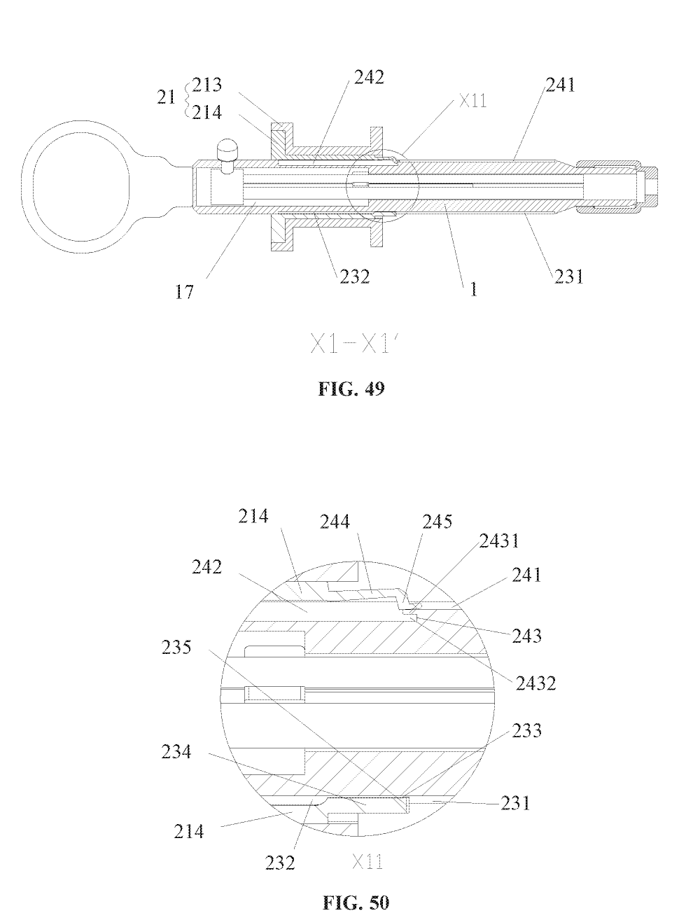

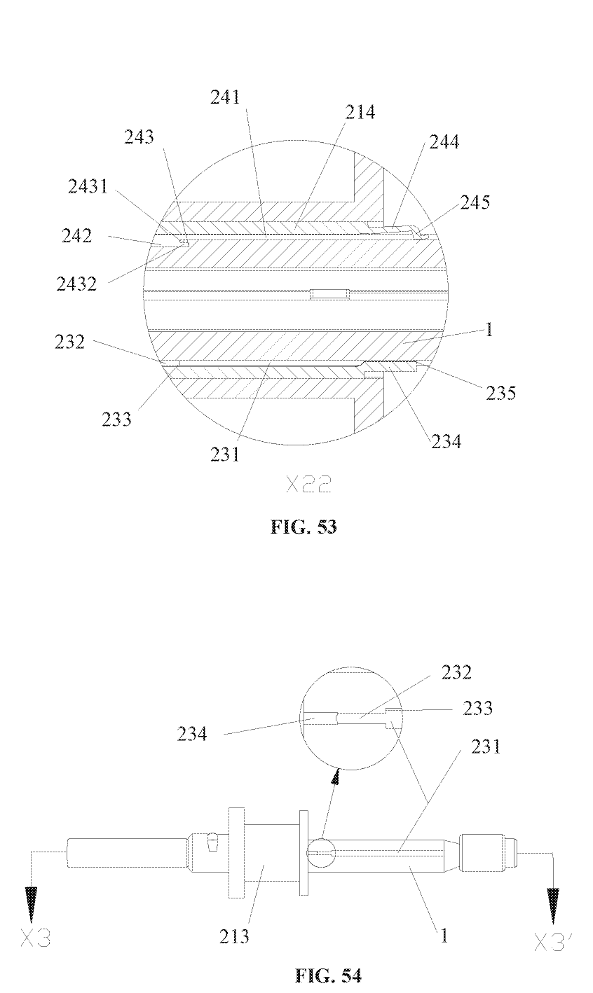

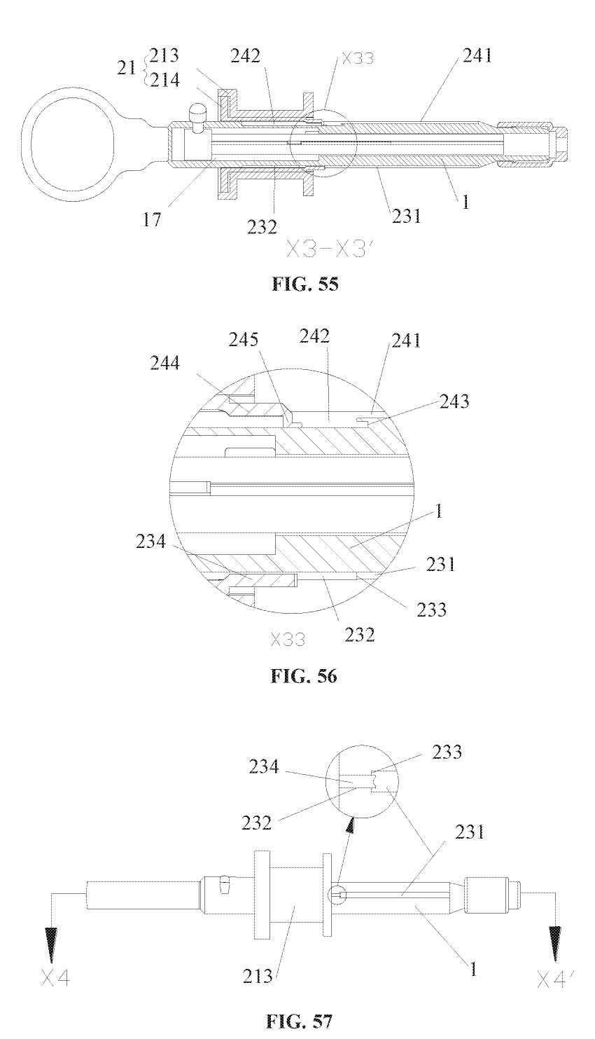

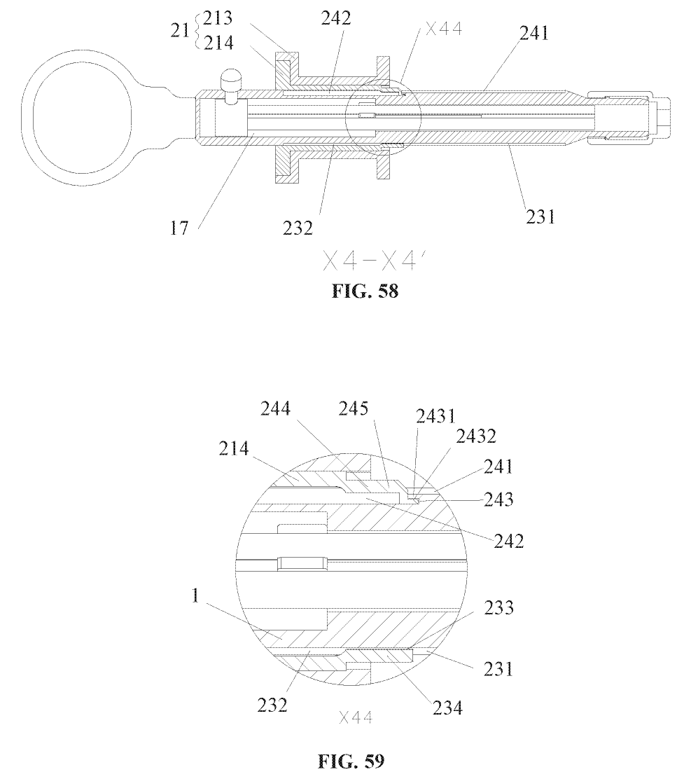

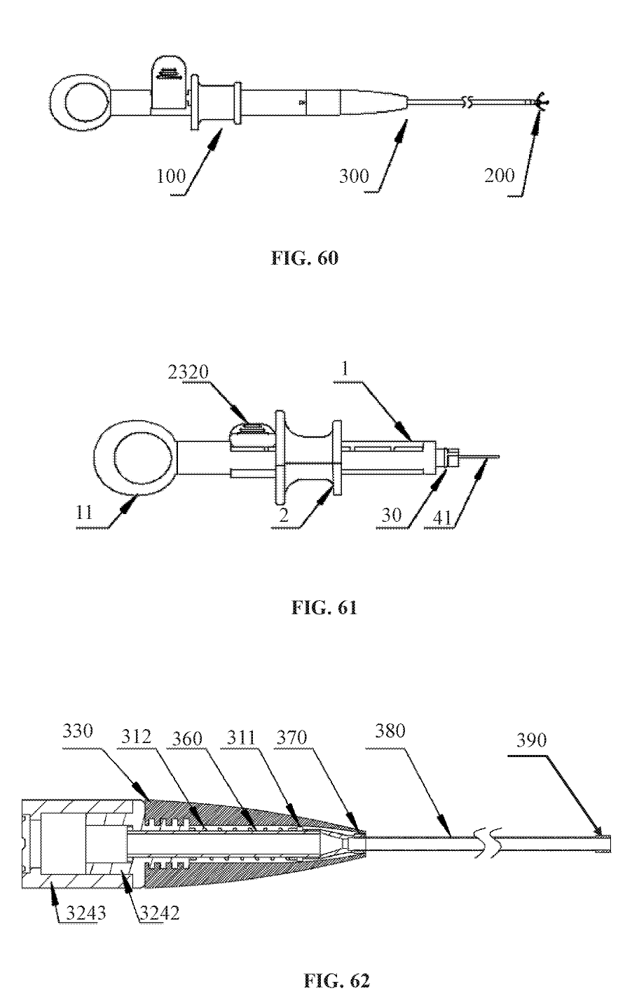

In some embodiments, referring to FIGS. 48-59, the method for operating the medical device 1000 includes:

The driving portion 2 and the transmission portions 4 of the handle are controlled to be in a starting state at an initial position, where the initial position is the preset position.

At the initial position, the adjustment portion 3 is driven to drive different transmission portions 4 to connect to the driving portion 2.

The driving portion 2 drives the connected transmission portions 4 to slide, thereby driving the moving components 200 connected to the transmission portions 4 to operate.

The method may further include that the driving portion 2 is used to drive different connected transmission portions 4 to slide to the release space 17 located on the handheld portion 1, to cause the transmission portions 4 to disengage from the moving components 200 connected thereto.

In some embodiments, when the driving portion 2 and the transmission portions 4 are used to drive the moving components 200 to move, the operation specifically includes:

Sub-step 1.1, causing the driving portion 2 and the transmission portions 4 of the handle to be in the starting state at the initial position. At this time, the first limiting block 235 abuts against the first limiting surface 233, the protrusion 41 aligns with the groove 221, and the second limiting block 245 is located in the third sliding groove 241, as shown in FIGS. 48-50.

Sub-step 1.2, rotating the adjustment portion 3 so that one of the protrusions 41 snaps into the groove 221.

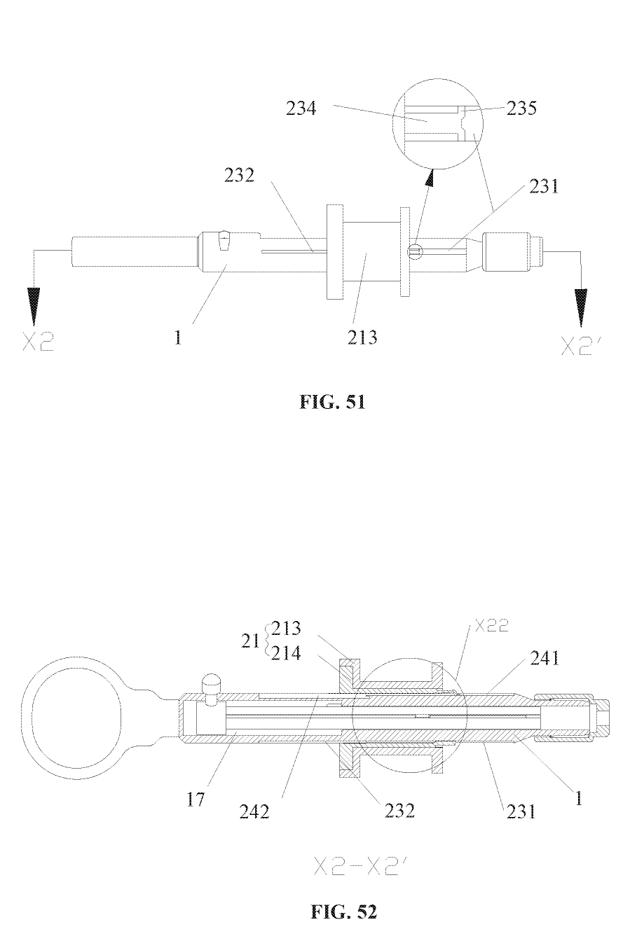

Sub-step 1.3, driving the driving portion 2 so that the driving portion 2 drives the transmission portions 4 to slide synchronously, to drive the moving components 200 connected to the transmission portion 4 to move (e.g., open/close or slide). During this process, the first limiting block 235 moves along the first sliding groove 231, and the second limiting block 245 moves along the third sliding groove 241, as shown in FIGS. 51-53.

Sub-step 1.4, after a movement of the moving components 200 is completed, driving the driving portion 2 toward the proximal end of the handle, so that the driving portion 2 drives the transmission portions 4 to slide synchronously until the first limiting block 235 abuts against the first limiting surface 233 and stops after receiving resistance. At this time, the driving portion 2 and the transmission portions 4 of the handle are in the starting state at the initial position.

Sub-step 1.5, repeating the above sub-steps 1.2 to 1.4.

Further, when the driving portion 2 is used to drive the transmission portions 4 to slide to the release space 17 to cause the transmission portions 4 to disengage from the connected moving components 200, the operation specifically includes:

Sub-step 2.1, causing the driving portion 2 and the transmission portions 4 of the handle to be in the starting state at the initial position. At this time, the first limiting block 235 abuts against the first limiting surface 233, the protrusion 41 aligns with the groove 221, and the second limiting block 245 is located in the third sliding groove 241, as shown in FIGS. 48-50.

Sub-step 2.2, rotating the adjustment portion 3 so that one of the protrusions 41 snaps into the groove 221.

Sub-step 2.3, driving the driving portion 2 toward the proximal end of the handle until the first limiting block 235 is pulled off.

Sub-step 2.4, continuing to drive the driving portion 2 and the transmission portions 4 to move toward the proximal end to the release space 17, and continuing to move toward the proximal end until the transmission portions 4 disengages from the moving components 200 connected thereto. During this process, the first positioning rod 234 moves along the second sliding groove 232, and the second limiting block 245 falls into the fourth sliding groove 242 and moves along the fourth sliding groove 242, as shown in FIGS. 54-56.

Sub-step 2.5, driving the driving portion 2 toward the distal end of the handle, so that the driving portion 2 drives the transmission portions 4 to slide synchronously until the second limiting block 245 abuts against the second limiting surface 243 and stops after receiving resistance. At this time, the driving portion 2 and the transmission portions 4 of the handle are in the starting state at the initial position, as shown in FIGS. 57-59.

Sub-step 2.6, repeating the above sub-steps 2.2, 2.4, and 2.5.

There are various ways for the driving portion 2 and the transmission portions 4 of the handle to be in the starting state at the initial position, besides using the first sliding groove 231, the second sliding groove 232, the first limiting surface 233, the first positioning rod 234, the first limiting block 235, the third sliding groove 241, the fourth sliding groove 242, the second limiting surface 243, the second positioning rod 244, and the second limiting block 245 as in this embodiment. In other embodiments, the elastic reset member 6 may also be used. The operation steps specifically include releasing the driving portion 2, so that the driving portion 2 and the engaged transmission portions 4 return to the initial position under an action of the elastic reset member 6. In other embodiments, the locking member 2320 may also be used to cause the driving portion 2 and the transmission portions 4 of the handle to return to the initial position. Specific steps include moving the driving portion 2 toward the distal end to a distal end of the preset connection position of a locking groove 101, connecting the locking member 2320 to the preset connection position, and then moving the driving portion 2 to abut against the locking member 2320. In other embodiments, the blocking portion 2310 may be used to cause the driving portion 2 and the transmission portions 4 of the handle to return to the initial position. Specific steps include moving the driving portion 2 toward the distal end to a distal end of the blocking portion 2310, rotating the blocking portion 2310 to place the blocking portion 2310 in a blocking position, and then moving the driving portion 2 to abut against the blocking portion 2310. In other embodiments, the limiting step 239 and the elastic piece 236 may also be used to return the driving portion 2 and the transmission portions 4 of the handle to an initial position. Specific steps include moving the driving portion 2 toward the distal end until the blocking portion 2310 of the elastic piece 236 passes over the limiting step 239 and falls into the first sliding segment 237, and then abutting the blocking portion 2310 against the limiting step 239. In some embodiments, the driving portion 2 drives the transmission portions 4 to displace toward the proximal end until a pulling resistance is received, and then reaches the initial position.

It should be noted that different embodiments may produce different beneficial effects. In different embodiments, the beneficial effects may be any one or a combination of the above, or any other possible beneficial effects that may be achieved.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or collocation of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit”, “module”, or “system”. Furthermore, aspects of the present disclosure may take the form of a computer program product embodied in one or more computer readable media having computer-readable program code embodied thereon.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier "about", "approximately", or "substantially" in some examples. Unless otherwise stated, "about", "approximately", or "substantially" indicates that the number is allowed to vary by ±20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. A handle for a medical device, comprising: a handheld portion, a driving portion, and a plurality of transmission portions configured to be operatively coupled to a plurality of moving components at a distal end of the medical device; wherein
the handheld portion is slidably engaged with the driving portion; and
the driving portion is switchably connected to at least one of the plurality of transmission portions, and the driving portion is switchable between being connected to only one of the plurality of transmission portions and being simultaneously connected to at least two of the plurality of transmission portions.

2. The handle according to claim 1, wherein the plurality of transmission portions includes a target transmission portion, and the target transmission portion is engaged with the driving portion;
when the driving portion is driven toward a distal end or a proximal end of the driving portion, the driving portion drives the target transmission portion to slide synchronously along a first direction, so as to drive at least one of the plurality of moving components connected to the target transmission portion of the plurality of moving components to perform an action.

3. The handle according to claim 1, further comprising an adjustment portion, wherein the adjustment portion cooperates with the plurality of transmission portions to drive at least one of the plurality of transmission portions to be switchably connected to the driving portion.

4. The handle according to claim 3, wherein the adjustment portion is rotatably connected to the handheld portion, a rotation axis of the rotatable connection is parallel to a first direction, and the first direction is a sliding direction of a slidable engagement of the handheld portion and the driving portion; and the adjustment portion is slidably engaged with the plurality of transmission portions along the first direction.

5. The handle according to claim 4, wherein at least a portion of the plurality of transmission portions includes a first connection member, the driving portion includes one or more second connection members, and when the adjustment portion rotates relative to the handheld portion about the rotation axis, at least one of a plurality of first connection members is switchably engageable with a corresponding one of the one or more second connection members, such that at least one of the plurality of transmission portions is switchably connected to the driving portion.

6. The handle according to claim 5, wherein when both the driving portion and the first connection member are located at a preset position along the first direction, the adjustment portion is operable to switch the plurality of transmission portions connected to the driving portion.

7. The handle according to claim 6, wherein when the driving portion drives the transmission portion to move from a distal end of the driving portion to the preset position, the transmission portion controls at least one of the plurality of moving components to perform a first action; and/or,
when the driving portion drives the transmission portion to move past the preset position toward a proximal end of the driving portion, the transmission portion controls the at least one of the plurality of moving components to perform a second action.

8. The handle according to claim 6, wherein the handheld portion includes a positioning portion, the positioning portion is disposed on a distal end of the first connection member, and when the distal end of the first connection member abuts against the positioning portion, the first connection member is located at the preset position and is disengaged from the driving portion; and/or,
wherein the handheld portion includes a positioning portion, the positioning portion is disposed on a proximal end of the first connection member, and when the proximal end of the first connection member abuts against the positioning portion, the first connection member is located at the preset position and is disengaged from the driving portion.

9. The handle according to claim 6, further comprising a first positioning assembly, wherein when the driving portion drives the plurality of transmission portions to move from the distal end to the preset position, the first positioning assembly provides a first resistance hindering the driving portion from continuing to move toward the proximal end.

10. The handle according to claim 4, wherein the adjustment portion includes a rotation driving assembly and a support rod, the rotation driving assembly is configured to drive the support rod to rotate when subjected to an external force, the support rod is provided with a plurality of sliding grooves along the first direction, and each of the plurality of transmission portions is slidably engaged with the adjustment portion through one of the plurality of sliding grooves.

11. The handle according to claim 5, wherein the one or more second connection members include a guide groove extending along a circumferential direction of the handheld portion, and after at least one of the plurality of first connection members enters the guide groove, the driving portion is engaged with a corresponding transmission portion.

12. The handle according to claim 11, wherein the guide groove includes a first groove portion and a second groove portion, and the first groove portion and the second groove portion are in communication with each other;
the plurality of transmission portions include a first transmission portion and a second transmission portion, the first connection member corresponding to the first transmission portion includes a first connector, the first connection member corresponding to the second transmission portion includes a second connector, the first connector and the second connector are spaced apart along a circumferential direction of the handheld portion.

13. The handle according to claim 12, wherein the handheld portion further includes at least one axial limiting space, and the at least one axial limiting space is in communication with at least one of the first groove portion and the second groove portion.

14. The handle according to claim 13, wherein a connection state between the plurality of transmission portions and the driving portion includes at least one of a first connection state, a second connection state, or a third connection state,
the first connection state is that the first connector is connected to the driving portion through the first groove portion, and the second connector is connected to the driving portion through the second groove portion;
the second connection state is that the first connector is connected to the driving portion through the second groove portion, and the second connector is located in the at least one axial limiting space; and
the third connection state is that the second connector is connected to the driving portion through the first groove portion, and the first connector is located in the at least one axial limiting space.

15. The handle according to claim 12, wherein at least one of the first connector and the second connector includes a slip ring and a connecting post, the slip ring is sleeved outside the connecting post and is rotatable relative to the connecting post.

16. The handle according to claim 10, wherein the support rod includes a rod body and a support protrusion, and the support protrusion is disposed on an outer surface of the rod body.

17. A medical device, comprising:
a handle, the handle including: a handheld portion, a driving portion, and a plurality of transmission portions; wherein
the handheld portion is slidably engaged with the driving portion; and
the driving portion is switchable between being connected to only one of the plurality of transmission portions and being simultaneously connected to at least two of the plurality of transmission portions;
a delivery assembly, wherein a proximal end of the delivery assembly is connected to the handle; and
a plurality of moving components disposed at a distal end of the delivery assembly, wherein each of the plurality of moving components is drivingly connected to at least one of the plurality of transmission portions.

18. The medical device according to claim 17, wherein the handle includes an adjustment portion, the adjustment portion includes a rotary cap and a support rod connected to each other, and the rotary cap is rotatably connected to a distal end of the handheld portion.

19. The medical device according to claim 17, further comprising a clamp portion, wherein the plurality of moving components include a plurality of clamp jaws of the clamp portion.

20. A method for operating a medical device, the medical device including:
a handle, the handle including a driving portion and a plurality of transmission portions; wherein the driving portion is switchable between being connected to only one of the plurality of transmission portions and being simultaneously connected to at least two of the plurality of transmission portions; and
a plurality of moving components, wherein each of the plurality of moving components is drivingly connected to at least one of the plurality of transmission portions;
the method comprising:
controlling the driving portion and the plurality of transmission portions of the handle to be located at a preset position;
driving the plurality of transmission portions to rotate about a first direction until a target transmission portion of the plurality of transmission portions engages with the driving portion; and
controlling the driving portion to drive the target transmission portion to slide along the first direction to control at least one of the plurality of moving components connected to the target transmission portion to perform an operation.
